(19) United States
(12) Patent Application Publication
(10) Pub. No.: US 2023/0092787 A1
(43) Pub. Date: Mar. 23, 2023

(54) CAR T CELLS TARGETING THE INTEGRIN ALPHAV BETA3 EXHIBIT ROBUST ANTI-TUMOR RESPONSES AGAINST GLIOMAS AND OTHER SOLID TUMOR MALIGNANCIES

(71) Applicant: University of Virginia Patent Foundation, Charlottesville, VA (US)

(72) Inventors: Daniel Warnell Lee, III, Crozet (US); Dustin A. Cobb, Charlottesville (US)

(73) Assignee: University of Virginia Patent Foundation, Charlottesville, VA (US)

(21) Appl. No.: 17/800,387

(22) PCT Filed: Feb. 17, 2021
(86) PCT No.: PCT/US21/18406

§ 371 Date: Aug. 17, 2022

Related U.S. Application Data
(60) Provisional application No. 62/977,573, filed on Feb. 17, 2020.

(51) Int. Cl. A61K 35/17; A61P 35/00; C07K 14/725; C07K 16/28; C12N 15/86

(52) U.S. Cl. CPC A61K 35/17; A61P 35/00; C07K 14/7051; C07K 16/2842; C12N 15/86

(57) ABSTRACT

Methods and compositions for treating cancer, including brain cancers such as diffuse intrinsic pontine glioma (DIPG) and glioblastoma (GBM), breast cancers, melanomas, cervical cancers, bladder cancers, lung cancers, neuroblastomas, and rhabdomyosarcomas (RMS), are described. Also described are methods of preparing cells comprising chimeric antigen receptors (CARs), such as CAR T cells, that target integrin alphav beta3 (αvβ3).

### CROSS REFERENCE TO RELATED APPLICATIONS

This application claims benefit of U.S. Provisional Application Ser. No. 62/977,573 filed Feb. 17, 2020, the disclosure of which is incorporated herein by reference in its entirety.

### REFERENCE TO SEQUENCE LISTING SUBMITTED ELECTRONICALLY

The content of the electronically submitted sequence listing in ASCII text file (Name: 3062-111-PCT.ST25.txt; Size: 12 kilobytes; and Date of Creation: Feb. 17, 2021) filed with the application is incorporated herein by reference in its entirety.

### TECHNICAL FIELD

The presently disclosed subject matter relates to methods of producing chimeric antigen receptor (CAR) immune cells, such as T cells, directed against integrin complex alphaᵥ beta₃ (αᵥβ₃), as well as to methods of treating cancers, including diffuse intrinsic pontine glioma (DIPG) and glioblastoma, using the CAR immune cells, such as T cells.

### Abbreviations

%=percentage
αᵥβ₃=integrin alphaᵥ beta₃ or alphaᵥ beta3
CAR=chimeric antigen receptor
DIPG=diffuse intrinsic pontine glioma
EGF=epidermal growth factor
ELISA=enzyme-linked immunosorbent assay
E:T=effector to target (ratio)
FGF=fibroblast growth factor
GBM=glioblastoma or glioblastoma multiforme
GFP=green fluorescent protein
GM-CSF=granulocyte macrophage-colony stimulating factor
IFN-γ=interferon gamma
IL-2=interleukin-2
IP=intraperitoneal
IT=intratumoral
IU=international units
IVIS=In Vivo Imaging System
kg=kilogram
MFI=mean fluorescence intensity
ml=milliliter
NK=natural killer
NSG=NOD scid gamma
PBMC=peripheral blood mononuclear cell
PDGF=platelet-derived growth factor
pg=picogram
RMS=rhabdomyosarcoma
scFv=single chain variable fragment
THF-1=T cell factor 1
TNF-α=Tumor necrosis factor alpha

### BACKGROUND

Diffuse intrinsic pontine glioma (DIPG) is a highly aggressive tumor that develops in the pons region of the brainstem during early childhood, most commonly in children five to ten years old and accounts for approximately 10 to 20% of childhood brain tumors. Due to is anatomical location, DIPG is inoperable and is only treatable via chemotherapy and radiation. There is currently no cure for DIPG. The median survival rate for DIPG patients is about 9 months following diagnosis and the five-year survival rate is less than 1%. Similarly, glioblastoma (GBM), which is the most common primary brain tumor in adults, is also characterized by very poor survival. There are about 2-3 new cases of GBM per 100,000 adults per year world-wide. The median survival rate for GBM is about 14-15 months following diagnosis.

Accordingly, there is an ongoing need for additional methods of treating these tumors, as well as other cancers.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides an isolated nucleic acid encoding a chimeric antigen receptor (CAR), wherein the CAR comprises an integrin alphaᵥ beta₃ (αᵥβ₃) binding domain, a transmembrane domain, a cytoplasmic co-stimulatory (CS) domain, and an intracellular signaling domain, wherein the CAR is free of an extracellular immunoglobulin G4 (IgG4) hinge domain and/or wherein the integrin αᵥβ₃ binding domain is a single chain variable fragment (scFV) comprising a heavy chain variable region (V_{H}) encoded by SEQ ID NO: 3 or by a sequence at least 90% identical to SEQ ID NO: 3 and a light chain variable region (V_{L}) encoded by SEQ ID NO: 5 or by a sequence at least 90% identical to SEQ ID NO: 5. In some embodiments, the CAR is free of an IgG4 hinge domain.

In some embodiments, the transmembrane domain comprises a cluster of differentiation 8 alpha (CD8α) transmembrane domain. In some embodiments, the transmembrane domain is a peptide encoded by SEQ ID NO: 7 or by a sequence at least 90% identical to SEQ ID NO: 7.

In some embodiments, the integrin αᵥβ₃ binding domain comprises a scFV comprising a V_{H} encoded by SEQ ID NO: 3 and a V_{L} encoded by SEQ ID NO: 5. In some embodiments, the integrin αᵥβ₃ binding domain comprises a linker peptide located between SEQ ID NO: 3 and SEQ ID NO: 5, optionally wherein said linker peptide is encoded by SEQ ID NO: 4 or by a sequence at least 90% identical to SEQ ID NO: 4. In some embodiments, the integrin αᵥβ₃ binding domain is attached to the transmembrane domain via a peptide encoded by SEQ ID NO: 6 or by a sequence at least 90% identical to SEQ ID NO: 6.

In some embodiments, the cytoplasmic CS domain comprises one or both of a cluster of differentiation 28 (CD28) signaling domain and a tumor necrosis factor superfamily member 9 (4-1BB) signaling domain, optionally wherein the CD28 signaling domain is encoded by SEQ ID NO: 8 or a sequence at least 90% identical to SEQ ID NO: 8 and/or wherein the 4-1 BB signaling domain is encoded by SEQ ID NO: 9 or a sequence at least 90% identical to SEQ ID NO: 9. In some embodiments, the intracellular signaling domain is a cluster of differentiation 247 (CD3ζ) activation domain, optionally wherein the intracellular signaling domain is the peptide encoded by SEQ ID NO: 10 or a sequence at least 90% identical to SEQ ID NO: 10. In some embodiments, the isolated nucleic acid further comprises a signal peptide, optionally wherein the signal peptide is a granulocyte-macrophage colony-stimulating factor (GM-CSF) signal peptide, further optionally wherein the signal peptide is human GM-CSF signal peptide encoded by SEQ ID NO: 2.

In some embodiments, the isolated nucleic acid comprises, in the following 5′ to 3′ order: (i) SEQ ID NO: 2; (ii) SEQ ID NO: 3; (iii) SEQ ID NO: 4; (iv) SEQ ID NO: 5; (v) SEQ ID NO: 6; (vi) SEQ ID NO: 7; (vii) SEQ ID NO: 8 and/or SEQ ID NO: 9; and (vii) SEQ ID NO: 10.

In some embodiments, the presently disclosed subject matter provides a nucleic acid construct comprising an isolated nucleic acid encoding a CAR, wherein the CAR comprises an integrin αᵥβ₃ binding domain, a transmembrane domain, a cytoplasmic CS domain, and an intracellular signaling domain, wherein the CAR is free of an extracellular IgG4 hinge domain and/or wherein the integrin αᵥβ₃ binding domain is a scFV comprising a V_{H} encoded by SEQ ID NO: 3 or by a sequence at least 90% identical to SEQ ID NO: 3 and a V_{L} encoded by SEQ ID NO: 5 or by a sequence at least 90% identical to SEQ ID NO: 5, wherein said isolated nucleic acid is operably linked to a promoter.

In some embodiments, the presently disclosed subject matter provides a vector comprising an isolated nucleic acid encoding a CAR, wherein the CAR comprises an integrin αᵥβ₃ binding domain, a transmembrane domain, a cytoplasmic CS domain, and an intracellular signaling domain, wherein the CAR is free of an extracellular IgG4 hinge domain and/or wherein the integrin αᵥβ₃ binding domain is a scFV comprising a V_{H} encoded by SEQ ID NO: 3 or by a sequence at least 90% identical to SEQ ID NO: 3 and a V_{L} encoded by SEQ ID NO: 5 or by a sequence at least 90% identical to SEQ ID NO: 5, optionally wherein the vector is a retroviral vector.

In some embodiments, the presently disclosed subject matter provides a CAR, wherein the CAR comprises an integrin αᵥβ₃ binding domain, a transmembrane domain, at least one cytoplasmic CS domain, and an intracellular signaling domain, wherein the CAR is free of an extracellular IgG4 hinge domain and/or wherein the integrin αᵥβ₃ binding domain is a scFV comprising: (i) a V_{H} comprising SEQ ID NO: 13 or an amino acid sequence at least 90% identical to SEQ ID NO: 13, and a V_{L} comprising SEQ ID NO: 15 or an amino acid sequence at least 90% identical to SEQ ID NO: 15; and/or (ii) a V_{H} comprising complementarity determining regions CDR-VH1, CDR-VH2, and CDR-VH3, wherein CDR-VH1, CDR-VH2, and CDR-VH3 comprise, consist essentially of, or consist of amino acids 31-35, 50-66, and 99-106 of SEQ ID NO: 13, respectively; and/or (iii) a V_{L} comprising complementarity determining regions CDR-VL1, CDR-VL2, and CDR-VL3, wherein CDR-VL1, CDR-VL2, and CDR-VL3 comprise, consist essentially of, or consist of amino acids 24-34, 50-56, and 88-97 of SEQ ID NO: 15, respectively. In some embodiments, the CAR is free of an IgG4 hinge domain.

In some embodiments, the transmembrane domain comprises a CD8α transmembrane domain. In some embodiments, the transmembrane domain comprises SEQ ID NO: 7 or an amino acid sequence at least 90% identical to SEQ ID NO: 17.

In some embodiments, the integrin αᵥβ₃ binding domain comprises a scFV comprising a V_{H} comprising SEQ ID NO: 13 and a V_{L} comprising SEQ ID NO: 15. In some embodiments, the integrin αᵥβ₃ binding domain comprises a linker peptide located between SEQ ID NO: 13 and SEQ ID NO: 15, optionally wherein said linker peptide comprises or consists of SEQ ID NO: 14 or an amino acid sequence at least 90% identical to SEQ ID NO: 14. In some embodiments, the integrin αᵥβ₃ binding domain is attached to the transmembrane domain via a peptide comprising SEQ ID NO: 16 or by an amino acid sequence at least 90% identical to SEQ ID NO: 16.

In some embodiments, the cytoplasmic CS domain comprises one or both of a CD28 signaling domain and a 4-1BB signaling domain, optionally wherein the CD28 signaling domain comprises SEQ ID NO: 18 or an amino acid sequence at least 90% identical to SEQ ID NO: 18 and/or wherein the 4-1 BB signaling domain comprises SEQ ID NO: 19 of an amino acid sequence at least 90% identical to SEQ ID NO: 19. In some embodiments, the intracellular signaling domain is a CD3ζ activation domain, optionally wherein the intracellular signaling domain comprises SEQ ID NO: 20 or an amino acid sequence at least 90% identical to SEQ ID NO: 20. In some embodiments, the CAR further comprises a signal peptide, optionally wherein the signal peptide is a GM-CSF signal peptide, further optionally wherein the signal peptide comprises SEQ ID NO: 12.

In some embodiments, the CAR comprises, in the following N-terminus to C-terminus order: (i) SEQ ID NO: 12; (ii) SEQ ID NO: 13; (iii) SEQ ID NO: 14; (iv) SEQ ID NO: 15; (v) SEQ ID NO: 16; (vi) SEQ ID NO: 17; (vii) SEQ ID NO: 18 and/or SEQ ID NO: 19; and (vii) SEQ ID NO: 20.

In some embodiments, the presently disclosed subject matter provides an isolated polynucleotide encoding a CAR comprising an integrin αᵥβ₃ binding domain, a transmembrane domain, at least one cytoplasmic CS domain, and an intracellular signaling domain, wherein the CAR is free of an extracellular IgG4 hinge domain and/or wherein the integrin αᵥβ₃ binding domain is a scFV comprising a V_{H} comprising SEQ ID NO: 13 or an amino acid sequence at least 90% identical to SEQ ID NO: 13, and a V_{L} comprising SEQ ID NO: 15 or an amino acid sequence at least 90% identical to SEQ ID NO: 15.

In some embodiments, the presently disclosed subject matter provides a genetically modified immune cell comprising an isolated nucleic acid encoding a CAR, wherein the CAR comprises an integrin αᵥβ₃ binding domain, a transmembrane domain, a cytoplasmic CS domain, and an intracellular signaling domain, wherein the CAR is free of an extracellular IgG4 hinge domain and/or wherein the integrin αᵥβ₃ binding domain is a scFV comprising a V_{H} encoded by SEQ ID NO: 3 or by a sequence at least 90% identical to SEQ ID NO: 3 and a V_{L} encoded by SEQ ID NO: 5 or by a sequence at least 90% identical to SEQ ID NO: 5; or a vector thereof. In some embodiments, the genetically modified immune cell expresses the CAR encoded by the isolated nucleic acid. In some embodiments, the presently disclosed subject matter provides a genetically modified immune cell comprising a CAR, wherein the CAR comprises an integrin αᵥβ₃ binding domain, a transmembrane domain, at least one cytoplasmic CS domain, and an intracellular signaling domain, wherein the CAR is free of an extracellular IgG4 hinge domain and/or wherein the integrin αᵥβ₃ binding domain is a scFV comprising a V_{H} comprising SEQ ID NO: 13 or an amino acid sequence at least 90% identical to SEQ ID NO: 13, and a V_{L} comprising SEQ ID NO: 15 or an amino acid sequence at least 90% identical to SEQ ID NO: 15.

In some embodiments, the presently disclosed subject matter provides a population of immune cells comprising a plurality of the presently disclosed genetically modified immune cells. In some embodiments, the presently disclosed subject matter provides a pharmaceutical composition comprising the presently disclosed genetically modified immune cells and a pharmaceutically acceptable excipient.

In some embodiments, the presently disclosed subject matter provides a method of treating cancer in a subject in need thereof, comprising administering a therapeutically effective amount of the presently disclosed genetically modified immune cells. In some embodiments, the cancer is selected from a brain cancer, a breast cancer, a cervical cancer, a bladder cancer, a lung cancer, melanoma, neuroblastoma, and rhabdomyosarcoma (RMS), optionally wherein said lung cancer is a non-small cell lung cancer or wherein said breast cancer is a triple negative breast cancer.

In some embodiments, the cancer is a brain cancer. In some embodiments, the brain cancer is a diffuse intrinsic pontine glioma (DIPG). In some embodiments, the brain cancer is a glioblastoma (GBM).

In some embodiments, the administering is performed intratumorally. In some embodiments, the administering is performed systemically.

In some embodiments, the presently disclosed subject matter provides a method of making an anti-integrin αᵥβ₃ CAR immune cell, wherein the method comprises introducing into an immune cell a nucleic acid construct comprising an isolated nucleic acid encoding a CAR, wherein the CAR comprises an integrin αᵥβ₃ binding domain, a transmembrane domain, a cytoplasmic CS domain, and an intracellular signaling domain, wherein the CAR is free of an extracellular IgG4 hinge domain and/or wherein the integrin αᵥβ₃ binding domain is a scFV comprising a V_{H} encoded by SEQ ID NO: 3 or by a sequence at least 90% identical to SEQ ID NO: 3 and a V_{L} encoded by SEQ ID NO: 5 or by a sequence at least 90% identical to SEQ ID NO: 5, wherein said isolated nucleic acid is operably linked to a promoter, or a vector thereof. In some embodiments, the nucleic acid construct or vector is introduced into the immune cell ex vivo. In some embodiments, the vector is a virus vector, optionally wherein the virus vector is a retrovirus vector. In some embodiments, the immune cell is a T cell.

In some embodiments, the presently disclosed subject matter provides a method of treating cancer in a subject in need thereof, wherein the method comprises: (a) obtaining a population of human immune cells; (b) transducing at least a portion of the population of human immune cells with a vector of the presently disclosed subject matter to provide a population of transduced human immune cells; and (c) administering the population of transduced human immune cells to the subject. In some embodiments, the population of human immune cells is a population of human T cells obtained by: (a1) obtaining cells from human blood serum, optionally wherein said cells comprise peripheral blood mononuclear cells (PBMCs); and (a2) treating the cells from (a1) to isolate a population of cells enriched for central memory T cells; memory stem T cells, and naive T cells.

In some embodiments, the population of human immune cells are autologous to the subject. In some embodiments, the population of human immune cells are allogenic to the subject.

In some embodiments, the cancer is selected from a brain cancer, a breast cancer, a cervical cancer, a bladder cancer, a lung cancer, melanoma, neuroblastoma, and RMS, optionally wherein said lung cancer is a non-small cell lung cancer or wherein said breast cancer is a triple negative breast cancer. In some embodiments, the cancer is a brain cancer. In some embodiments, the brain cancer is a DIPG. In some embodiments, the brain cancer is a GBM. In some embodiments, the administering is performed intratumorally. In some embodiments, the administering is performed systemically.

In some embodiments, the presently disclosed subject matter provides a nucleic acid that encodes an antibody that binds to integrin αᵥβ₃, wherein the nucleic acid comprises SEQ ID NO: 3 or a sequence at least 90% identical to SEQ ID NO: 3 and/or SEQ ID NO: 5 or a sequence at least 90% identical to SEQ ID NO: 5. In some embodiments, the antibody is a scFV. In some embodiments, the nucleic acid comprises the sequence R₁-R₂-R₃, wherein: R₁ is SEQ ID NO: 3 or a sequence at least 90% identical thereto; R₂ is SEQ ID NO: 4 or a sequence at least 90% identical thereto; and R₃ is SEQ ID NO: 5 or a sequence at least 90% identical thereto.

In some embodiments, the presently disclosed subject matter provides an antibody that binds to integrin αᵥβ₃, wherein the antibody comprises: (i) a V_{H} comprising SEQ ID NO: 13 or a sequence at least 90% identical to SEQ ID NO: 13 and/or a V_{L} comprising SEQ ID NO: 15 or a sequence at least 90% identical to SEQ ID NO: 15; and/or (ii) a V_{H} comprising complementarity determining regions CDR-VH1, CDR-VH2, and CDR-VH3, wherein CDR-VH1, CDR-VH2, and CDR-VH3 comprise, consist essentially of, or consist of amino acids 31-35, 50-66, and 99-106 of SEQ ID NO: 13, respectively; and/or (iii) a V_{L} comprising complementarity determining regions CDR-VL1, CDR-VL2, and CDR-VL3, wherein CDR-VL1, CDR-VL2, and CDR-VL3 comprise, consist essentially of, or consist of amino acids 24-34, 50-56, and 88-97 of SEQ ID NO: 15, respectively. In some embodiments, the antibody is a scFV. In some embodiments, the antibody comprises a polypeptide sequence R₄-R₅-R₆, wherein: R₄ is SEQ ID NO: 13 or a sequence at least 90% identical thereto; R₅ is SEQ ID NO: 14 or a sequence at least 90% identical thereto; and R₆ is SEQ ID NO: 15 or a sequence at least 90% identical thereto.

Accordingly, it is an object of the presently disclosed subject matter to provide nucleic acids encoding CARs targeting integrin αᵥβ₃, CARs targeting integrin αᵥβ₃, related antibodies, and related methods of treating cancer and generating CAR T cells. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE DRAWINGS

FIG. 1: Tumor cell line expression of integrin αᵥβ₃ measured by flow cytometry. Cell surface expression of integrin αᵥβ₃ evaluated by flow cytometry staining with phycoerythrin-conjugated anti-integrin αᵥβ₃ monoclonal antibody (clone LM609) on diffuse intrinsic pontine glioma (DIPG), neuroblastoma, glioblastoma (GBM), triple-negative breast cancer, bladder cancer, rhabdomyosarcoma (RMS), lung cancer, and cervical squamous cell carcinoma tumor cell lines. Unstained cells (dotted line histograms); isotype control antibody stained cells (gray-shaded histograms); anti-integrin αᵥβ₃ stained cells (dark-shaded histograms).

FIG. 2: Minimal expression of integrin αᵥβ₃ in normal organ tissues. Immunohistochemical staining for integrin αᵥβ₃ expression was conducted on a tissue microarray containing cores obtained from normal healthy donors (five samples per organ type) and, as a positive control, on a sample from a human diffuse intrinsic pontine glioma (DIPG) mouse xenograft tumor. Staining intensity was analyzed using digital histopathology software to obtain a H-score staining index for each sample. Donors are represented in the graph by individual circles.

FIGS. 3A and 3B: Minimal mRNA transcript expression of genes encoding ITGAV and ITGB3 in normal organ tissues. FIG. 3A shows gene expression for ITGAV in normal human tissues and FIG. 3B shows gene expression for ITGB3 in normal human tissues. ITGAV and ITGB3 are the genes encoding the integrin αᵥβ₃ protein.

FIG. 4: Schematic diagram of an integrin αᵥβ₃ chimeric antigen receptor (CAR) of the presently disclosed subject matter.

FIGS. 5A and 5B: Schematic diagrams of integrin αᵥβ₃ chimeric antigen receptor (CAR) constructs of the presently disclosed subject matter for expression in retroviral vectors. FIG. 5A is a schematic of the construction of an anti-integrin αᵥβ₃ CAR T cell construct comprising a CD28 co-stimulatory domain referred to herein as αᵥβ₃.28z and FIG. 5B is a schematic of the construction of an anti-αᵥβ₃.BBz CAR T cell construct comprising a 4-1BB co-stimulatory domain referred to herein as αᵥβ₃.BBz. Identical core sequences can be expressed in lentiviral vectors. Human nucleotide sequences of granulocyte-macrophage colony-stimulating factor (GM-CSF) receptor signal peptide, CD8 alpha transmembrane domain, CD28, 4-1BB, and CD3 zeta genes were obtained from the Consensus Coding Sequence database (National Center for Biology Information (NCBI)). The assembled domains of the CAR constructs were generated by commercial gene synthesis and sub-cloned into a retroviral vector using blunt-ended cloning.

FIGS. 6A and 6B: High-level cell surface expression of integrin αᵥβ₃ chimeric antigen receptor (CAR) in human T cells following retroviral transduction. Peripheral blood mononuclear cells (PBMCs) were stimulated for three days with anti-CD3/anti-CD28-coated magnetic beads in the presence of human interleukin 2 (IL-2) (40 international units per milliliter (IU/ml). Stimulated PBMCs were then transduced via retronectin-bound retrovirus containing integrin αᵥβ₃ CAR constructs on two consecutive days. Following expansion, CAR surface expression was measured by flow cytometry by incubating cells with biotinylated-L protein flowed by staining with fluorochrome-conjugated streptavidin and anti-CD3 antibody. FIG. 6A shows chromatograms from the flow cytometry of cells transduced with retrovirus containing the αᵥβ₃.28z and αᵥβ₃.BBz CAR constructs shown in FIGS. 5A and 5B or a control construct where the integrin αᵥβ₃ binding domain is replaced by a CD19 binding domain (CD19.28z CAR). Results are also shown from non-transduced cells. FIG. 6B is a graph showing the percentage of CAR+ T cells in three normal donors across multiple experiments. Error bars indicate standard deviation.

FIGS. 7A-7C: Robust expansion of integrin αᵥβ₃ chimeric antigen receptor (CAR) T cells following retroviral transduction. FIG. 7A shows T cell expansion quantified at multiple time points following retroviral transduction of T cells from three individual normal donors with vectors comprising the αᵥβ₃.28z CAR and αᵥβ₃.BBz CAR constructs shown in FIGS. 5A and 5B or a CD19 CAR construct (CD19.28z CAR). Data is also shown for non-transduced cells. Data from T cells from each individual donor is shown in a separate graph (top, middle, and bottom). FIG. 7B is a pair of graphs showing the frequencies (percentage of cells) of CD8⁺ (top) or CD4⁺ (bottom) memory T cell subsets following CAR T cell expansion protocol. Data is shown following retroviral transduction of T cells from three normal donors with vectors comprising the αᵥβ₃.28z CAR and αᵥβ₃.BBz CAR constructs shown in FIGS. 5A and 5B. FIG. 7C is a pair of graphs of the mean fluorescence intensity (MFI) of programmed cell death protein 1 (PD-1), lymphocyte-activation gene 3 (LAG-3), and T-cell immunoglobulin and mucin-domain containing-3 (TIM-3) on CD4⁺ (top) or CD8⁺ (bottom) CAR T cells following expansion protocol. Data is shown following retroviral transduction of T cells from three normal donors with vectors comprising the αᵥβ₃.28z CAR and αᵥβ₃.BBz CAR constructs shown in FIGS. 5A and 5B or a CD19 CAR construct (CD19.28z CAR). Data is also shown for non-transduced cells.

FIGS. 8A-8D: In vitro cytotoxicity of integrin αᵥβ₃ chimeric antigen receptor (CAR) T cells against diffuse intrinsic pontine glioma (DIPG) tumor cell lines. αᵥβ₃.28z CAR T cells, αᵥβ₃.BBz CAR T cells, control CD19.28z CAR T cells, or non-transduced T cells were co-cultured overnight with four different DIPG tumor cell lines (SU-DIPG-IV, FIG. 8A; SU-DIPG-XIII, FIG. 8B; SU-DIPG-36, FIG. 8C; and SU-DIPG-XVII, FIG. 8D) expressing firefly luciferase at various effector to target (E:T) cell ratios (10:1; 5:1; and 2.5:1). Anti-tumor cytotoxicity was evaluated by measuring bioluminescent activity of the remaining tumor cells following the addition of luciferin. Cytotoxicity was calculated as follows: percent cytotoxicity=100−(((average signal of CAR T cell-treated wells)/(average signal from untreated tumor cell only wells))×100). Each graph is representative of two or three individual experiments performed with CAR T cells generated from multiple donors.

FIGS. 9A-9C: High-levels of effector cytokine production by integrin αᵥβ₃ chimeric antigen receptor (CAR) T cells following in vitro exposure to diffuse intrinsic pontine glioma (DIPG) cell lines. αᵥβ₃.28z CAR T cells, αᵥβ₃.BBz CAR T cells, control CD19.28z CAR T cells or non-transduced T cells were co-cultured for 24 hours with different DIPG tumor cell lines (SU-DIPG-IV, SU-DIPG-XIII, SU-DIPG-36 and SU-DIPG-17) at a 1:1 effector to target cell ratio. Culture supernatants were harvested and analyzed for effector cytokines interferon-gamma (IFN-γ; FIG. 9A), interleukin-2 (IL-2; FIG. 9B), and tumor necrosis factor-alpha (TNF-α; FIG. 9C) by ELISA. Cytokines levels are presented as picograms per milliliter (pg/ml).

FIGS. 10A and 10B: Treatment with integrin αᵥβ₃ chimeric antigen receptor (CAR) T cells mediates tumor regression in mice engrafted with human diffuse intrinsic pontine glioma (DIPG) tumors. NOD scid gamma (NSG) immunodeficient mice were implanted with 0.5×10⁶ Su-DIPG-36 cells expressing green fluorescent protein (GFP)/firefly luciferase into the pons via stereotactic injection. Three weeks later, 2×10⁶ αᵥβ₃.28z CAR T cells, αᵥβ₃.BBz CAR T cells, or control CD19.28z CAR T cells were injected intratumorally via stereotactic injection. Tumor burden in mice was monitored longitudinally via bioluminescent imaging. FIG. 10A shows a series of representative images of mouse bioluminescent imaging on Day 0 (pre-treatment), 7, 14, 21, 35, and 56 post treatment. Color scales are shown to the right of each group indicating the minimum and maximum radiance values detected. FIG. 10B is a graph showing tumor burden (radiance) over time. Tumor regression is evident in NSG mice treated with αᵥβ₃.28z CAR T cells and αᵥβ₃.BBz CAR T cells by Day 2 post-treatment and continues to decrease for up to 2 weeks. Data represent the means for each treatment group (αᵥβ₃.28z CAR T cells (squares); αᵥβ₃.BBz CAR T cells (triangles); and CD10.28z CAR T cells (circles)). Error bars are standard error of the mean (SEM). *p<0.05, **p<0.01, ****p<0.0001 determined by Two-ANOVA with Tukey's multiple comparisons test.

FIGS. 11A-11D: Increased survival of mice engrafted with human diffuse intrinsic pontine glioma (DIPG) tumors following treatment with integrin αᵥβ₃ chimeric antigen receptor (CAR) T cells, but not control CD19 CAR T cells. Survival curves of mice treated intratumorally with 2×10⁶ αᵥβ₃.28z CAR T cells (squares), αᵥβ₃.BBz CAR T cells (triangles) or control CD19.28z CAR T cells (circles) three weeks after tumor cell implantation. FIG. 11A shows progression-free survival analysis of mice bearing DIPG tumors following CAR T cell treatment. FIG. 11B shows overall survival analysis of mice following CAR T cell treatment. Results from three independent experiments were pooled (CD19.28z, n=11; αᵥβ₃.28z, n=13; αᵥβ₃.BBz, n=14) and Kaplan-Meier survival analysis was performed with Log-rank (Mantel-Cox) test for comparison between treatment groups. FIG. 11C shows a graph of circulating human T cells (CD45+CD3+) analyzed by flow cytometry from peripheral blood was collected on day 7 and day 14 post-treatment with αᵥβ₃.28z CAR T cells (squares), αᵥβ₃.BBz CAR T cells (triangles) or control CD19.28z CAR T cells. Individual symbols in graph represent individual mice. FIG. 11D is a pair of graphs showing the mean fluorescence intensity (MFI) of programmed cell death protein 1 (PD-1) on CD8⁺ and CD4⁺ CAR T cells (stained with protein L) from peripheral blood of treated mice analyzed by flow cytometry on day 7 and day 14 post-treatment with αᵥβ₃.28z CAR T cells (open bars), or αᵥβ₃.BBz CAR T cells (shaded bars).

FIGS. 12A and 12B. Potent in vitro anti-tumor activity of integrin αᵥβ₃ chimeric antigen receptor (CAR) T cells against glioblastoma (GBM) tumor cell line U87 and U251. FIG. 12A is a pair of graphs showing antitumor activity of tumor cells treated with different CAR T cells. αᵥβ₃.28z CAR T cells, αᵥβ₃.BBz CAR T cells, control CD19.28z CAR T cells or non-transduced T cells were co-cultured overnight with GBM U87 (left) or GBM Y251 (right) tumor cells expressing firefly luciferase, at various effector to target (E:T) cell ratios. Anti-tumor cytotoxicity was then evaluated by measuring bioluminescent activity of remaining tumor cells following addition of luciferin. Culture plates were subsequently read and cytotoxicity was calculated as follows: percent cytotoxicity=100−(((average signal of CAR T cell-treated wells)/(average signal from untreated tumor cell only wells))×100). FIG. 12B is a series of graphs showing the levels (in picograms per milliliter (pg/ml)) of inflammatory cytokines (interferon-gamma (IFN-γ, upper left); interleukin-2 (IL-2, upper right); and tumor necrosis factor-alpha (TNF-α, bottom) in cell supernatants from GBM tumor cells from GBM tumor cell line U87 co-cultured for 24 hours with αᵥβ₃.28z CAR T cells, αᵥβ₃.BBz CAR T cells, control CD19.28z CAR T cells or non-transduced T cells at a 1:1 effector to target cell ratio. αᵥβ₃ CARs produced significantly more inflammatory cytokines than controls with exposed to tumor cells.

FIGS. 13A and 13B: In vivo elimination of tumors by integrin αᵥβ₃ chimeric antigen receptor (CAR) T cells in glioblastoma (GBM)-bearing mice. FIG. 13A is a series of images of NOD scid gamma (NSG) immunodeficient mice with 0.5×10⁶ GBM U87 expressing green fluorescent protein (GFP)/firefly luciferase implanted into the forebrains via stereotactic injection and then, three weeks later, injected intratumorally via stereotactic injection with 2×10⁶ αᵥβ₃.28z CAR T cells, αᵥβ₃.BBz CAR T cells, or control CD19.28z CAR T cells. Tumor burden in mice was measured longitudinally via bioluminescent imaging. FIG. 13B is a graph showing the average tumor burden (radiance) in the mice described for FIG. 13A over time. Error bars represent standard error of the mean (SEM). Data are representative of two independent experiments. *p<0.05, ****p<0.0001 determined by Two-ANOVA with Tukey's multiple comparisons test.

FIG. 14: Increased survival of mice engrafted with human glioblastoma (GBM) tumors following treatment with integrin (1,133 chimeric antigen receptor (CAR) T cells, but not control CD19 CAR T cells. Survival curves of mice treated intratumorally with 2×10⁶ αᵥβ₃ 28z CAR T cells, αᵥβ₃.BBz CAR T cells, or control CD19.28z CAR T cells three weeks after tumor cell implantation. Kaplan-Meier survival analysis was performed with Log-rank (Mantel-Cox) test for comparison between treatment groups.

FIGS. 15A-15E: In vivo persistence and memory phenotype of integrin αᵥβ₃ chimeric antigen receptor (CAR) T cells following clearance of glioblastoma (GBM) tumor cells in xenografted NOD scid gamma (NSG) mice. On day 42 post-treatment with CAR T cells via intratumoral injection, blood was collected from NSG mice and analyzed by flow cytometry for the presence of human CAR T cells. Cells were stained with biotinylated-L protein followed by staining with fluorochrome-conjugated streptavidin, anti-CD45, anti-CD3, and anti-CD127 antibodies. FIG. 15A shows the percentage of human T cells, identified by CD45 and CD3 in the blood of each mouse. FIG. 15B shows the percentage of CAR T cells expressing the memory marker CD127 (IL-7Rα) in each mouse. Error bars indicate standard error of the mean (SEM). Data are representative of two independent experiments. FIG. 15C is a graph of T-cell factor 1 (TCF-1) mean fluorescence intensity (MFI) was calculated in CD127⁺PD-1⁺ and CD127⁻PD-1⁺ populations of CD8⁺ CAR T cells for individual mice treated with αᵥβ₃.28z CAR T cells (squares), αᵥβ₃.BBz CAR T cells (triangles), or control CD19.28z CAR T cells (circles). p values *p<0.05, **p<0.01 were determined by One-Way ANOVA with Tukey's multiple comparisons test. FIG. 15D is a series of graphs of peripheral blood (top left), spleen (top right), and brain (bottom) were harvested on day 70 post-treatment with αᵥβ₃.28z CAR T cells (squares) or αᵥβ₃.BBz CAR T cells (triangles) and analyzed by flow cytometry for the presence of human T cells. Data are presented as means±SEM and symbols in graphs represent individual mice. FIG. 15E is a series of graphs of memory CAR T cell populations (CCR7⁺CD127⁺, CCR7⁺CD127⁻, CCR7⁻, CD127⁺, and CCR7⁻CD127⁻) analyzed on day 70 post-treatment with αᵥβ₃.28z CAR T cells (squares) or αᵥβ₃.BBz CAR T cells (triangles) in blood (top), spleen (middle), and brain (bottom) by gating on CD45⁺CD3⁺CAR⁺ cells. Data are presented as means±SEM and symbols in graphs represent individual mice.

FIGS. 16A and 16B: In vivo cytotoxicity of systemically administered integrin αᵥβ₃ chimeric antigen receptor (CAR) T cells against glioblastoma (GBM) tumors. FIG. 16A is a series of images of NOD scid gamma (NSG) immunodeficient mice with 0.5×10⁶ GBM U87 cells expressing green fluorescent protein (GFP)/firefly luciferase implanted into the forebrains via stereotactic injection and, three weeks later, with 10×10⁶ αᵥβ₃.BBz CAR T cells or control CD19.28z CAR T cells injected intravenously. Tumor burden in mice was monitored longitudinally via bioluminescent imaging. FIG. 16B is a graph showing the average tumor burden (radiance) in the mice described for FIG. 16A over time. Error bars represent standard error of the mean (SEM). Data are representative of two independent experiments.

FIGS. 17A and 17B: Potent in vitro anti-tumor activity of integrin αᵥβ₃ chimeric antigen receptor (CAR) T cells against triple negative breast cancer tumor cell lines MD-AMB-231 and HCC1143. FIG. 17A is a pair of graphs showing the anti-tumor cytotoxicity of CAR T cells in (top) MD-AMB-231 cells and (bottom) HCC1143 cells. αᵥβ₃.28z CAR T cells, αᵥβ₃.BBz CAR T cells, control CD19.28z CAR T cells or non-transduced T cells were co-cultured overnight in the breast cancer cell lines at various effector to target (E:T) cell ratios. Anti-tumor cytotoxicity was then evaluated by measuring electrical impedance of adherent tumor cells. FIG. 17B is a series of graphs showing levels (measured in picogram per milliliter (pg/ml)) of effector cytokines (interferon-gamma (IFN-γ, left-hand pair); tumor necrosis factor-alpha (TNF-α, center pair); and interleukin-2 (IL-2, right-hand pair)) in culture supernatants of MD-AMB-231 (top row) or HCC1143 (bottom row) triple negative breast cancer cells co-cultured with the T cells described for FIG. 17A for 24 hours at a 1:1 E:T ratio. Effector cytokine levels were measured by enzyme-linked immunosorbent assay (ELISA).

FIGS. 18A-18C: In vivo cytotoxicity of systemically administered integrin αᵥβ₃ chimeric antigen receptor (CAR) T cells against triple-negative breast tumors. FIG. 18A is a series of images of NOD scid gamma (NSG) immunodeficient mice with 1×10⁶ MD-AMB-231 tumor cells expressing green fluorescent protein (GFP)/firefly luciferase implanted into the fourth mammary pat pad via subcutaneous injection and, seven days later, with 5×10⁶ αᵥβ₃.28z CAR T cells, αᵥβ₃.BBz CAR T cells or control CD19.28z CAR T cells injected intravenously. Tumor burden in mice was monitored longitudinally via bioluminescent imaging and by caliper measurements. FIG. 18B is a graph showing the average tumor burden (radiance) in the mice described for FIG. 18A over time. FIG. 18C is a graph of the caliper measurements of palpable tumors in the mice described for FIG. 18A over time. Error bars represent standard error of the mean (SEM). Data are representative of two independent experiments. ****p<0.0001 determined by linear regression analysis of each growth curve. The slopes of each curve were then compared to determine if they were significantly different.

FIG. 19: Increased survival of mice engrafted with human triple-negative breast tumor cell line MD-AMB-231 following treatment with integrin αᵥβ₃ CAR T cells, but not control CD19 CAR T cells. Survival curves of mice treated intravenously with 5×10⁶ αᵥβ₃.28z CAR T cells, αᵥβ₃.BBz CAR T cells or control CD19.28z CAR T cells seven days after tumor cell implantation in the fourth mammary fat pad. Kaplan-Meier survival analysis was performed with Log-rank (Mantel-Cox) test for comparison between treatment groups.

FIG. 20: In vitro anti-tumor activity of integrin αᵥβ₃ chimeric antigen receptor (CAR) T cells against melanoma tumor cell lines. αᵥβ₃.28z CAR T cells, αᵥβ₃.BBz CAR T cells, control CD19.28z CAR T cells or non-transduced T cells were co-cultured overnight with SK-MEL-28, VMM18, DM93, DM13, SK-MEL-5 or VMM115 melanoma cell lines at various effector to target (E:T) cell ratios. Anti-tumor cytotoxicity in the SK-MEL-28 (top left), VMM18 (top right), DM93 (middle left), DM13 (middle right), SK-MEL-5 (bottom left), and VMM115 (bottom right) cells was evaluated by measuring electrical impedance of adherent tumor cells.

FIG. 21: In vivo cytotoxicity of systemically administered integrin αᵥβ₃ chimeric antigen receptor (CAR) T cells against human melanoma xenografts. NOD scid gamma (NSG) immunodeficient mice were implanted with 8×10⁶ SK-MEL-28 human melanoma tumor cells into the right flank via subcutaneous injection. Seven days later, 5×10⁶ αᵥβ₃.28z CAR, αᵥβ₃.BBz CAR, or control CD19.28z CAR T cells were injected intravenously. Tumor burden in mice was monitored by caliper measurements. Graphical representation of average tumor volume in mice over time is shown. Error bars represent standard error of the mean (SEM). Data are representative of two independent experiments. ****p<0.0001 determined by Two-ANOVA with Tukey's multiple comparisons test.

FIGS. 22A and 22B: In vitro anti-tumor activity of integrin αᵥβ₃ chimeric antigen receptor (CAR) T cells against rhabdomyosarcoma (RMS) tumor cell line RH18. αᵥβ₃.28z CAR T cells, αᵥβ₃.BBz CAR T cells, control CD19.28z CAR T cells or non-transduced T cells were co-cultured overnight with RMS RH18 cells at various effector to target (E:T) cell ratios. FIG. 22A is a graph showing anti-tumor cytotoxicity in the different treatment groups evaluated by measuring electrical impedance of adherent tumor cells. FIG. 22B shows the effector levels in culture supernatants harvested from RMS RH18 cancer cells co-cultured for 24 hours with αᵥβ₃.28z CAR T cells, αᵥβ₃.BBz CAR T cells, control CD19.28z CAR T cells or non-transduced T cells at a 1:1 E:T ratio. The graph at the left of FIG. 22B shows the levels (in picograms per milliliter (pg/ml)) of interferon-gamma (IFN-γ) and the graph at the right shows the levels of tumor necrosis factor-alpha (TNF-α) measured by enzyme-linked immunosorbent assay (ELISA).

FIGS. 23A and 23B: In vitro anti-tumor activity of integrin αᵥβ₃ chimeric antigen receptor (CAR) T cells against rhabdomyosarcoma (RMS) tumor cell line RH30. αᵥβ₃.28z CAR T cells, αᵥβ₃.BBz CAR T cells, control CD19.28z CAR T cells or non-transduced T cells were co-cultured overnight with RMS RH30 cells at various effector to target (E:T) cell ratios. FIG. 23A is a graph showing anti-tumor cytotoxicity in the different treatment groups evaluated by measuring electrical impedance of adherent tumor cells. FIG. 23B shows the effector levels in culture supernatants harvested from RMS RH30 cancer cells co-cultured for 24 hours with αᵥβ₃.28z CAR T cells, αᵥβ₃.BBz CAR T cells, control CD19.28z CAR T cells or non-transduced T cells at a 1:1 E:T ratio. The graph at the top left of FIG. 23B shows the levels (in picograms per milliliter (pg/ml)) of interferon-gamma (IFN-γ), the graph at the top right shows levels (in pg/ml) of interleukin-2 (IL-2), and the graph at the bottom shows the levels (in pg/ml) of tumor necrosis factor-alpha (TNF-α) measured by enzyme-linked immunosorbent assay (ELISA).

FIGS. 24A and 24B: In vitro anti-tumor activity of integrin αᵥβ₃ chimeric antigen receptor (CAR) T cells against cervical squamous cell carcinoma tumor cell line SW756. αᵥβ₃.28z CAR T cells, αᵥβ₃.BBz CAR T cells, control CD19.28z CAR T cells or non-transduced T cells were co-cultured overnight with SW756 cells at various effector to target (E:T) cell ratios. FIG. 24A is a graph showing anti-tumor cytotoxicity in the different treatment groups evaluated by measuring electrical impedance of adherent tumor cells. FIG. 24B shows the effector levels in culture supernatants harvested from SW756 cancer cells co-cultured for 24 hours with αᵥβ₃.28z CAR T cells, αᵥβ₃.BBz CAR T cells, control CD19.28z CAR T cells or non-transduced T cells at a 1:1 E:T ratio. The graph at the top left of FIG. 24B shows the levels (in picograms per milliliter (pg/ml)) of interferon-gamma (IFN-γ), the graph at the bottom shows levels (in pg/ml) of interleukin-2 (IL-2), and the graph at the top right shows the levels (in pg/ml) of tumor necrosis factor-alpha (TNF-α) measured by enzyme-linked immunosorbent assay (ELISA).

FIGS. 25A and 25B: In vitro anti-tumor activity of integrin αᵥβ₃ chimeric antigen receptor (CAR) T cells against bladder tumor cell line SW1710. αᵥβ₃.28z CAR T cells, αᵥβ₃.BBz CAR T cells, control CD19.28z CAR T cells or non-transduced T cells were co-cultured overnight with SW1710 cells at various effector to target (E:T) cell ratios. FIG. 25A is a graph showing anti-tumor cytotoxicity in the different treatment groups evaluated by measuring electrical impedance of adherent tumor cells. FIG. 25B shows the effector levels in culture supernatants harvested from SW1710 cancer cells co-cultured for 24 hours with αᵥβ₃.28z CAR T cells, αᵥβ₃.BBz CAR T cells, control CD19.28z CAR T cells or non-transduced T cells at a 1:1 E:T ratio. The graph at the top left shows the levels (in picograms per milliliter (pg/ml)) of interferon-gamma (IFN-γ), the graph at the bottom shows levels (in pg/ml) of interleukin-2 (IL-2), and the graph at the top right shows the levels (in pg/ml) of tumor necrosis factor-alpha (TNF-α) measured by enzyme-linked immunosorbent assay (ELISA).

FIGS. 26A and 26B: In vitro anti-tumor activity of integrin αᵥβ₃ chimeric antigen receptor (CAR) T cells against non-small cell lung cancer (NSCL) tumor cell line H1299. αᵥβ₃.28z CAR T cells, αᵥβ₃.BBz CAR T cells, control CD19.28z CAR T cells or non-transduced T cells were co-cultured overnight with H1299 cells at various effector to target (E:T) cell ratios. FIG. 26A is a graph showing anti-tumor cytotoxicity in the different treatment groups evaluated by measuring electrical impedance of adherent tumor cells. FIG. 26B shows the effector levels in culture supernatants harvested from H1299 cancer cells co-cultured for 24 hours with αᵥβ₃.28z CAR T cells, αᵥβ₃.BBz CAR T cells, control CD19.28z CAR T cells or non-transduced T cells at a 1:1 E:T ratio. The graph at the top shows the levels (in picograms per milliliter (pg/ml)) of interferon-gamma (IFN-γ) and the graph at the bottom shows levels (in pg/ml) of interleukin-2 (IL-2) measured by enzyme-linked immunosorbent assay (ELISA).

### DETAILED DESCRIPTION

The presently disclosed subject matter provides in some embodiments chimeric antigen receptor (CAR) T cells directed against integrin alphaᵥ beta₃ (αᵥβ₃), which is expressed on a variety of solid tumors as well as tumor vasculature, yet has minimal expression throughout normal tissues and organs.

More particularly, as described herein below, solid tumor cell lines were evaluated by flow cytometry for surface expression of integrin αᵥβ₃, particularly on lethal pediatric and adult brain tumor malignancies, such as diffuse intrinsic pontine glioma (DIPG) and glioblastoma (GBM). Second-generation CAR T cells expressing an anti-integrin αᵥβ₃ scFv were generated by retroviral transduction of exemplary CAR constructs containing either a CD28 or 4-1BB co-stimulatory domain and CD3zeta. CAR T cells were evaluated by flow cytometry for CAR expression, memory phenotype distribution, and inhibitory receptor profile. In vitro cytotoxic potential of integrin αᵥβ₃ CAR T cells was assessed by co-culture with tumor cells at various effector to target ratios and assayed with either bioluminescent- or impendence-based methods and effector cytokine production by ELISA. Multiple orthotopic xenograft studies, including brain tumor models for DIPG and GBM, were completed in NOD scid gamma (NSG) mice to evaluate the in vivo efficacy and safety of αᵥβ₃-targeted CAR T cells. DIPG and GBM cell lines were implanted into NSG mice via stereotactic injection and monitored with bioluminescent imaging to measure tumor burden following CAR T cell administration.

A majority of DIPG and multiple GBM cell lines tested positive for expression of integrin αᵥβ₃, as did a variety of other tumor cell lines, including breast, rhabdomyosarcoma, melanoma, lung, cervical, and bladder tumors. Tissue microarrays from normal healthy organs revealed little to no detectable integrin αᵥβ₃ expression. Healthy donor-derived PBMCs exhibited high level CAR expression in transduced T cells, efficient expansion, and representative populations of memory subsets including central, effector, and stem cell-like memory cells. αᵥβ₃.28z and αᵥβ₃.BBz CAR T cells exhibited in vitro cytotoxicity and effector cytokine production against all tumor types tested. αᵥβ₃.28z and αᵥβ₃.BBz CAR T cells mediated striking anti-tumor responses in vivo and were able to rapidly and significantly reduce established tumors in mice bearing orthotopic DIPG and GBM tumors, breast tumors and melanoma xenografts.

Thus, in some aspects, the presently disclosed subject matter provides a method of targeting integrins, particularly integrin αᵥβ₃, for anti-tumor activity. In some aspects, the presently disclosed subject matter highlights the broad applicability of utilizing CAR T cells targeting integrin αᵥβ₃ for immunotherapeutic treatment of multiple cancer types with reduced risk of on-target, off-tumor mediated toxicity due to the restricted nature of integrin αᵥβ₃ expression in normal tissues.

### I. Definitions

In describing and claiming the presently disclosed subject matter, the following terminology will be used in accordance with the definitions set forth below. The terminology used herein is for the purpose of describing particular embodiments only and is not intended to be limiting of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed subject matter and the claims.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In some embodiments, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds can also be used to treat symptoms associated with the injury, disease, or disorder, including, but not limited to, pain and inflammation.

As used herein, the term “adjuvant” refers to a substance that elicits an enhanced immune response when used in combination with a particular antigen.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three-letter code corresponding thereto, and/or by the one-letter code corresponding thereto, as summarized in Table 1:

TABLE 1
Amino Acids and Codes and Codons Therefor

| | 3- | 1- | Functionally |
|---|---|---|---|
| | Letter | Letter | Equivalent |
| Full Name | Code | Code | Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC |
| | | | CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC |
| | | | UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC |
| | | | CUG CUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the presently disclosed subject matter.

The term “amino acid” is used interchangeably with “amino acid residue” and can refer to a free amino acid and to an amino acid residue of a peptide. It will be apparent from the context in which the term is used whether it refers to a free amino acid or a residue of a peptide.

Amino acids have the following general structure:

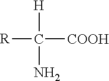

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

MicroRNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies or other methodologies.

An “antibody heavy chain”, as used herein, refers to the larger of the two types of polypeptide chains present in antibody molecules.

An “antibody light chain”, as used herein, refers to the smaller of the two types of polypeptide chains present in antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. By way of example and not limitation, an antigen can be derived from organisms, subunits of proteins/antigens, killed or inactivated whole cells or lysates.

The term “antimicrobial agent” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “basal medium”, as used herein, refers to a minimum essential type of medium, such as Dulbecco's Modified Eagle's Medium, Ham's F12, Eagle's Medium, RPMI, AR8, etc., to which other ingredients can be added. The term does not exclude media which have been prepared or are intended for specific uses, but which upon modification can be used for other cell types, etc.

The term “blastema”, as used herein, encompasses inter alia, the primordial cellular mass from which an organ, tissue or part is formed as well as a cluster of cells competent to initiate and/or facilitate the regeneration of a damaged or ablated structure.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

As used herein, the term “biologically active fragments” or “bioactive fragment” of the polypeptides encompasses natural or synthetic portions of the full-length protein that are capable of specific or selective binding to their natural ligand or of performing the function of the protein.

The term “biological sample”, as used herein, refers to any sample obtained from a subject, including, but not limited to, skin, hair, tissue, blood, plasma, cells, sweat and urine.

The term “bioresorbable”, as used herein, refers to the ability of a material to be resorbed in vivo. “Full” resorption means that no significant extracellular fragments remain.

The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable”, as the term is used herein, means that at least 80% of the total mass of material implanted is resorbed within one year.

The terms “cell” and “cell line”, as used herein, can be used interchangeably. All of these terms also include their progeny, which are any and all subsequent generations. It is understood that all progeny may not be identical due to deliberate or inadvertent mutations.

The terms “cell culture” and “culture”, as used herein, refer to the maintenance of cells in an artificial, in vitro environment. It is to be understood, however, that the term “cell culture” is a generic term and can be used to encompass the cultivation not only of individual cells, but also of tissues, organs, organ systems or whole organisms, for which the terms “tissue culture”, “organ culture”, “organ system culture” or “organotypic culture” can occasionally be used interchangeably with the term “cell culture”.

The phrases “cell culture medium”, “culture medium” (plural “media” in each case) and “medium formulation” refer to a nutritive solution for cultivating cells and can be used interchangeably.

The term “cell surface protein” means a protein found where at least part of the protein is exposed at the outer aspect of the cell membrane. Examples include growth factor receptors.

As used herein, the term “chemically conjugated”, or “conjugating chemically” refers to linking of one chemical entity to another chemical entity, for example an antigen to a carrier molecule. This linking can occur on the genetic level using recombinant technology, wherein a hybrid protein can be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds can also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and in some embodiments at least about 75%, at least about 90%, or at least about 95% of the nucleotide residues of the first portion are capable of base pairing with nucleotide residues in the second portion. More in some embodiments, all nucleotide residues of the first portion are capable of base pairing with nucleotide residues in the second portion.

A “compound”, as used herein, refers to any type of substance or agent that is commonly considered a drug, or a candidate for use as a drug, combinations, and mixtures of the above, as well as polypeptides and antibodies of the presently disclosed subject matter.

A “computer-readable medium” is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information can be transferred between a system of interest and a medium, between computers, or between computers and the computer-readable medium for storage or access of stored information. Such transmission can be electrical, or by other available methods, such as IR links, wireless connections, etc.

A “conditioned medium” is one prepared by culturing a first population of cells or tissue in a medium, and then harvesting the medium. The conditioned medium (along with anything secreted into the medium by the cells) can then be used to support the growth or differentiation of a second population of cells.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

The term “delivery vehicle” refers to any kind of device or material which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), can be different from when that compound is administered alone. Thus, an effective amount of a combination of compounds refers collectively to the combination as a whole, although the actual amounts of each compound can vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and in some embodiments at least about 99%, by weight, of the protein or peptide in the preparation is the particular protein or peptide.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, “feeders”, and “feeder layers” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, can ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Graft” refers to any free (unattached) cell, tissue, or organ for transplantation.

“Autologous” as used herein, refers to cells from the same subject.

“Allogeneic” as used herein, refers to cells of the same species.

“Xenograft” or “xenogeneic” refers to a transplanted cell, tissue, or organ derived from an animal of a different species.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β) platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors can also promote differentiation of a cell or tissue. TGF, for example, can promote growth and/or differentiation of a cell or tissue. Note that many factors are pleiotropic in their activity and the activity can vary depending on things such as the cell type being contacted, the state of proliferation or differentiation of the cell, etc.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the)(BLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

Identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 MNaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds.

Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit”, as used herein, means to suppress or block an activity or function such that it is lower relative to a control value. The inhibition can be via direct or indirect mechanisms. In some embodiments, the activity is suppressed or blocked by at least 10% compared to a control value, more in some embodiments by at least 25%, and in some embodiments by at least 50%.

The term “inhibitor” as used herein, refers to any compound or agent, the application of which results in the inhibition of a process or function of interest, including, but not limited to, expression, levels, and activity. Inhibition can be inferred if there is a reduction in the activity or function of interest.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound or cells of the presently disclosed subject matter by any number of routes and means including, but not limited to, intravitreal, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

The term “injury” refers to any physical damage to the body caused by violence, accident, trauma, or fracture, etc., as well as damage by surgery.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material can describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material can be shipped separately from the container with the intention that the instructional material and the compound be used cooperatively by the recipient.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats can be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression can cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample can of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “progeny” of a stem cell as used herein refers to a cell which is derived from a stem cell and can still have all of the differentiation abilities of the parental stem cell, i.e., multipotency, or one that can no longer be multipotent, but is now committed to being able to differentiate into only one cell type, i.e., a committed cell type. The term can also refer to a differentiated cell.

The term “peptide” typically refers to short polypeptides.

The term “per application” as used herein refers to administration of cells, a drug, or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide can be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but can be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence can be the core promoter sequence and in other instances, this sequence can also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

The term “propagate” means to reproduce or to generate.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. Representative purification techniques are disclosed herein for antibodies and fragments thereof.

“Recombinant polynucleotide” refers to a polynucleotide having sequences that are not naturally joined together. An amplified or assembled recombinant polynucleotide can be included in a suitable vector, and the vector can be used to transform a suitable host cell.

A recombinant polynucleotide can serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some embodiments, scFv are expressed on the surface of an engineered cell.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” when used in reference to a substrate forming a linkage with a compound, relates to a solvent insoluble substrate that is capable of forming linkages (in some embodiments covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “solid support suitable for maintaining cells in a tissue culture environment” is meant any surface such as a tissue culture dish or plate, or even a cover, where medium containing cells can be added, and that support can be placed into a suitable environment such as a tissue culture incubator for maintaining or growing the cells. This should of course be a solid support that is either sterile or capable of being sterilized. The support does not need to be one suitable for cell attachment.

The term “solid support is a low adherence, ultralow adherence, or non-adherence support for cell culture purposes” refers to a vehicle such as a bacteriological plate or a tissue culture dish or plate which has not been treated or prepared to enhance the ability of mammalian cells to adhere to the surface. It could include, for example, a dish where a layer of agar has been added to prevent cells from attaching. It is known to those of ordinary skill in the art that bacteriological plates are not treated to enhance attachment of mammalian cells because bacteriological plates are generally used with agar, where bacteria are suspended in the agar and grow in the agar.

The term “standard”, as used herein, refers to something used for comparison. For example, a standard can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, more in some embodiments by at least 25%, and in some embodiments by at least 50%.

The term “stimulator” as used herein, refers to any composition, compound or agent, the application of which results in the stimulation of a process or function of interest, including, but not limited to, wound healing, angiogenesis, bone healing, osteoblast production and function, and osteoclast production, differentiation, and activity.

A “subject” of diagnosis or treatment is an animal, including a human. It also includes pets and livestock.

As used herein, a “subject in need thereof” is a patient, animal, mammal, or human, who will benefit from the method of this presently disclosed subject matter.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “substantially pure” describes a compound, e.g., a protein or polypeptide which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “substituent” as used in the phrase “other cells which are not substituents of the at least one self-organizing blastema” refers to substituent cells of the blastema. Therefore, a cell which is not a substituent of a self-organizing blastema can be a cell that is adjacent to the blastema and need not be a cell derived from a self-organizing blastema.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The use of the phrase “tissue culture dish or plate” refers to any type of vessel which can be used to plate cells for growth or differentiation.

The term “thermal injury” is used interchangeably with “thermal burn” herein.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transduction” is used interchangeably with the terms “gene transfer”, “transformation”, and “transfection”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, lentiviruses, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods useful for the practice of the presently disclosed subject matter which are not described herein are also known in the art. Useful methods include those described in PCT International Patent Application Publication Nos. WO 2007/019107; WO 2007/030652; WO 2007/089798; WO 2008/060374, the methods of which are hereby incorporated by reference.

### II. Anti-αᵥβ₃ Cars and Related Compositions

The presently disclosed subject matter provides, in some embodiments, a cell (e.g., an immune cell such as a T cell, a natural killer (NK) cell or NK-like T cell (NKT cell)) engineered to express a CAR, wherein the CAR-expressing cell exhibits an antitumor property and can bind the integrin complex αᵥβ₃. In some embodiments, the presently disclosed subject matter provides a polypeptide comprising a CAR, wherein the CAR comprises an antigen-binding domain that binds integrin αᵥβ₃. In some embodiments, the presently disclosed subject matter provides a nucleic acid (e.g., an isolated nucleic acid, a nucleic acid construct, or a vector) encoding the CAR that binds integrin αᵥβ₃.

In some embodiments, the CAR comprises an extracellular domain, a transmembrane domain, and an intracellular (or cytoplasmic) domain. The extracellular domain can comprise a target-specific binding element otherwise referred to as an antigen-binding domain. The intracellular domain (or cytoplasmic domain) comprises at least one signaling domain and at least one costimulatory (CS) signaling region. The costimulatory signaling region refers to a portion of the CAR comprising the intracellular domain of a costimulatory molecule. Costimulatory molecules are cell surface molecules other than antigens receptors or their ligands that are involved in an efficient response of lymphocytes to antigen. Accordingly, in some embodiments, the presently disclosed subject matter provides a CAR, wherein the CAR comprises an αᵥβ₃ binding domain, a transmembrane domain, at least one cytoplasmic CS domain, and an intracellular signaling domain. In some embodiments, the presently disclosed CAR is a fully human CAR.

In some embodiments, the presently disclosed subject matter provides an isolated nucleic acid or a nucleic acid construct comprising sequences encoding a CAR, wherein the sequence comprises the nucleic acid sequence of an antigen binding moiety operably linked to the nucleic acid sequence of an intracellular domain. An exemplary intracellular domain that can be used in the CAR of the presently disclosed subject matter includes but is not limited to the intracellular domain of CD3-zeta (CD3ζ), CD27, CD28, and the like. In some instances, the CAR can comprise a combination of (1) CD3-zeta and (2) CD28 and/or 4-1BB and the like. In some embodiments, the presently disclosed subject matter provides an isolated nucleic acid encoding a CAR, wherein the CAR comprises an αᵥβ₃ binding domain, a transmembrane domain, a cytoplasmic co-stimulatory (CS) domain, and an intracellular signaling domain.

Between the extracellular domain and the transmembrane domain or between the cytoplasmic domain and the transmembrane domain, the CAR can optionally comprise a spacer domain. As used herein, the term “spacer domain” generally means any oligo- or polypeptide that functions to link the transmembrane domain to, either the extracellular domain or, the cytoplasmic domain in the polypeptide chain. In some embodiments, the spacer domains can comprise up to about 300 amino acids. In some embodiments, the spacer comprises 10 to 100 amino acids or 25 to 50 amino acids. In some embodiments, the CAR is free of a spacer domain between the extracellular domain and the transmembrane domain. In some embodiments, the CAR is free of a spacer comprising an IgG4 hinge region.

Generally, the antigen binding domain of the CAR antigen binding domain can be any domain that binds to αᵥβ₃ including, but not limited to, monoclonal antibodies, polyclonal antibodies, synthetic antibodies, human antibodies, humanized antibodies, and fragments thereof. In some embodiments, it is beneficial for the antigen binding domain to be derived from the same species in which the CAR will ultimately be used in. For example, for use in humans, it can be beneficial for the antigen binding domain of the CAR to comprise a human antibody or fragment thereof. Thus, in some embodiments, the antigen biding domain portion comprises a human antibody or a fragment thereof. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Human antibodies can also be produced using transgenic mice which are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the presently disclosed subject matter. Anti-αᵥβ₃ antibodies directed against the human αᵥβ₃ can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. For a discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies can also be generated by in vitro activated B cells (see, U.S. Pat. Nos. 5,567,610 and 5,229,275, each of which is incorporated herein by reference in its entirety). Human antibodies can also be generated in vitro using hybridoma techniques such as, but not limited to, that described by Roder et al. (Methods Enzymol, 121:140-167 (1986)).

Alternatively, in some embodiments, a non-human antibody is humanized, where specific sequences or regions of the antibody are modified to increase similarity to an antibody naturally produced in a human. In some embodiments, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), and chain shuffling (see, e.g., U.S. Pat. No. 5,565,332, which is incorporated herein in its entirety by reference). Often, framework residues in the framework regions can be substituted with the corresponding residue from the CDR donor antibody to alter (e.g., improve) antigen binding. These framework substitutions are identified by methods well-known in the art, e.g., by modeling of the interactions of the CDR and framework residues to identify framework residues important for antigen binding and sequence comparison to identify unusual framework residues at particular positions. See, e.g., Queen et al., U.S. Pat. No. 5,585,089, which is incorporated herein by reference in its entirety.

A humanized antibody can have one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. In practice, humanized antibodies are typically human antibodies in which some CDR residues and possibly some framework (FR) residues are substituted by residues from analogous sites in rodent antibodies. In some instances, a human scFv can also be derived from a yeast display library.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (see e.g., Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework can be used for several different humanized antibodies (see e.g., Carter et al., Proc. Natl. Acad. Sci. USA, 89:4285 (1992); Presta et al., J. Immunol., 151:2623 (1993).

Antibodies can be humanized with retention of high affinity for the target antigen and other favorable biological properties. In some embodiments, antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A “humanized” antibody retains a similar antigenic specificity as the original antibody, i.e., in the presently disclosed subject matter, the ability to bind human integrin αᵥβ₃. However, using certain methods of humanization, the affinity and/or specificity of binding of the antibody for integrin αᵥβ₃ can be increased using methods of “directed evolution,” as described, for example, by Wu et al., J. Mol. Biol., 294:151 (1999).

In some embodiments, the antigen binding moiety portion in the CAR is an anti-integrin αᵥβ₃ scFV (e.g., a human anti-αᵥβ₃ scFV). In some embodiments, the scFV comprises sequences from a framework optimized version of the monoclonal antibody LM609 (NCBI accession number: NM_002210.2; UniProt accession number: P06756), which comprises a light chain variable region (V_{L}) of the sequence ELVMTQTPATLSVTPGDSVSLSCRASQSISNHLHWYQQKSHESPRLLIKYASQSIS GIPSRFSGSGSGTDFTLSINSVETEDFGMYFCQQSNSWPHTFGGGTKLEIK (SEQ ID NO: 22) and a heavy chain variable region (V_{H}) of the sequence EVQLEESGGGLVKPGGSLKLSCAASGFAFSSYDMSWVRQIPEKRLEWVAKVSSG GGSTYYLDTVQGRFTISRDNAKNTLYLQMSSLNSEDTAMYYCARHNYGSFAYW GQGTLVTVSA (SEQ ID NO: 23). In some embodiments, the presently disclosed CAR comprises a scFV comprising a V_{H} comprising, consisting essentially of, or consisting of SEQ ID NO: 13 or an amino acid sequence that is at least 70%, 75%, 80%, 85%, 90%, 91%, 92%, 93%, 94%, 95%, 96%, 97%, 98%, 99% or more identical thereto. In some embodiments, the scFV comprises a V_{L} comprising, consisting essentially of, or consisting of SEQ ID NO: 15 or an amino acid sequence that is at least 70%, 75%, 80%, 85%, 90%, 91%, 92%, 93%, 94%, 95%, 96%, 97%, 98%, 99% or more identical thereto. In some embodiments, the CAR comprises a V_{H} comprising complementarity determining regions CDR-VH1, CDR-VH2, and CDR-VH3 of the V_{H} of SEQ ID NO: 13, wherein CDR-VH1, CDR-VH2, and CDR-VH3 comprise, consist essentially of, or consist of amino acids 31-35, 50-66, and 99-106 of SEQ ID NO: 13, respectively. In some embodiments, the CAR comprises a V_{L} comprising complementarity determining regions CDR-VL1, CDR-VL2, and CDR-VL3 of the V_{L} of SEQ ID NO: 15, wherein CDR-VL1, CDR-VL2, and CDR-VL3 comprise, consist essentially of, or consist of amino acids 24-34, 50-56, and 88-97 of SEQ ID NO: 15, respectively.

In some embodiments, the scFV comprises a linker peptide located between SEQ ID NO: 13 and SEQ ID NO: 15. In some embodiments, the linker peptide comprises or consists of glycine and serine residues or repeating subsequences comprising or consisting of glycine and serine. In some embodiments, the linker peptide comprises, consists essentially of, or consists of SEQ ID NO: 14 or an amino acid sequence that is at least 70%, 75%, 80%, 85%, 90%, 91%, 92%, 93%, 94%, 95%, 96%, 97%, 98%, 99% or more identical thereto.

In some embodiments, the scFV comprises a V_{H} encoded by a nucleic acid sequence comprising, consisting essentially of, or consisting of SEQ ID NO: 3 or a nucleic acid sequence that is at least 50%, 55%, 60%, 65%, 70%, 75%, 80%, 85%, 90%, 91%, 92%, 93%, 94%, 95%, 96%, 97%, 98%, 99% or more identical thereto. In some embodiments, the scFV comprises a V_{L} encoded by a nucleic acid sequence comprising, consisting essentially of, or consisting of SEQ ID NO: 5 or a nucleic acid sequence that is at least 50%, 55%, 60%, 65%, 70%, 75%, 80%, 85%, 90%, 91%, 92%, 93%, 94%, 95%, 96%, 97%, 98%, 99% or more identical thereto. In some embodiments, the scFV comprises a linker peptide located between the V_{H} and V_{L} that is encoded by a nucleic acid that comprises or consists of SEQ ID NO: 4 or a nucleic acid sequence that is at least 50%, 55%, 60%, 65%, 70%, 75%, 80%, 85%, 90%, 91%, 92%, 93%, 94%, 95%, 96%, 97%, 98%, 99% or more identical thereto.

In some embodiments, the anti-αᵥβ₃ CAR comprises an anti-integrin αᵥβ₃ scFV and (1) the scFV comprises a V_{H} comprising or consisting of SEQ ID NO: 13 or a sequence at least 90% identical thereto or a sequence comprising CDRs corresponding to one, two, or three of the CDRs of the V_{H} of SEQ ID NO: 13 (i.e., sequences corresponding to amino acids 31-35, 50-66, and 99-106 of SEQ ID NO: 13) and a V_{L} comprising or consisting of SEQ ID NO: 15 or a sequence at least 90% identical thereto or a sequence comprising CDRs corresponding to one, two or three of the CDRs of the V_{L} of SEQ ID:15 (i.e., sequences corresponding to amino acids 24-34, 50-56, and 88-97 of SEQ ID NO: 15); and/or (2) the CAR is free of an extracellular IgG4 hinge domain. In some embodiments, the isolated nucleic acid encodes a CAR comprising an anti-αᵥβ₃ scFV and (1) the scFV comprises a V_{H} encoded by SEQ ID NO: 3 or by a sequence at least 90% identical to SEQ ID NO: 3 and a V_{L} encoded by SEQ ID NO: 5 or by a sequence at least 90% identical to SEQ ID NO: 5; and/or (2) the CAR is free of an extracellular IgG4 hinge domain.

With respect to the transmembrane domain, the CAR can be designed to comprise a transmembrane domain that is fused to the extracellular domain of the CAR. In some embodiments, the transmembrane domain that naturally is associated with one of the domains in the CAR can be used. In some instances, the transmembrane domain can be selected or modified by amino acid substitution to avoid binding of such domains to the transmembrane domains of the same or different surface membrane proteins to minimize interactions with other members of the receptor complex.

The transmembrane domain can be derived either from a natural or from a synthetic source. Where the source is natural, the domain can be derived from any membrane-bound or transmembrane protein. Suitable transmembrane regions include, but are not limited to the transmembrane region(s) of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, a hinge from a human immunoglobulin can be used. In some embodiments, the CAR does not comprise a hinge from an IgG4. In some embodiments, the transmembrane region does not comprise a hinge of a human immunoglobulin.

In some embodiments, the transmembrane domain is synthetic, in which case it can comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length can form the linkage between the transmembrane domain and the cytoplasmic signaling domain of the CAR.

In some embodiments, the transmembrane region comprises the transmembrane region of CD8 alpha. In some embodiments, the transmembrane region comprises or consists of SEQ ID NO: 17 or an amino acid sequence at least 70%, 75%, 80%, 85%, 90%, 91%, 92%, 93%, 94%, 95%, 96%, 97%, 98%, 99% or more identical thereto. In some embodiments, the transmembrane region is encoded by a nucleic acid comprising or consisting of SEQ ID NO: 7 or a nucleic acid sequence that is at least 50%, 55%, 60%, 65%, 70%, 75%, 80%, 85%, 90%, 91%, 92%, 93%, 94%, 95%, 96%, 97%, 98%, 99% or more identical thereto.

The cytoplasmic domain of the CAR is responsible for activation of at least one of the normal effector functions of an immune cell in which the CAR has been placed. The term “effector function” refers to a specialized function of a cell. Effector function of a T cell, for example, can be cytolytic activity or helper activity including the secretion of cytokines. Thus, the term “intracellular signaling domain” refers to the portion of a protein which transduces the effector function signal and directs the cell to perform a specialized function. In some embodiments, the entire intracellular signaling domain can be employed, however in some embodiments, less than the entire intracellular signaling domain is used. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the presently disclosed subject matter include, for example, the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone can be insufficient for full activation of the T cell. Thus, a secondary or co-stimulatory signal can be employed. Thus, T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic (or intracellular) signaling sequences regulate primary activation of the TCR complex either in a stimulatory way, or in an inhibitory way. Primary intracellular signaling sequences that act in a stimulatory manner can contain signaling motifs which are known as immunoreceptor tyrosine-based activation motifs or ITAMs.

Examples of ITAM containing primary intracellular signaling sequences that are of use in the presently disclosed subject matter include those derived from TCR zeta, FcR gamma, FcR beta, CD3 gamma, CD3 delta, CD3 epsilon, CD5, CD22, CD79a, CD79b, and CD66d. In some embodiments, the cytoplasmic signaling molecule in the CAR of the presently disclosed subject matter comprises an intracellular signaling sequence derived from CD3 zeta.

In some embodiments, the CAR comprises an intracellular signaling domain comprising or consisting of SEQ ID NO: 20 or an amino acid sequence that is at least 70%, 75%, 80%, 85%, 90%, 91%, 92%, 93%, 94%, 95%, 96%, 97%, 98%, 99% or more identical thereto. In some embodiments, the CAR comprises an intracellular signaling domain encoded by a nucleic acid comprising or consisting of SEQ ID NO: 10 or a nucleic acid sequence that is at least 50%, 55%, 60%, 65%, 70%, 75%, 80%, 85%, 90%, 91%, 92%, 93%, 94%, 95%, 96%, 97%, 98%, 99% or more identical thereto.

In some embodiments, the cytoplasmic (or intracellular) domain of the CAR can comprise a CD3 zeta chain portion and at least one costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include, but are not limited to, CD27, CD28, 4-1BB (CD137 or tumor necrosis factor superfamily member 9), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

In some embodiments, the cytoplasmic co-stimulatory (CS) domain comprises one or both of a CD28 signaling domain and a 4-1BB signaling domain. In some embodiments, the CS domain comprises or consists of a CD28 signaling domain. In some embodiments, the CS domain comprises or consists of a 4-1BB signaling domain. In some embodiments, the CD28 signaling domain comprises or consists of SEQ ID NO: 18 or an amino acid sequence at least 70%, 75%, 80%, 85%, 90%, 91%, 92%, 93%, 94%, 95%, 96%, 97%, 98%, 99% or more identical thereto. In some embodiments, the CD28 signaling domain is encoded by a nucleic acid sequence comprising or consisting of SEQ ID NO: 8 or a nucleic acid sequence at least 50%, 55%, 60%, 65%, 70%, 75%, 80%, 85%, 90%, 91%, 92%, 93%, 94%, 95%, 96%, 97%, 98%, 99% or more identical thereto. In some embodiments, the 4-1BB signaling domain comprises or consists of SEQ ID NO: 19 or an amino acid sequence at least 70%, 75%, 80%, 85%, 90%, 91%, 92%, 93%, 94%, 95%, 96%, 97%, 98%, 99% or more identical thereto. In some embodiments, the 4-1BB signaling domain is encoded by a nucleic acid sequence comprising or consisting of SEQ ID NO: 9 or a nucleic acid sequence at least 50%, 55%, 60%, 65%, 70%, 75%, 80%, 85%, 90%, 91%, 92%, 93%, 94%, 95%, 96%, 97%, 98%, 99% or more identical thereto.

The cytoplasmic signaling sequences within the cytoplasmic signaling portion of the CAR of the presently disclosed subject matter can be linked to each other in a random or specified order. Optionally, a short oligo- or polypeptide linker, preferably between 2 and 10 amino acids in length can form the linkage.

In some embodiments, the CAR of the presently disclosed subject matter further comprises an extracellular signal peptide, e.g., attached directly to the antigen binding region. In some embodiments, the signal peptide is a granulocyte-macrophage colony-stimulating factor (GM-CSF) signal peptide. In some embodiments, the GM-CSF signal peptide comprises or consists of SEQ ID NO: 12 or an amino acid sequence that is at least 70%, 75%, 80%, 85%, 90%, 91%, 92%, 93%, 94%, 95%, 96%, 97%, 98%, 99% or more identical thereto. In some embodiments, the GM-CSF signal peptide is encoded by a nucleic acid sequence comprising or consisting of SEQ ID NO: 2 or a nucleic acid sequence that is at least 50%, 55%, 60%, 65%, 70%, 75%, 80%, 85%, 90%, 91%, 92%, 93%, 94%, 95%, 96%, 97%, 98%, 99% or more identical thereto.

In some embodiments, the CAR comprises one or more additional sequences, e.g., restriction enzyme sites, spacers, transduction markers, etc.

In some embodiments, the presently disclosed CAR comprises, consists essentially of, or consists of, in the following order, from N-terminus to C-terminus: (i) SEQ ID NO: 12; (ii) SEQ ID NO: 13; (iii) SEQ ID NO: 14; (iv) SEQ ID NO: 15; (v) SEQ ID NO: 16; (vi) SEQ ID NO: 17; (vii) SEQ ID NO: 18 and/or SEQ ID NO: 19; and (vii) SEQ ID NO: 20. In some embodiments, (vii) comprises both SEQ ID NO: 18 and SEQ ID NO: 19. In some embodiments, (vii) comprises SEQ ID NO: 18 or SEQ ID NO: 19. In some embodiments, (vii) comprises SEQ ID NO: 18. In some embodiments, (vii) comprises SEQ ID NO: 19. In some embodiments, one or more of the sequences can be replaced by a sequence that is at least 70% identical to the recited sequence (e.g., the sequence of SEQ ID NO: 13 can be replaced by a sequence that is at least 70% identical to SEQ ID NO: 13). In some embodiments, one or more of the sequence can be replaced by a sequence that is 75%, 80%, 85%, 86%, 87%, 88%, 89%, 90%, 91%, 92%, 93%, 94%, 95%, 96%, 97%, 98%, or 99% or more identical to the recited sequence.

In some embodiments, the presently disclosed subject matter provides an isolated nucleic acid encoding the CAR, wherein the nucleic acid comprises, consists essentially of, or consists of, in the following order: (i) SEQ ID NO: 2; (ii) SEQ ID NO: 3; (iii) SEQ ID NO: 4; (iv) SEQ ID NO: 5; (v) SEQ ID NO: 6; (vi) SEQ ID NO: 7; (vii) SEQ ID NO: 8 and/or SEQ ID NO: 9; and (vii) SEQ ID NO: 10. In some embodiments, (vii) comprises both SEQ ID NO: 8 and SEQ ID NO: 9. In some embodiments, (vii) comprises SEQ ID NO: 8 or SEQ ID NO: 9. In some embodiments, (vii) comprises SEQ ID NO: 8. In some embodiments, (vii) comprises SEQ ID NO: 9. In some embodiments, one or more of the sequences can be replaced by a sequence that is at least 70% identical to the recited sequence (e.g., the sequence of SEQ ID NO: 3 can be replaced by a sequence that is at least 70% identical to SEQ ID NO: 3). In some embodiments, one or more of the sequence can be replaced by a sequence that is 75%, 80%, 85%, 86%, 87%, 88%, 89%, 90%, 91%, 92%, 93%, 94%, 95%, 96%, 97%, 98%, 99% or more identical to the recited sequence.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The presently disclosed subject matter further encompasses methods and compositions for inserting the CAR or CAR encoding nucleic acid of the presently disclosed subject matter into a cell. Thus, the presently disclosed subject matter relates to vectors and non-vector delivery systems, such as, but not limited to lentiviral, adenoviral, sleeping beauty transposon/transposase. CRISPR/cas9 methods and systems of inserting the CAR encoding nucleic acid into a cell's genome or otherwise causing it to be expressed (including transiently expressed) in a cell (e.g., via mRNA transfection).

In some embodiments, the presently disclosed subject matter provides a vector in which a nucleic acid of the presently disclosed subject matter is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the desired CAR can be expressed in the cells by way of transponsons.

In brief summary, the expression of natural or synthetic nucleic acids encoding CARs is typically achieved by operably linking a nucleic acid encoding the CAR polypeptide or portions thereof to a promoter and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The expression constructs of the presently disclosed subject matter can also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the presently disclosed subject matter provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Exemplary vectors include, but are not limited to, expression vectors, replication vectors, probe generation vectors, and sequencing vectors.

In some embodiments, the expression vector can be provided to a cell in the form of a viral vector. Viral vector technology is well known in the art and is described, for example, in Sambrook et al. (2001) Molecular Cloning: A Laboratory Manual, Cold Spring Harbor Laboratory, New York, and in other virology and molecular biology manuals. Viruses, which are useful as vectors include, but are not limited to, retroviruses, adenoviruses, adeno-associated viruses, herpes viruses, and lentiviruses.

For example, retroviruses provide a convenient platform for delivery systems. A selected gene can be inserted into a vector and packaged in retroviral particles using techniques known in the art. The recombinant virus can then be isolated and delivered to cells of the subject either in vivo or ex vivo. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), Spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193). Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α. (EF-1α). However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the presently disclosed subject matter should not be limited to the use of constitutive promoters. Inducible promoters are also contemplated as part of the presently disclosed subject matter. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker can be carried on a separate piece of DNA and used in a co-transfection procedure. Both selectable markers and reporter genes can be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes can include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and can be prepared using known techniques or obtained commercially. In general, the construct with the minimal 5′ flanking region showing the highest level of expression of reporter gene is identified as the promoter. Such promoter regions can be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001), Molecular Cloning: A Laboratory Manual, Cold Spring Harbor Laboratory, New York.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid can be associated with a lipid. The nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo., United States of America; dicetyl phosphate (“DCP”) can be obtained from K & K Laboratories (Plainview, N.Y., United States of America); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala., United States of America). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids cab assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays can be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the presently disclosed subject matter.

In some embodiments, the genetically modified immune cells of the presently disclosed subject matter are modified through the introduction of RNA. In some embodiments, an in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is the CAR of the presently disclosed subject matter. For example, the template for the RNA CAR comprises an extracellular domain comprising an anti-αᵥβ₃ scFv; a transmembrane domain comprising the hinge and transmembrane domain of CD8a; and a cytoplasmic domain comprises the signaling domain of CD3-zeta and CD28 and/or 4-1BB CS domains.

In some embodiments, the DNA to be used for PCR contains an open reading frame. The DNA can be from a naturally occurring DNA sequence from the genome of an organism. In some embodiments, the DNA is a full length gene of interest of a portion of a gene. The gene can include some or all of the 5′ and/or 3′ untranslated regions (UTRs). The gene can include exons and introns. In some embodiments, the DNA to be used for PCR is a human gene. In some embodiments, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that provide a therapeutic or prophylactic effect to an organism or that can be used to diagnose a disease or disorder in an organism. Preferred genes are genes which are useful for a short term treatment, or where there are safety concerns regarding dosage or the expressed gene. For example, for treatment of cancer, the transgene(s) to be expressed can encode a polypeptide that functions as a ligand or receptor for cells of the immune system, or can function to stimulate or inhibit the immune system of an organism. In some embodiments, it is not desirable to have prolonged ongoing stimulation of the immune system, nor necessary to produce changes which last after successful treatment.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency can also be used. The RNA preferably has 5′ and 3′ UTRs. In some embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or to translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be needed for all RNAs to enable efficient translation. The requirement for Kozak sequences for many mRNAs is known in the art. In some embodiments the 5′ UTR can be derived from an RNA virus whose RNA genome is stable in cells. In some embodiments, various nucleotide analogues can be used in the 3′ or 5′ UTR to impede exonuclease degradation of the mRNA.

To provide synthesis of RNA from a DNA template without the need for gene cloning, a promoter of transcription can be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. Thus, a method which allows construction of DNA templates with polyA/T 3′ stretch without cloning are highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence can be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa et al. Hum Gene Ther., 12(8):861-70 (2001)).

In some embodiments, the presently disclosed subject matter provides a vector or non-vector delivery system comprising a nucleic acid encoding a CAR of the presently disclosed subject matter. In some embodiments, the presently disclosed subject matter provides a vector or a kit of vectors encoding the CAR and/or comprising the isolated nucleic acid described hereinabove. Such a vector or delivery system can be used to introduce the nucleic acid sequence into a host cell so that it expresses a CAR according to the presently disclosed subject matter. In some embodiments, the vector is a plasmid, a viral vector, a transposon-based vector, or synthetic mRNA. In some embodiments, the vector is a viral vector. In some embodiments, the viral vector is a retroviral vector or a lentiviral vector. In some embodiments, the vector is a retroviral vector. In some embodiments, the vector can be capable of transfecting or transducing an immune cell (e.g., a T cell, a NK cell or a natural killer-like T (NKT) cell).

In some embodiments, the above-presented amino acid sequences can be modified; for example, where only changes not significantly affecting the peptide function occur. Modifications can be accomplished by any suitable approach, such as but not limited to by modifying a nucleic acid sequence encoding the peptide.

As used herein an “amino acid modification” refers in some embodiments to a substitution, addition, or deletion of an amino acid, and includes substitution with, or addition of, any of the 20 amino acids commonly found in human proteins, as well as unusual or non-naturally occurring amino acids such as but not limited to D-amino acids. Commercial sources of unusual amino acids include Sigma-Aldrich (Milwaukee, Wis., United States of America), ChemPep Inc. (Miami, Fla., United States of America), and Genzyme Pharmaceuticals (Cambridge, Mass., United States of America). Unusual amino acids can be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid. The term “modified peptide” encompasses any amino acid modification as described herein.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substitutions can be designed based on, for example, the model of Dayhoff et al. (in Atlas of Protein Sequence and Structure 1978, National Biomedical Research Foundation, Washington D.C., United States of America).

In some embodiments, an amino acid substitution is a conservative amino acid substitution. As used herein, the term “conservative amino acid substitution” is defined in some embodiments as exchanges within one of the following five groups:

I. Small aliphatic, nonpolar or slightly polar residues: Ala, Ser, Thr, Pro, Gly;

II. Polar, charged residues and their amides: Asp, Asn, Glu, Gln, His, Arg, Lys;

III. Large, aliphatic, nonpolar residues: Met Leu, Ile, Val, Cys

IV. Large, aromatic residues: Phe, Tyr, Trp

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al. (1990) Science 247:1306-1310.

For example, the hydropathic index of amino acids may be considered (Kyte & Doolittle (1982) J Mol Biol 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle (1982) J Mol Biol 157:105-132), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet, or reverse turn secondary structure has been determined and is known in the art (see e.g., Chou & Fasman (1974) Biochemistry 13:222-245; Chou & Fasman (1978) Ann Rev Biochem 47: 251-276; Chou & Fasman (1979) Biophys J 26:367-384).

Based on such considerations and extensive empirical study, tables of conservative amino acid substitutions have been constructed and are known in the art. By way of example and not limitation, the following substitutions can be made: arginine and lysine; glutamate and aspartate; serine and threonine; glutamine and asparagine; and valine, leucine, and isoleucine. Alternatively, Table 2 lists exemplary conservative amino acid substitutions.

TABLE 2
Exemplary Conservative Amino Acid Substitutions

| Amino Acid | Possible Substitution(s) |
|---|---|
| Ala (A) | Leu, Ile, Val |
| Arg (R) | Gln, Asn, Lys |
| Asn (N) | His, Asp, Lys, Arg, Gln |
| Asp (D) | Asn, Glu |
| Cys (C) | Ala, Ser |
| Gln (Q) | Glu, Asn |
| Glu (E) | Gln, Asp |
| Gly (G) | Ala |
| His (H) | Asn, Gln, Lys, Arg |
| Ile (I) | Val, Met, Ala, Phe, Leu |
| Leu (L) | Val, Met, Ala, Phe, Ile |
| Lys (K) | Gln, Asn, Arg |
| Met (M) | Phe, Ile, Leu |
| Phe (F) | Leu, Val, Ile, Ala, Tyr |
| Pro (P) | Ala |
| Ser S) | Thr |
| Thr (T) | Ser |
| Trp (W) | Phe, Tyr |
| Tyr (Y) | Trp, Phe, Thr, Ser |
| Val (V) | Ile, Leu, Met, Phe, Ala |

In some embodiments, another consideration for amino acid substitutions include whether or not the residue is located in the interior of a protein or is solvent exposed. For interior residues, conservative substitutions can include in some embodiments: Asp and Asn; Ser and Thr; Ser and Ala; Thr and Ala; Ala and Gly; Ile and Val; Val and Leu; Leu and Ile; Leu and Met; Phe and Tyr; Tyr and Trp. For solvent exposed residues, conservative substitutions can include in some embodiments: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, the Dayhoff matrix, the Grantham matrix, the McLachlan matrix, the Doolittle matrix, the Henikoff matrix, the Miyata matrix, the Fitch matrix, the Jones matrix, the Rao matrix, the Levin matrix, and the Risler matrix (summarized in, for example, Johnson & Overington (1993) J Mol Biol 233:716-738; see also the PROWL resource available at the website of The Rockefeller University, New York, N.Y., United States of America).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

In some embodiments, the presently disclosed subject matter provides an antibody or a nucleic acid encoding an antibody comprising the V_{H} and/or V_{L} of the scFV described above for the presently disclosed CAR. More particularly, in some embodiments, the presently disclosed subject matter provides a nucleic acid that encodes an antibody that binds to integrin αᵥβ₃, wherein the nucleic acid comprises SEQ ID NO: 3 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 3 (e.g., that encodes a V_{H}) and/or SEQ ID NO: 5 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 5 (e.g., that encodes a V_{L}). In some embodiments, the nucleic acid comprises both (i) SEQ ID NO: 3 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 3 and (ii) SEQ ID NO: 5 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 5.

In some embodiments, the nucleic acid further comprises a sequence encoding a linker for joining the V_{H} and V_{L} to one another. In some embodiments, the sequence comprises SEQ ID NO: 4 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto. In some embodiments, the nucleic acid comprises, consists essentially of, or consists of the sequence R₁-R₂-R₃, wherein: R₁ is SEQ ID NO: 3 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto; R₂ is SEQ ID NO: 4 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto; and R₃ is SEQ ID NO: 5 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto. In some embodiments, the nucleic acid sequence comprises, consists essentially of, or consists of R₁-R₂-R₃, wherein: R₁ is SEQ ID NO: 3; R₂ is SEQ ID NO: 4; and R₃ is SEQ ID NO: 5.

In some embodiments, the presently disclosed subject matter provides an antibody that binds to integrin αᵥβ₃, wherein the antibody comprises a V_{H} comprising SEQ ID NO: 13 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 13 and/or a light V_{L} comprising SEQ ID NO: 15 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 15. In some embodiments, the nucleic acid comprises both (i) SEQ ID NO: 13 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 13 and (ii) SEQ ID NO: 15 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to SEQ ID NO: 15.

In some embodiments, the antibody further comprises a linker for joining the V_{H} and V_{L} to one another. In some embodiments, the linker comprises or consists of glycine and serine residues or repeating subsequences comprising or consisting of glycine and serine residues. In some embodiments, the sequence comprises SEQ ID NO: 14 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto. In some embodiments, the nucleic acid comprises, consists essentially of, or consists of the sequence R₁-R₂-R₃, wherein: R₁ is SEQ ID NO: 13 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto; R₂ is SEQ ID NO: 14 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto; and R₃ is SEQ ID NO: 15 or a sequence at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical thereto. In some embodiments, the nucleic acid sequence comprises, consists essentially of, or consists of R₁-R₂-R₃, wherein: R₁ is SEQ ID NO: 13; R₂ is SEQ ID NO: 14; and R₃ is SEQ ID NO: 15.

In some embodiments, the antibody comprises a V_{H} comprising one, two, or three of the complementarity determining regions (CDRs), i.e., CDR-H1, CDR-H2, and CDR-H3, of the V_{H} of SEQ ID NO: 13, wherein CDR-H1 is amino acids 31-35 of SEQ ID NO: 13; CDR-H2 is amino acids 50-66 of SEQ ID NO: 13; and CDR-H3 is amino acids 99-106 of SEQ ID NO: 13. In some embodiments, the antibody comprises a V_{H} comprising amino acids 31-35 of SEQ ID NO: 13 as a first CDR, amino acids 50-66 of SEQ ID NO: 13 as a second CDR, and amino acids 99-106 of SEQ ID NO: 13 as a third CDR. In some embodiments, the antibody comprises a V_{L} comprising one, two, or three of the CDRs, i.e., CDR-L1, CDR-L2, and CDR-L3, of the V_{L} of SEQ ID NO: 15, wherein CDR-L1 is amino acids 24-34 of SEQ ID NO: 15; CDR-L2 is amino acids 50-56 of SEQ ID NO: 15, and CDR-L3 is amino acids 89-97 of SEQ ID NO: 15. In some embodiments, the antibody comprises a V_{L} comprising amino acids 24-34 of SEQ ID NO: 15 as a first CDR, amino acids 50-56 of SEQ ID NO: 15 as a second CDR, and amino acids 89-97 of SEQ ID NO: 15 as a third CDR. In some embodiments, the antibody comprises CDRs from both the V_{H} of SEQ ID NO: 13 and the V_{L} of SEQ ID NO: 15. In some embodiments, the antibody comprises all six CDRs (the three CDRs from the V_{H} of SEQ ID NO: 13 and the three CDRs from the V_{L} of SEQ ID NO: 15).

In some embodiments, the antibody further comprises one or more antibody constant region, such as a human antibody constant region. In some embodiments, the antibody is an antibody fragment. In some embodiments, the antibody is a scFV.

### III. Genetically Modified Cells and Methods of Making the Same

In some embodiments, the presently disclosed subject matter comprises a genetically modified cell that comprises a nucleic acid or vector of the presently disclosed subject matter or that expresses the presently disclosed CAR. In some embodiments, the presently disclosed subject matter provides a population of such cells (i.e., a composition comprising a plurality of such cells). In some embodiments, the cell is a cytolytic immune cell, such as a T cell or an NK cell. In some embodiments, the T cell is a NKT cell.

T cells or T lymphocytes are a type of lymphocyte that play a central role in cell-mediated immunity. They can be distinguished from other lymphocytes, such as B cells and NK cells, by the presence of a T-cell receptor (TCR) on the cell surface. There are various types of T cell. For example, helper T cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells can be either CD4+ or CD8+. Memory T cells typically express the cell surface protein CD45RO.

Regulatory T cells (Treg cells), formerly known as suppressor T cells, are involved in the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells. Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX. Adaptive Treg cells (also known as Tr1 cells or Th3 cells) can originate during a normal immune response.

Natural Killer cells (or NK cells) form part of the innate immune system. NK cells provide rapid responses to innate signals from virally infected cells in an MHC independent manner NK cells are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The genetically modified immune cells of the presently disclosed subject matter can be any of the cell types mentioned above or mixtures thereof. In some embodiments, the genetically modified immune cells comprise T cells.

The genetically modified immune cells of the presently disclosed subject matter can be created ex vivo either from a patient's own cells or from cells from a donor (e.g., a same species donor). Thus, the genetically modified immune cells can be autologous or allogenic with regard to a patient being treated with the genetically modified immune cells.

In some embodiments, the immune cells can be derived from ex vivo differentiation of inducible progenitor cells or embryonic progenitor cells to immune cells (e.g., the T cells or NK cells). Alternatively, an immortalized T-cell line which retains its lytic function and could act as a therapeutic can be used.

In some embodiments, the genetically modified immune cells are generated by introducing DNA or RNA coding for the CAR by one of many means including, but not limited to, transduction with a viral vector or transfection with DNA or RNA. In some embodiments, a population of immune cells can be obtained and activated and/or expanded prior to being transduced or otherwise genetically modified, for example, by treatment with an anti-CD3 monoclonal antibody. In some embodiments, the genetically modified cells (e.g., genetically modified T or NK cells) can be purified, for example, by selection on the basis of expression of the anti-integrin αᵥβ₃ antigen-binding domain.

More particularly, prior to expansion and genetic modification of the immune cells of the presently disclosed subject matter, a source of immune cells is obtained from a subject (i.e., either the subject to be treated with the genetically modified immune cells or another same species individual). Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments commercially available immune cell lines can be used. In some embodiments, immune cells can be obtained from a unit of blood collected from a subject using any number of techniques known to the skilled artisan. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis can be washed to remove the plasma fraction and to place the cells in an appropriate buffer or media for subsequent processing steps. In some embodiments, the cells are washed with phosphate buffered saline (PBS). In some embodiments, the wash solution lacks calcium and/or lacks magnesium and/or lacks all divalent cations. As those of ordinary skill in the art would readily appreciate a washing step can be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells can be resuspended in a variety of biocompatible buffers or other saline solution with or without buffer. Alternatively, the undesirable components of the apheresis sample can be removed and the cells directly resuspended in culture media.

In some embodiments, T cells are isolated from peripheral blood lymphocytes by lysing the red blood cells and depleting the monocytes, for example, by centrifugation through a gradient or by counterflow centrifugal elutriation. A particular subpopulation of T cells, such as CD3⁺, CD28⁺, CD4⁺, CD8⁺, CD45RA⁺, and CD45RO⁺ T cells, can be further isolated by positive or negative selection techniques. For example, in one embodiment, T cells are isolated by incubation with anti-CD3/anti-CD28-conjugated beads for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours. Longer incubation times can be used to isolate T cells in any situation where there are few T cells as compared to other cell types, such in isolating tumor infiltrating lymphocytes (TIC) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of the presently disclosed subject matter. In certain embodiments, it can be desirable to perform the selection procedure and use the “unselected” cells in the activation and expansion process. “Unselected” cells can also be subjected to further rounds of selection.

Enrichment of an immune cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In some embodiments, it can be desirable to enrich for or positively select for regulatory T cells which typically express CD4⁺, CD25⁺, CD62L^{hi}, GITR⁺, and FoxP3⁺. Alternatively, in some embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it can be desirable to significantly decrease the volume in which beads and cells are mixed together (i.e., increase the concentration of cells), to ensure maximum contact of cells and beads. For example, in some embodiments, a concentration of 2 billion cells/ml is used. In some embodiments, a concentration of 1 billion cells/ml is used. In some embodiments, greater than 100 million cells/ml is used. In some embodiments, a concentration of cells of 10, 15, 20, 25, 30, 35, 40, 45, or 50 million cells/ml is used. In some embodiments, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that weakly express target antigens of interest, such as CD28-negative T cells, or from samples where there are many tumor cells present (i.e., leukemic blood, tumor tissue, etc.). Such populations of cells can have therapeutic value and would be desirable to obtain. For example, using high concentration of cells allows more efficient selection of CD8⁺ T cells that normally have weaker CD28 expression.

In some embodiments, it can be desirable to use lower concentrations of cells. By significantly diluting the mixture of cells, surface interactions between particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells can be incubated on a rotator for varying lengths of time at varying speeds at either 2-10° C. or at room temperature.

Immune cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells can be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing can be used as well as uncontrolled freezing immediately at −20° C. or in liquid nitrogen.

In certain embodiments, cryopreserved cells are thawed and washed as described herein and allowed to rest for one hour at room temperature prior to activation using the methods of the presently disclosed subject matter.

Also provided in the context of the presently disclosed subject matter is the collection of blood samples or apheresis product from a subject at a time period prior to when the expanded cells as described herein are needed. As such, the source of the cells to be expanded can be collected at any time point necessary, and desired cells, such as T cells, isolated and frozen for later use in CAR T cell therapy for the treatment of cancer, as in the methods described herein. In some embodiments, a blood sample or an apheresis is taken from a generally healthy subject. In some embodiments, a blood sample or an apheresis is taken from a generally healthy subject who is at risk of developing a disease, but who has not yet developed a disease, and the cells of interest are isolated and frozen for later use. In certain embodiments, the T cells can be expanded, frozen, and used at a later time. In some embodiments, samples are collected from a patient shortly after diagnosis of a particular disease (e.g., GBM or another cancer) as described herein but prior to any treatments. In some embodiments, the cells are isolated from a blood sample or an apheresis from a subject prior to any number of relevant treatment modalities, including but not limited to chemotherapy, radiation, surgery, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. In some embodiments, the cells are isolated for a patient and frozen for later use in conjunction with (e.g., before, simultaneously, or following) chemotherapy, surgery (e.g., tumor resection) or radiation.

In some embodiments, T cells are obtained from a patient directly following a treatment for cancer. In this regard, it has been observed that following certain cancer treatments, in particular treatments with drugs that damage the immune system, shortly after treatment during the period when patients would normally be recovering from the treatment, the quality of T cells obtained can improved with regard to ability to expand ex vivo. Likewise, following ex vivo manipulation using the methods described herein, these cells can be in a preferred state for enhanced engraftment and in vivo expansion. Thus, it is contemplated within the context of the presently disclosed subject matter to collect blood cells, including T cells, NK cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy.

Prior to or after genetic modification of the immune cells (e.g., T cells) to express a CAR, the cells can be activated and expanded. Methods of activating and expanding T cells are described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005, each of which is incorporated herein by reference in its entirety.

Generally, T cells can be expanded by contact with a surface having attached thereto an agent that stimulates a CD3/TCR complex associated signal and a ligand that stimulates a co-stimulatory molecule on the surface of the T cells. In particular, T cell populations can be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, and XR-CD28 (Diaclone, Besancon, France).

In some embodiments, the primary stimulatory signal and the co-stimulatory signal for the T cell can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In some embodiments, the agent providing the co-stimulatory signal is bound to a cell surface and the agent providing the primary activation signal is in solution or coupled to a surface. In some embodiments, both agents can be in solution. In some embodiments, the agents can be in soluble form, and then cross-linked to a surface, such as a cell expressing Fc receptors or an antibody or other binding agent which will bind to the agents. In this regard, see for example, U.S. Patent Application Publication Nos. 2004/0101519 and 2006/0034810 (each of which is incorporated herein by reference in its entirety) for artificial antigen presenting cells (aAPCs) that are contemplated for use in activating and expanding T cells.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In some embodiments, a ratio of anti CD3:CD28 antibodies bound to the beads is used such that an increase in T cell expansion is observed as compared to the expansion observed using a ratio of 1:1. In some embodiments an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between.

Ratios of particles to cells from 1:500 to 500:1 and any integer values in between can be used to stimulate T cells or other target immune cells. As those of ordinary skill in the art can readily appreciate, the ratio of particles to cells can depend on particle size relative to the target cell. For example, small sized beads can only bind a few cells, while larger beads can bind many. In further embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). One of skill in the art will appreciate that a variety of other ratios can be suitable for use in the presently disclosed subject matter. In particular, ratios will vary depending on particle size and on cell size and type.

In some embodiments, the cells, such as T cells, are combined with agent-coated beads, the beads and the cells are subsequently separated, and then the cells are cultured. In some embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins can be ligated by allowing paramagnetic beads to which anti-CD3 and anti-CD28 are attached to contact the T cells. In one embodiment the cells (for example, 10⁴ to 10⁹T cells) and beads (for example, paramagnetic beads sold under the tradename DYNABEADS™ M-450 CD3/CD28 T (Life Technologies, Oslo, Norway) at a ratio of 1:1) are combined in a buffer, preferably PBS (without divalent cations such as, calcium and magnesium). Again, those of ordinary skill in the art can readily appreciate any cell concentration can be used. For example, the target cell can be very rare in the sample and comprise only 0.01% of the sample or the entire sample (i.e., 100%) can comprise the target cell of interest. Accordingly, any cell number is within the context of the presently disclosed subject matter. In some embodiments, it can be desirable to significantly decrease the volume in which particles and cells are mixed together (i.e., increase the concentration of cells), to ensure maximum contact of cells and particles. For example, in some embodiments, a concentration of about 2 billion cells/ml is used. In some embodiments, greater than 100 million cells/ml is used. In some embodiments, a concentration of cells of 10, 15, 20, 25, 30, 35, 40, 45, or 50 million cells/ml is used. In some embodiments, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that weakly express target antigens of interest, such as CD28-negative T cells. Such populations of cells can have therapeutic value and would be desirable to obtain in certain embodiments. For example, using high concentration of cells allows more efficient selection of CD8+ T cells that normally have weaker CD28 expression.

In some embodiments, the mixture can be cultured for several hours (about 3 hours) to about 14 days or any hourly integer value in between. In some embodiments, the mixture can be cultured for 21 days. In some embodiments, the beads and the immune cells are cultured together for about eight days. In some embodiments, the beads and immune cells (e.g., T cells) are cultured together for 2-3 days. Several cycles of stimulation can also be used such that culture time of immune cells can be 60 days or more. Conditions appropriate for T cell culture include an appropriate media (e.g., Minimal Essential Media or RPMI Media 1640 or, X-VIVO™ 15, (Lonza America, Inc., Morristown, N.J., United States of America)) that can contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, .alpha.-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be administered to a subject. The target cells are maintained under conditions necessary to support growth, for example, an appropriate temperature (e.g., 37° C.) and atmosphere (e.g., air plus 5% CO₂).

Immune cells that have been exposed to varied stimulation times can exhibit different characteristics. For example, typical blood or apheresed peripheral blood mononuclear cell products have a helper T cell population (T_{H}, CD4⁺) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Accordingly, in some embodiments, the presently disclosed subject matter provides a method of making an anti-αᵥβ₃ chimeric antigen receptor (CAR) immune cell, wherein the method comprises introducing into an immune cell a nucleic acid construct or vector as described hereinabove. In some embodiments, the nucleic acid construct or vector is introduced into the immune cell ex vivo. The construct or vector can be introduced via any suitable means as described hereinabove. In some embodiments, the vector is a virus vector (e.g., a retroviral vector or other virus vector) and the vector is introduced into the cell via transduction or transfection with the vector.

In some embodiments, the immune cell is a T cell, a NK cell or a NKT cell. In some embodiments, the T cells comprise central memory T cells; memory stem T cells, and/or naive T cells. In some embodiments, the method comprises obtaining a population of immune cells, such as by obtaining a biological sample comprising the immune cells, such as a blood serum sample. In some embodiments, the sample comprises PBMCs. In some embodiments, the method further comprises treating the sample prior to introduction of the nucleic acid construct or vector to obtain the desired immune cells. In some embodiments, the treating comprises activating and/or expanding the immune cells (e.g., via stimulation with anti-CD3/anti-CD28 magnetic beads).

### IV. Methods of Treating Cancer

In some embodiments, the presently disclosed subject matter provides a method of treating cancer in a subject in need thereof. In some embodiments, the cancer is selected from the group including, but not limited to, a CNS cancer (e.g., a brain cancer), a breast cancer, a cervical cancer, a bladder cancer, a lung cancer, a melanoma, a neuroblastoma, and a rhabdomyosarcoma (RMS). For example, in some embodiments, the lung cancer is a non-small cell lung (NSCL) cancer. In some embodiments, the breast cancer is a triple negative breast cancer optionally wherein said lung cancer is a non-small cell lung cancer or wherein said breast cancer is a triple negative breast cancer (i.e., a breast cancer that does not express and/or tests negative for estrogen receptors, progesterone receptors, and HER2 protein). In some embodiments, the cancer is a brain cancer. In some embodiments, the brain cancer is DIPG. In some embodiments, the brain cancer is GBM.

In some embodiments, the method comprises administering a therapeutically effective amount of a genetically modified immune cell as described herein (e.g., a T cell, a NK cell, or a NKT cell expressing a CAR comprising an anti-integrin αᵥβ₃ scFV, such as an scFV comprising SEQ ID NO: 13 and SEQ ID NO: 15 or amino acid sequences having at least about 70%, 75%, 80%, 85%, or about 90% identity thereto, or containing a nucleic acid, nucleic acid construct or vector encoding such a CAR, such as a nucleic acid comprising SEQ ID NO: 3 and SEQ ID NO: 5 or nucleic acid sequences having at least about 70%, 75%, 80%, 85%, or about 90% identity thereto). Once administered, the genetically modified cells can kill tumor cells in the subject. In some embodiments, the cells are able to replicate in vivo resulting in long-term persistence that can lead to sustained tumor control.

The CAR-modified immune cells of the presently disclosed subject matter can serve as a type of vaccine for ex vivo immunization and/or in vivo therapy in a mammal. In some embodiments, the method of treating cancer comprises: (a) obtaining a population of immune cells (e.g., human immune cells); (b) transducing at least a portion of the population of immune cells with a vector of as described herein (e.g., comprising a nucleic acid encoding an anti-αᵥβ₃ CAR of the presently disclosed subject matter) to provide a population of transduced human immune cells; and (c) administering the population of transduced human immune cells to the subject. In some embodiments, the population of immune cells is obtained from a blood serum sample. In some embodiments, the cells comprise PBMCs. In some embodiments, the obtained cells are treated to isolate a desired population of cells, such as central memory T cells, memory stem T cells, and naive T cells. In some embodiments, the treating comprises activation and/or expansion as described hereinabove.

In some embodiments, the subject is a human. In some embodiments, the subject is an adult human diagnosed with, being otherwise treated for, at risk for, or at risk of a recurrence of a cancer, such as a CNS cancer (e.g., a brain cancer), a breast cancer, a cervical cancer, a bladder cancer, a lung cancer, a melanoma, a neuroblastoma, and a RMS. In some embodiments, the cancer is a GBM. In some embodiments, the subject is a non-adult human (e.g., a human 18 years old or younger, such as 0-14 years old) diagnosed with, being otherwise treated for, at risk for, or at risk of a recurrence of a cancer selected from the group including, but not limited to, a CNS cancer (e.g. DIPG), melanoma, neuroblastoma, and a RMS. In some embodiments, the subject is a non-adult human diagnosed with, being otherwise treated for, or at risk of a recurrence of a DIPG. In some embodiments, the subject is human and the genetically modified immune cell can be autologous with respect to the subject. Alternatively, the genetically modified cells can be allogeneic, syngeneic or xenogeneic with respect to the subject.

The genetically modified cells can be administered either alone, or as a pharmaceutical composition in combination with diluents and/or with other components such as IL-2 or other cytokines or cell populations. Briefly, pharmaceutical compositions of can comprise a genetically modified cell or cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, compositions are formulated for intravenous administration.

Pharmaceutical compositions of the presently disclosed subject matter can be administered in a manner appropriate to the cancer to be treated (or prevented). The quantity and frequency of administration can be determined by such factors as the condition of the patient, and the type and severity of the cancer, although appropriate dosages can be determined by clinical trials.

When “a therapeutically effective amount”, the precise amount of the compositions of the presently disclosed subject matter to be administered can be determined by a physician with consideration of individual differences in age, weight, tumor size, extent of infection or metastasis, and condition of the patient (subject). It can generally be stated that a pharmaceutical composition comprising the immune cells described herein can be administered at a dosage of 10⁴ to 10⁹ cells/kg body weight, in some embodiments 10⁵ to 10⁶ cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments, it can be desired to administer activated T cells to a subject and then subsequently redraw blood (or have an apheresis performed), activate T cells therefrom according to the presently disclosed subject matter, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol can serve to select out certain populations of T cells.

The administration of the subject compositions can be carried out in any convenient manner, including by aerosol inhalation, injection, ingestion, transfusion, implantation or transplantation. The compositions described herein can be administered to a patient subcutaneously, intradermally, intratumorally, intranodally, intramedullary, intramuscularly, by intravenous (IV) injection, or intraperitoneally. In some embodiments, the genetically modified immune cell compositions are administered to a patient by intradermal or subcutaneous injection. In some embodiments, the genetically modified immune cell compositions can be administered systemically (e.g., by IV injection or infusion). In some embodiments, the compositions can be injected directly into a tumor, lymph node, or site of infection. In some embodiments, the genetically modified immune cells can be administered intracerebrally or intraperitonially.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Methods and Materials

### Human Tumor Cell Lines and Cell Culture

Tumor cell lines were obtained from the following sources: Patient-derived DIPG cell cultures were kindly provided by M. Monje (Stanford University), U87 from L. Lum (University of Virginia), and U251 were provided by D. Brautigan (University of Virginia). Maintenance and culture of DIPG lines was performed according to protocols established by the Monje lab. Briefly, DIPG lines were maintained in serum free medium (1:1 ratio of Neurobasal-A and D-MEM/F-12 (Invitrogen Corporation, Carlsbad, Calif., United States of America)) supplemented with B-27 without vitamin A (Invitrogen Corporation, Carlsbad, Calif., United States of America), epidermal growth factor (EGF), fibroblast growth factor (FGF), platelet-derived growth factor (PDGF)-AA, PDGF-BB (Shenandoah Biotechnology, Inc., Warwick, Pa., United States of America), Heparin (StemCell Technologies, Vancouver, Canada), HEPES, sodium pyruvate, non-essential amino acids (NEAA), L-Glutamine and Antibiotic-Antimycotic (all from Invitrogen Corporation, Carlsbad, Calif., United States of America). U87 and U251 were maintained in Dulbecco's Modified Eagle's medium (DMEM) containing 10% fetal bovine serum (FBS) and 1% penicillin/streptomycin. 293GP was maintained in DMEM with 10% FBS, 1 mM Sodium Pyruvate, 1% L-Glutamine and 1% penicillin/streptomycin. All other cell lines were maintained in Roswell Park Memorial Institute (RPMI) 1640 media or DMEM media each containing 10% FBS, 1% L-Glutamine and 1% penicillin/streptomycin. Human PBMCs were obtained from normal healthy donors by aphaeresis.

### CAR T Cell Construct Design, Synthesis, and Cloning

The scFv sequence for targeting integrin αᵥβ₃ was derived from the sequence for the monoclonal antibody clone LM609. The variable regions of the heavy and light chains were assembled with a linker comprising repeats of the sequence GGGGS (SEQ ID NO: 21). The scFv sequence was preceded by the human GM-CSF-R signal sequence and followed by human CD8α hinge and transmembrane domain. This was linked to either a CD28 or 4-1BB intracellular co-stimulatory signaling domain followed by the CD3zeta(ζ) signaling chain. The entire construct was generated by commercial gene synthesis. The αᵥβ₃ constructs were then cloned into a retroviral vector by blunt-ended restriction enzyme cloning. In short, the CD19.2K CAR domain was excised from the FMC.63 backbone vector and replaced with the αᵥβ₃ CAR domain. After sub-cloning, the correct sequences were confirmed by commercial DNA sequencing.

### Retrovirus Production and T Cell Transduction

Retroviruses encoding CAR T cell constructs were produced via transient transfection of 293GP cells. The day before transfection, 293GP cells were seeded on poly-D-lysine (10 mg/cm²) coated plates. 293GP cell lines were transfected in antibiotic free medium via Lipofectamine 2000 (Life Technologies, Carlsbad, Calif., United States of America) with plasmids encoding the CARs and RD114. Isolated human PBMCs were stimulated with human T cell activation and expansion beads (Life Technologies, Carlsbad, Calif., United States of America) at a 1:1 ratio with 40 IU/ml IL-2 for 3 days. Retroviral supernatants were added to plates coated with Retronectin (Takara Bio USA, Mountain View, Calif., United States of America) and centrifuged for 2 hours at 2,000×g. Activated T cells were transduced on day 3 and 4 and cultured in AIM-V medium (Thermo Fisher Scientific, Waltham, Mass., United States of America) containing 5% FBS, 1% L-Glutamine, 1% HEPES and 1% Pen/Strep Antibiotic with 300 IU/ml IL-2. Beads were removed on day 5 and T cells were expanded in medium with 300 IU/ml IL-2. T cells were expanded again on day 7.

### Flow Cytometry

Cell surface expression of integrin αᵥβ₃ on DIPG and GBM tumor cells was evaluated by staining with PE-conjugated anti-integrin αᵥβ₃ antibody (LM609; Millipore Sigma, Burlington, Mass., United States of America) or isotype control antibody. Human T cell analysis of ex vivo expanded CAR T cells, and T cells isolated from mouse blood, brain, and spleen was performed using antibodies against CD3 (HIT3a), CD4 (OKT4), CD8 (SK1), CD45RO (UCH1.1), CD45RA (H100), CCR7 (G043H7), CD127 (A019D5), PD-1 (EH12.2H7), LAG-3 (11C3C65), TIM-3 (F38-2E2), CD45 (2D1), and TCF-1 (7F11A10) (from BioLegend, San Diego, Calif., United States of America). Expression of integrin αᵥβ₃ CAR was detected using biotinylated protein L (Thermo Fisher Scientific, Waltham, Mass., United States of America) followed by streptavidin-conjugated PE (BioLegend, San Diego, Calif., United States of America). Intracellular staining for TCF-1 was performed using Foxp3/Transcription Factor Staining Buffer Set (eBioscience Inc., San Diego, Calif., United States of America). All samples were stained with dye sold under the tradename ZOMBIE AQUA™ Fixable Viability dye (BioLegend, San Diego, Calif., United States of America). Sample data was acquired on a NovoCyte 3005 Flow Cytometer (ACEA Biosciences Inc., San Diego, Calif., United States of America) and analyzed using data analysis software sold under the tradename FLOWJO™ v10 (FlowJo LLC, Ashland, Oreg., United States of America).

### Cytotoxicity and Cytokine Production Assays

Cell death of DIPG tumor cell lines and GBM were evaluated using a luciferase-based assay and bioluminescence was measured on an IVIS Spectrum instrument (PerkinElmer Inc., Waltham, Mass., United States of America). Initially, tumor cells were lentivirally transduced with eGFP and firefly luciferase (eGFP/ffLuc). Tumor cells (5,000-10,000 per well) were co-incubated with control non-transduced T cells, CD19.28ζ CAR, αᵥβ₃.28ζ CAR, or αᵥβ₃.BBζ CAR T cells for 18 hours at effector-to-target (E:T) ratios ranging from 10:1 to 2:5:1. Tumor cell lysis was determined by measuring residual luciferase activity and calculated as follows: percent lysis=100−(((average signal from T cell treated tumor wells)/(average signal from untreated target tumor wells))×100). In vitro cytokine production by CAR T cells was evaluated through co-incubation of control non-transduced T cells, CD19.28ζ CAR, αᵥβ₃.28ζCAR, or αᵥβ₃.BBζ CAR T cells with tumor cell targets at a 1:1 ratio (500,000 cells each), and effector T cell numbers were normalized across each group with transduction efficiency of CAR⁺ T cells. Supernatants of the co-cultures were collected after 24 hours and were tested for IFN-γ, IL-2, and TNF-a by ELISA (BioLegend, San Diego, Calif., United States of America).

For the triple-negative breast, RMS, melanoma, cervical squamous cell carcinoma, NSCL, and bladder tumor cells, in vitro anti-tumor cytotoxicity was evaluated by measuring electrical impedance of adherent tumor cells (xCelligence; ACEA Biosciences, Inc., San Diego, Calif., United States of America).

### Orthotopic Xenogeneic Mouse Models and CAR T Cell Treatments

For DIPG, 5×10⁵ patient-derived SU-DIPG-36 cells expressing firefly luciferase were implanted by stereotactic injection into the pons of 6-8-week-old NOD scid IL2Rg⁻ (i.e., NOD scid gamma (NSG)) mice (stereotactic coordinates from lambda were as follows: M/L, +1 mm; A/P, −0.8 mm; D/V, −5 mm). For GBM, 5×10⁵ U87 cells expressing firefly luciferase were implanted by stereotactic injection into the pons of NSG mice (stereotactic coordinates from bregma were as follows: M/L, +3 mm; A/P, 0.7 mm; D/V, −3 mm). On Day 21 post-engraftment, 2×10⁶ integrin αᵥβ₃ CAR T cells were delivered intratumorally. Tumor burden was assessed at least once a week via bioluminescent imaging following intraperitoneal (IP) injection of D-luciferin (Caliper IVIS Spectrum; PerkinElmer Inc., Waltham, Mass., United States of America).

### Immunohistochemistry and Digital Histology

A multiple organ normal tissue microarray (TMA) containing 20 organ types (BN1002b) was purchased from US BioMax, Inc (Derwood, Md., United States of America). The TMA was stained with anti-integrin αᵥβ₃ antibody (23C6; R&D Systems), developed using DAB chromogen, and counter-stained with hematoxylin. An Olympus BX-41 microscope (Olympus Corporation, Tokyo, Japan) with attached DP71 camera was used for digital scanning of slides at 20× objective. The scanned TMA was then evaluated for integrin αᵥβ₃ staining in QuPath, an open-source software application for digital histopathology analysis (Bankhead et al., Scientific Reports, 2017, 7:16878). Briefly, the normal tissue TMA was de-arrayed for evaluation of individual tissue cores. Automated positive cell detection for each core was then performed at three threshold levels and H-scores were calculated.

### Data and Statistical Analysis

All statistical analysis was performed with GraphPad Prism (GraphPad Software, San Diego, Calif., United States of America). Flow cytometry data was analyzed using software sold under the tradename FLOWJO™ v10 (FlowJo LLC, Ashland, Oreg., United States of America). IVIS bioluminescence images and data were analyzed with Living Image Software (PerkinElmer Inc., Waltham, Mass., United States of America).

### Example 1

### Tumor Cell Expression of Alphaᵥ Beta₃ and Minimal Integrin αᵥβ₃ Expression in Normal Human Tissues

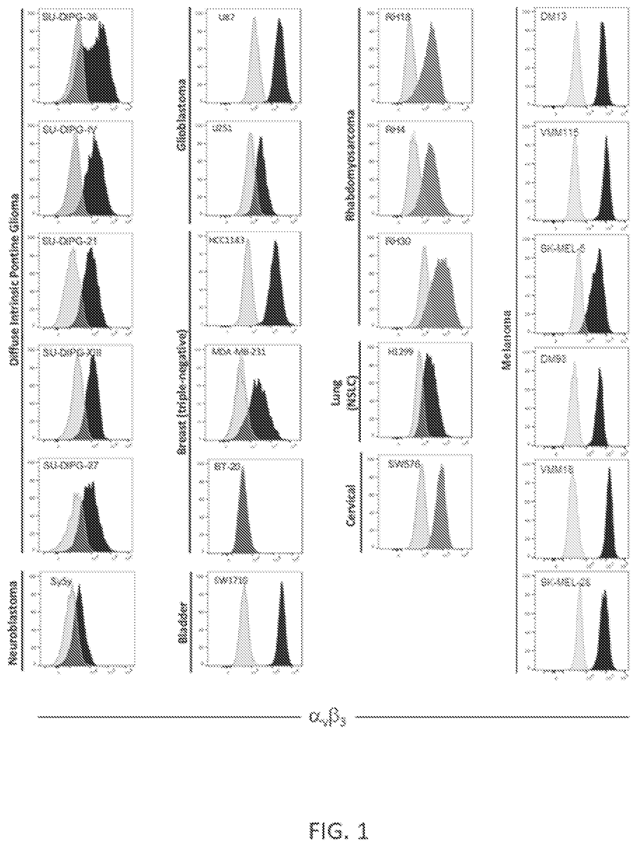

Tumor cell line expression of integrin αᵥβ₃ was measured by flow cytometry. More particularly, cell surface expression of integrin αᵥβ₃ was evaluated by flow cytometry staining with phycoerythrin-conjugated anti-integrin αᵥβ₃ monoclonal antibody (clone LM609) on diffuse intrinsic pontine glioma (DIPG), neuroblastoma, glioblastoma (GBM), triple-negative breast, bladder, rhabdomyosarcoma (RMS), lung, cervical squamous cell carcinoma, and melanoma tumor cell lines. See FIG. 1. Integrin αᵥβ₃ expression was detectable on the majority of tumor lines that were screened. For example, for the CNS tumors DIPG and GBM, integrin αᵥβ₃ was highly expressed on DIPG lines (SU-DIPG-36, SU-DIPG-IV, SU-DIPG-XIII) and exhibited moderate expression on the majority of other DIPG cells that were screened. Integrin αᵥβ₃ was also highly expressed on GBM (U87 and U251). The mean fluorescence intensity (MFI) ratio (MFI/MFI isotype control) for integrin αᵥβ₃ in several GBM and DIPG cell lines was calculated and ranked from highest to lowest, as shown in Table 3, below.

TABLE 3
MFI ratios of GBM and DIPG tumor cell lines.

| Cell Line | MFI ratio |
|---|---|
| GBM U87 | 17 |
| SU-DIPG-36 | 16 |
| SU-DIPG-IV | 8 |
| SU-DIPG-21 | 6 |
| SU-DIPG-XIII | 5 |
| SU-DIPG-17 | 5 |
| SU-DIPG-27 | 4 |
| GBM U251 | 2 |

### Example 2

### Design and Phenotypic Characterization of CAR T Cells Re-Directed Against Integrin Alphaᵥ Beta₃

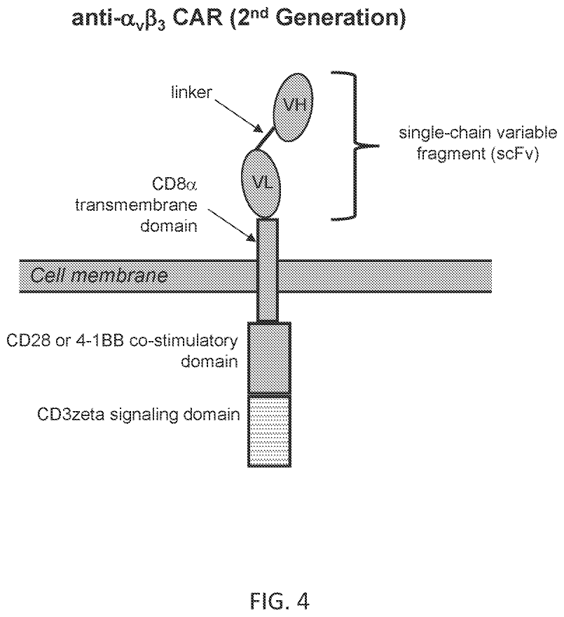

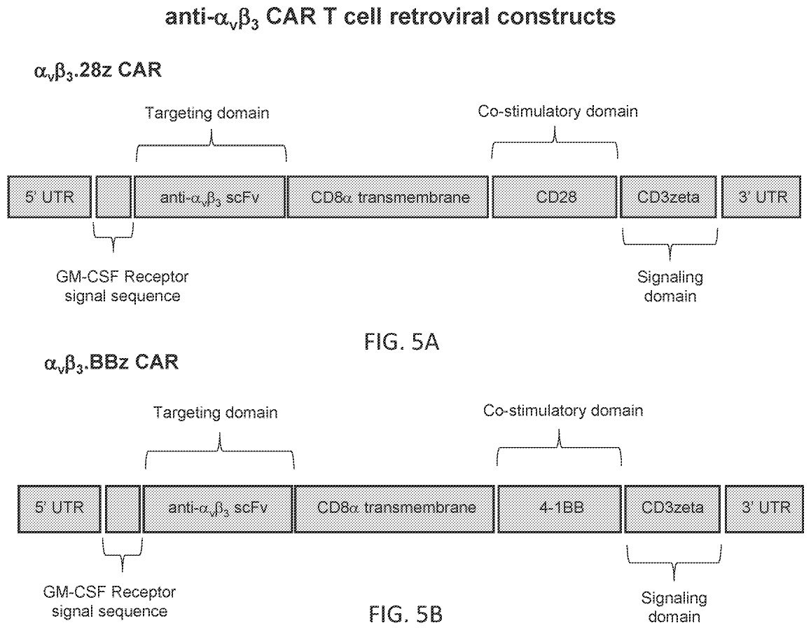

FIG. 4 shows a schematic diagram of an integrin αᵥβ₃ CAR construct of the presently disclosed subject matter. In the extracellular domain is a single chain variable fragment (scFV) comprising the variable regions of the heavy (V_{H}) and light (V_{L}) chains linked by a short linker peptide, and in the intracellular domain is a CD3-zeta (CD3ζ) signaling domain of the T-cell receptor (TCR) and the co-stimulatory domain of CD28 or 4-1BB. The design of the CAR construct against integrin αᵥβ₃ was based on the monoclonal antibody clone LM609 for the scFv domain. LM609 has been previously evaluated in various clinical studies and found to be safe, but with no reported treatment efficacy. Retroviral constructs for two exemplary retroviral vectors are shown in FIGS. 5A and 5B. More particularly, an anti-αᵥβ₃.28z CAR T cell construct is shown in FIG. 5A and an anti-αᵥβ₃.BBz CAR T cell construct is shown in FIG. 5B. The CAR constructs were sub-cloned into a retroviral vector using blunt-ended cloning.

More particularly, the nucleic acid and amino acid sequences of the αᵥβ₃.28z CAR constructs were as follows:

αᵥβ3.28z CAR DNA and Amino Acid sequences
Human GM-CSF Receptor signal peptide:
(SEQ ID NO: 2)
ATGCTTCTCCTGGTGACAAGCCTTCTGCTCTGTGAGTTACCACACCCAG
CATTCCTCCTGATCCCA
(SEQ ID NO: 12)
MLLLVTSLLLCELPHPAFLLIP
Alpha v beta3 single-chain variable fragment
heavy chain:
(SEQ ID NO: 3)
CAGGTGCAGCTGGTGGAGTCTGGGGGAGGCGTTGTGCAGCCTGGAAGGT
CCCTGAGACTCTCCTGTGCAGCCTCTGGATTCACCTTCAGTAGCTATGA
CATGTCTTGGGTTCGCCAGGCTCCGGGCAAGGGTCTGGAGTGGGTCGCA
AAAGTTAGTAGTGGTGGTGGTAGCACCTACTATTTAGACACTGTGCAGG
GCCGATTCACCATCTCCAGAGACAATAGTAAGAACACCCTATACCTGCA
AATGAACTCTCTGAGAGCCGAGGACACAGCCGTGTATTACTGTGCAAGA
CATAACTACGGCAGTTTTGCTTACTGGGGCCAAGGGACTACAGTGACTG
TTTCTAGT
(SEQ ID NO: 13)
QVQLVESGGGVVQPGRSLRLSCAASGFTFSSYDMSWVRQAPGKGLEWVA
KVSSGGGSTYYLDTVQGRFTISRDNSKNTLYLQMNSLRAEDTAVYYCAR
HNYGSFAYWGQGTTVTVSS
Linker:
(SEQ ID NO: 4)
GGAGGCGGCGGAAGCGGAGGCGGCGGAAGCGGAGGCGGCGGAAGCGGAG
GCGGCGGAAGC
(SEQ ID NO: 14)
GGGGSGGGGSGGGGSGGGGS
Alpha v beta 3 single-chain variable fragment
light chain:
(SEQ ID NO: 5)
GAGATTGTGCTAACTCAGTCTCCAGCCACCCTGTCTCTCAGCCCAGGAG
AAAGGGCGACTCTTTCCTGCCAGGCCAGCCAAAGTATTAGCAACCACCT
ACACTGGTATCAACAAAGGCCTGGTCAAGCCCCAAGGCTTCTCATCAAG
TATCGTTCCCAGTCCATCTCTGGGATCCCCGCCAGGTTCAGTGGCAGTG
GATCAGGGACAGATTTCACCCTCACTATCTCCAGTCTGGAGCCTGAAGA
TTTTGCAGTCTATTACTGTCAACAGAGTGGCAGCTGGCCTCACACGTTC
GGAGGGGGGACCAAGGTGGAAATTAAG
(SEQ ID NO: 15)
EIVLTQSPATLSLSPGERATLSCQASQSISNHLHWYQQRPGQAPRLLIK
YRSQSISGIPARFSGSGSGTDFTLTISSLEPEDFAVYYCQQSGSWPHTF
GGGTKVEIK
HindIII restriction enzyme site:
(SEQ ID NO: 6)
GCATCAAGCTTGTCG
(SEQ ID NO: 16)
ASSLS
CD8 alpha transmembrane domain:
(SEQ ID NO: 7)
TTCGTGCCGGTCTTCCTGCCAGCGAAGCCCACCACGACGCCAGCGCCGC
GACCACCAACACCGGCGCCCACCATCGCGTCGCAGCCCCTGTCCCTGCG
CCCAGAGGCGTGCCGGCCAGCGGCGGGGGGCGCAGTGCACACGAGGGGG
CTGGACTTCGCCTGTGATATCTACATCTGGGCGCCCTTGGCCGGGACTT
GTGGGGTCCTTCTCCTGTCACTGGTTATCACCCTTTACTGCAACCACAG
GAAC
(SEQ ID NO: 17)
FVPVFLPAKPTTTPAPRPPTPAPTIASQPLSLRPEACRPAAGGAVHTRG
LDFACDIYIWAPLAGTCGVLLLSLVITLYCNHRN
CD28 cytoplasmic co-stimulatory signaling domain:
(SEQ ID NO: 8)
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC
(SEQ ID NO: 18)
RSKRSRLLHSDYMNMTPRRPGPTRKHYQPYAPPRDFAAYRS
CD3 zeta signaling domain:
(SEQ ID NO: 10)
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA
(SEQ ID NO: 20)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In the αᵥβ₃.BBz construct, the co-stimulatory domain nucleic acid and amino acid sequences were:

4-1BB cytoplasmic co-stimulatory signaling domain:
(SEQ ID NO: 9)
CGTTTCTCTGTTGTTAAACGGGGCAGAAAGAAGCTCCTGTATATATTCA
AACAACCATTTATGAGACCAGTACAAACTACTCAAGAGGAAGATGGCTG
TAGCTGCCGATTTCCAGAAGAAGAAGAAGGAGGATGTGAACTG
(SEQ ID NO: 19)
RFSVVKRGRKKLLYIFKQPFMRPVQTTQEEDGCSCRFPEEEEGGCEL

Additional Nucleic Acids Used:

PmlI restriction enzyme site:
(SEQ ID NO: 1)
TGACCACGTGGCACC
HpaI restriction enzyme site:
(SEQ ID NO: 11)
TGACGTTAACGCACC

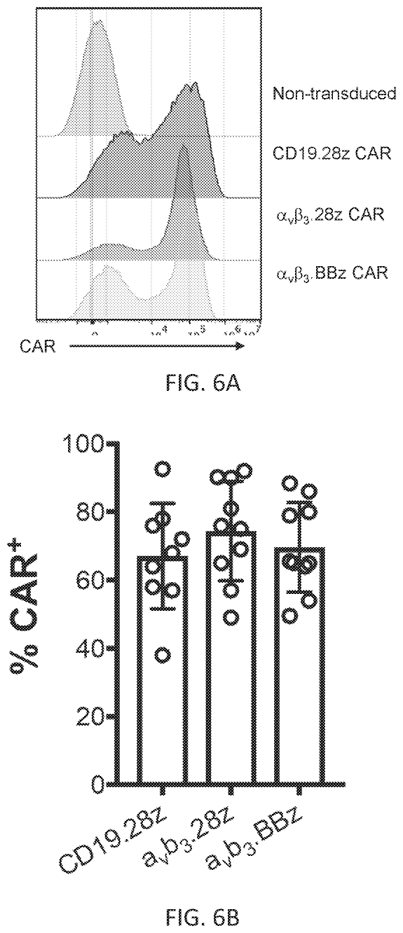

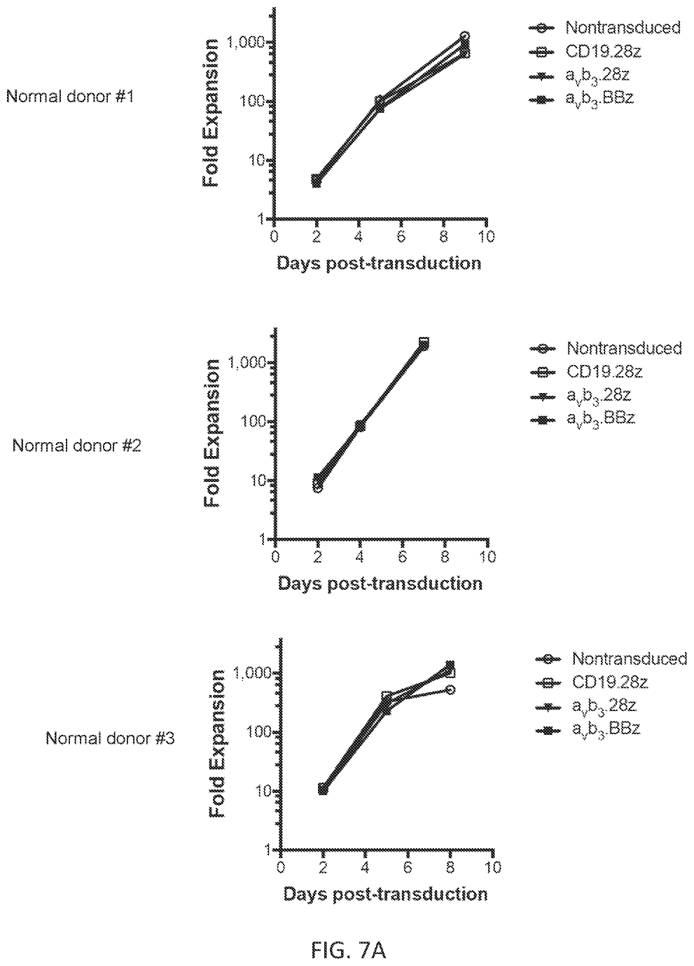

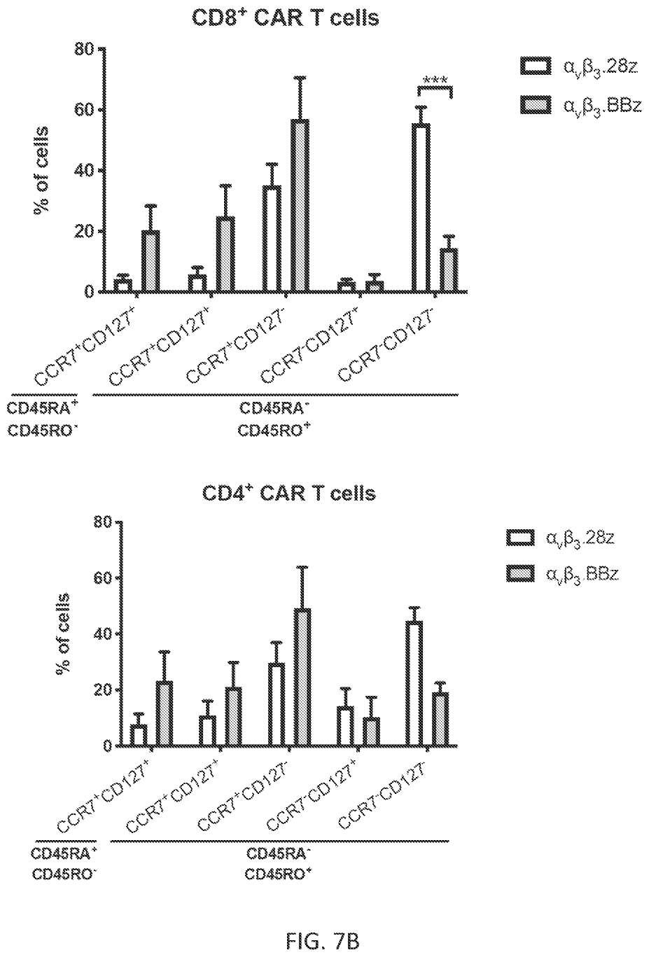

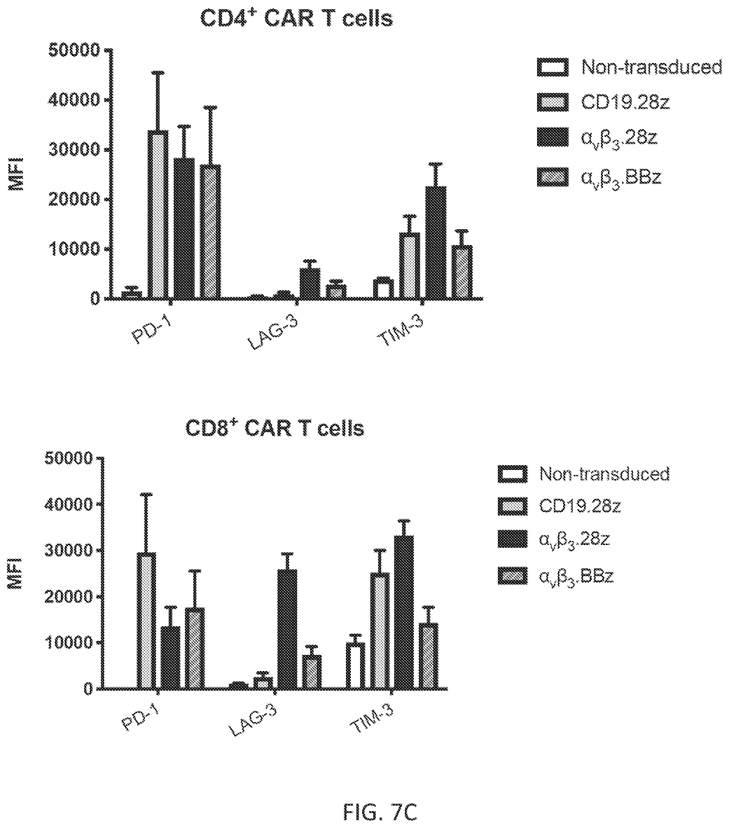

To prepare the CAR T cells, peripheral blood mononuclear cells (PBMCs) were stimulated for 3 days with anti-CD3/anti-CD28-coated magnetic beads in the presence of human IL-2 (40 μl/ml). Stimulated PBMCs were then transduced via retronectin-bound retrovirus containing one of the two integrin αᵥβ₃ CAR constructs or a control construct (CD19.28z CAR) on two consecutive days. Following expansion, CAR surface expression was measured using flow cytometry by incubating cells with biotinylated-L protein followed by staining with fluorochrome-conjugated streptavidin and anti-CD3 antibody. See FIG. 6A. The αᵥβ₃.28ζ and αᵥβ₃.BBζ CARs were highly expressed by transduced T cells, exhibiting an average of 76% and 66% positive, respectively, across multiple normal donors. See FIG. 6B. FIG. 6B shows that the transduced cells had a high percentage of CAR⁺ T cells in three normal donors across multiple experiments. CAR positivity and surface expression levels (e.g. MFI) of αᵥβ₃.28ζ and αᵥβ₃.BBζ CARs were comparable to the CD19.28ζ CAR, which was included throughout this study as a negative control and for comparison purposes. In addition, robust expansion of CAR T cells was observed following retroviral expansion in the three normal donors. Integrin αᵥβ₃ CAR T cells exhibited vigorous ex vivo expansion ranging from approximately 100- to 1,000-fold by day 7-9 post-transduction. See FIG. 7A. The expanded CAR T cells exhibited a comparable distribution ratio of CD4 and CD8 T cell subsets.

Less-differentiated CAR T cells and memory populations have been shown to exhibit superior anti-tumor responses in some pre-clinical studies. Therefore, the memory cell populations of integrin αᵥβ₃ CAR T cells following ex vivo expansion were assessed. Evaluation of CAR T cell phenotype by flow cytometry revealed a spectrum of memory and effector subset differentiation. Expanded αᵥβ₃.28ζ and αᵥβ₃.BBζ CAR T cells were comprised of effector and central memory subsets as well as stem cell memory T (TSCM)-like cells. There were no differences in the overall proportions of CD45RO⁺ and CD45RA⁺ populations between αᵥβ₃.28ζ and αᵥβ₃.BBζ CAR T cells. Both CD8 and CD4 T cell populations of integrin αᵥβ₃ CAR T cells harboring the 4-1BB co-stimulatory domain showed a trend towards a less-differentiated state compared to CAR T cells containing CD28, but there were no statistically significant differences between the two among most subsets, except in the frequency of CD8⁺CCR7⁻CD127⁻ effector memory cells. See FIG. 7B. Expression profiles for the inhibitory receptors PD-1, LAG-3, and TIM-3 on αᵥβ₃.28ζ and αᵥβ₃.BBζ CAR T cells following ex vivo expansion were analyzed. Collectively, there were no major differences in inhibitory receptor expression in integrin αᵥβ₃ CARs compared to non-transduced and CD19 CAR T cells. LAG-3 and TIM-3 tended to be lower in 4-1BB containing CARs, but these observations were not statistically significant. There was no difference in PD-1 expression. See FIG. 7C. Thus, αᵥβ₃.28ζ and αᵥβ₃.BBζ CAR T cells exhibited low levels of inhibitory receptors following ex vivo production.

### Example 3

### Integrin αᵥβ₃ CAR T Cells Exhibit Anti-Tumor Activity Against DIPG and GBM

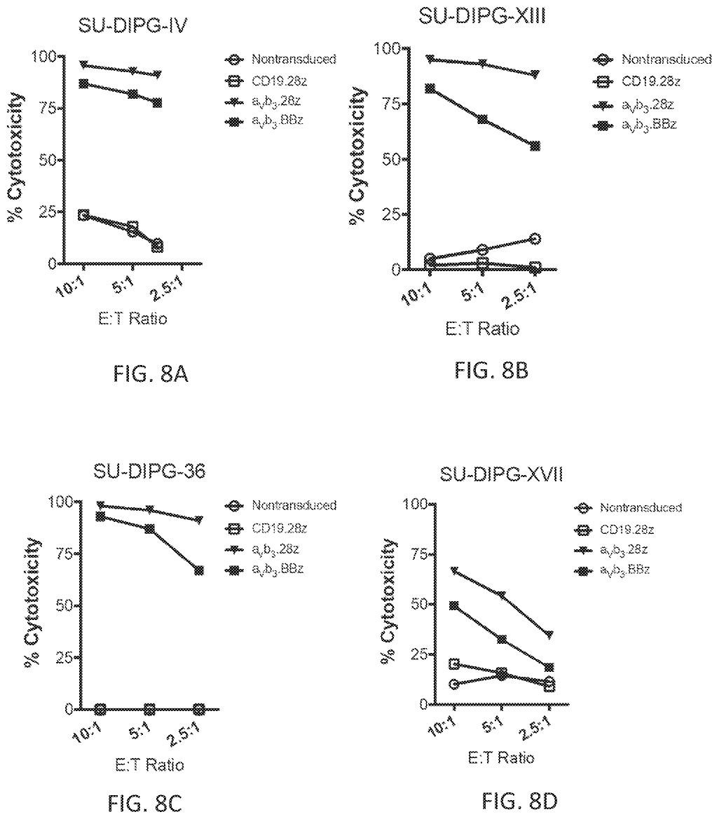

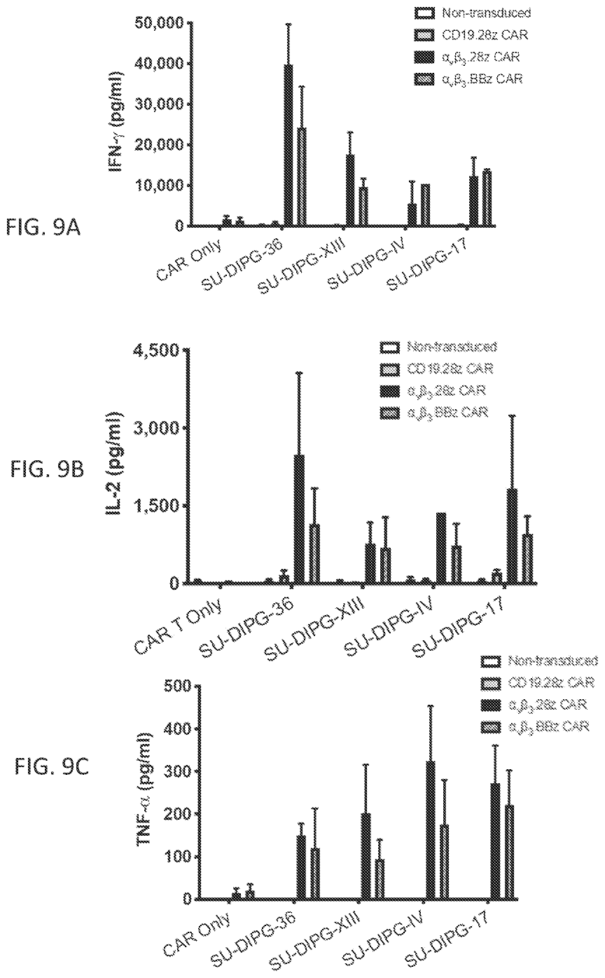

The anti-tumor activity of integrin αᵥβ₃ CAR T cells was evaluated by determining cytotoxicity and effector cytokine production against DIPG tumor cells in vitro. CAR T cell-mediated cytotoxicity was evaluated against DIPG lines, SU-DIPG-36, SU-DIPG-XIII, SU-DIPG-IV, and SU-DIPG-17 by exposing DIPG cells to non-transduced T cells, CD19, αᵥβ₃.28ζ, or αᵥβ₃.BBζ CAR T cells at E:T ratios ranging from 10:1 to 2.5:1 and measured using a bioluminescence-based culture assay to monitor tumor cell survival. Both integrin αᵥβ₃ CAR T cells exhibited substantial tumor cell killing against each DIPG line at all E:T ratios, with αᵥβ₃.BBζ CAR T cells showing a modest decrease, yet still high level of killing, at the lowest E:T ratio. See FIGS. 8A-8D. Little to no cytotoxicity of DIPG cell lines was observed when co-cultured with NT and CD19 CAR T cells. DIPG tumor cells also induced significant levels of effector cytokine production by integrin αᵥβ₃ CAR T cells, but not controls, as determined by measurements of secreted IFN-γ, IL-2, and TNF-α. See FIGS. 9A-9C. These data show the specific and robust anti-tumor responses of integrin αᵥβ₃ CAR T cells against DIPG tumor cells.

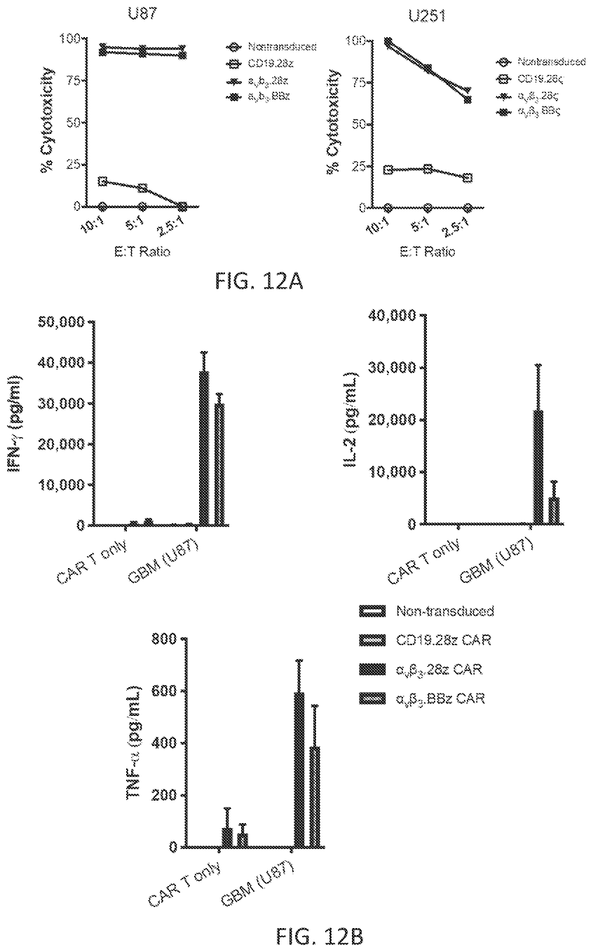

The efficacy of integrin αᵥβ₃ CAR T cells against GBM tumor cells in vitro was also evaluated. Anti-tumor activity of integrin αᵥβ₃ CAR T cells was evaluated against the GBM cell lines U87 and U251. Both αᵥβ₃.28ζ and αᵥβ₃.BBζ CAR T cells exhibited a high degree of tumor cell killing at all E:T ratios. See FIG. 12A. Further, integrin αᵥβ₃ CAR T cells produced effector cytokines upon tumor cell exposure. See FIG. 12B.

These data demonstrated that DIPG and GBM brain tumor cells expressing integrin αᵥβ₃ are highly susceptible to αᵥβ₃.28ζ and αᵥβ₃.BBζ CAR T cell anti-tumor responses.

### Example 4

### Integrin αᵥβ₃ CAR T Cells Exhibit Rapid Tumor Regression of Well-Established Orthotopic DIPG Tumors

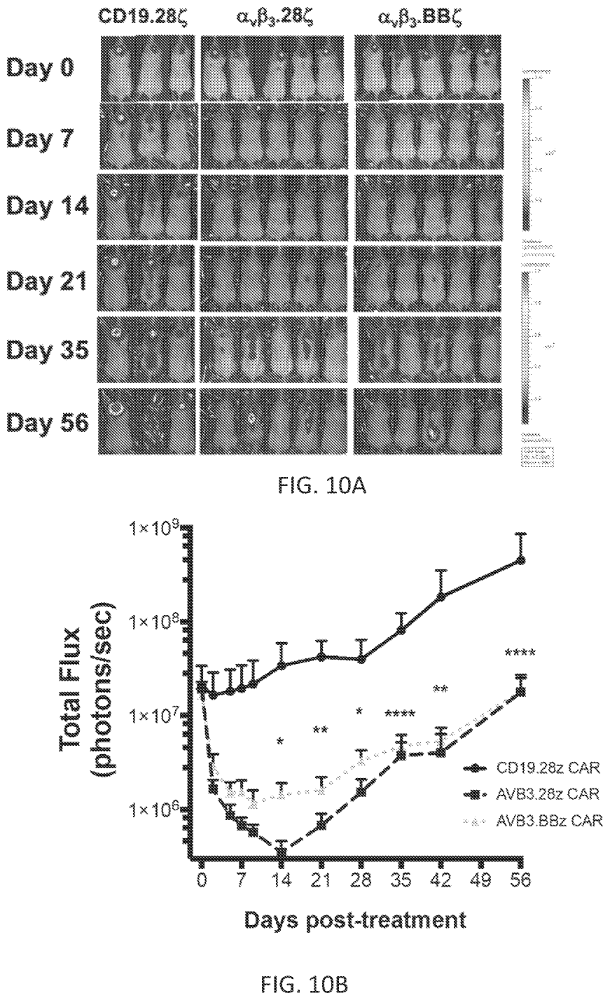

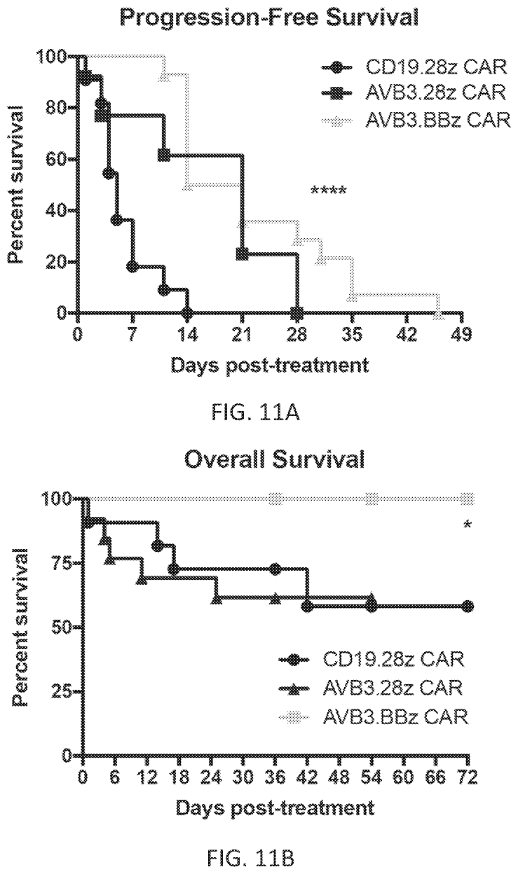

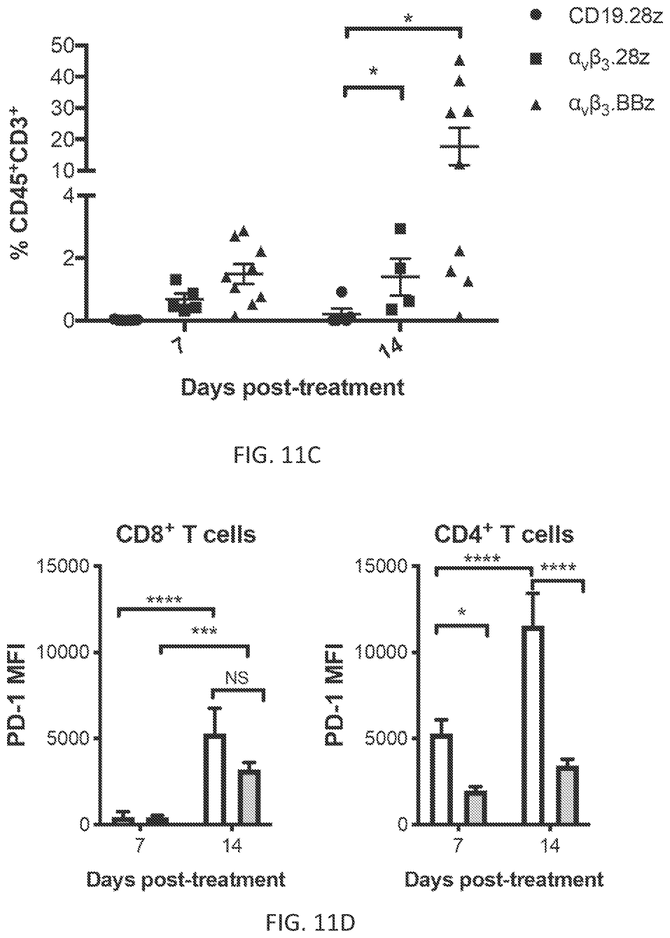

In order to validate the anti-tumor activity of integrin αᵥβ₃ CAR T cells and to demonstrate in vivo efficacy, an orthotopic mouse xenograft model for DIPG was utilized. First, NOD scid gamma (NSG) mice were implanted with SU-DIPG-36 cells expressing firefly luciferase into the pons region of the brainstem using previously established stereotactic coordinates. See Mount et al. (2018); Marigil et al. (2017), and Venkatesh et al. (2017). Tumor cell engraftment within the pons was confirmed by H&E staining. At three weeks post-implant, pre-treatment tumor burden was assessed using bioluminescent imaging (BLI). At this time, DIPG tumors were large and well-established. Tumor-bearing mice were then administered 2×10⁶ control CD19, αᵥβ₃.28ζ, or αᵥβ₃.BBζ CAR T cells by intratumoral (IT) injection. In three independent experiments, using CAR cells generated from different donors, rapid tumor regression was observed within the first week of treatment in mice administered integrin αᵥβ₃ CAR T cells. See FIGS. 10A and 10B. In contrast, tumors in mice receiving CD19 CAR T cells continued to progressively grow. By day 14 post-treatment, tumors in αᵥβ₃.28ζ CAR T cell treated mice were reduced to background bioluminescent levels. Similarly, tumors in mice treated with αᵥβ₃.BBζ CAR T cells were also significantly reduced, but remained detectable. Tumors did eventually recur in all mice, however, longitudinal monitoring showed that integrin αᵥβ₃ CAR T cells slowed or partially inhibited DIPG growth for an extended period of time as evidenced by the length of progression-free survival. See FIG. 11A. Of note, tumor recurrence in CAR-treated mice was slow over time and remained at or below initial pre-treatment levels for the duration of the experiment (8 weeks post-treatment). Mice surviving long-term required euthanasia due to the development of graft versus host disease, so additional survival and tumor monitoring could not be completed. Across three separate cohorts, disease resulted in lethality in 42% of mice given CD19 CAR T cells, whereas 100% of mice that received αᵥβ₃.BBζ CAR T cells survived the duration of the experiment. See FIG. 11B. Unexpectedly, 39% of mice that were treated with αᵥβ₃.28ζ CAR T cells died during the course of the experiment, of which most occurred within the first 11 days after injection, potentially due to treatment-induced toxicity. These results demonstrated that integrin αᵥβ₃ CAR T cells can act rapidly to significantly reduce DIPG tumors in mice, prolong tumor growth upon relapse, and extend survival.

The reduction in tumor burden coincided with a significant expansion of αᵥβ₃.28ζ and αᵥβ₃.BBζ CAR T cells within the circulation compared to CD19 control CAR T cells. See FIG. 11C. To assess the impact of antigen exposure on integrin αᵥβ₃ CAR T cells in vivo, inhibitory receptor expression was analyzed on CAR T cells in the blood on day 7 and 14 post-treatment. PD-1 levels on CD8⁺ CAR T cells increased from day 7 post-treatment to day 14 post-treatment in both αᵥβ₃.28ζ and αᵥβ₃.BBζ groups. See FIG. 11D. Only PD-1 levels on CD4⁺ αᵥβ₃.28ζ CAR T cells increased from day 7 post-treatment to day 14 post-treatment. There was no statistically significant increase in LAG-3 or TIM-3 expression levels from day 7 to day 14 post-treatment on either CAR T cell treatment group. In general, αᵥβ₃.BBζ CAR T cells exhibited significantly lower levels of inhibitory receptor expression compared to αᵥβ₃.28ζ CAR T cells. In conclusion, integrin αᵥβ₃ CAR T cells responding to DIPG tumors in vivo do not appear to show signs of exhaustion during the first 14 days of treatment, as there was not an overt nor wide-spread increase in inhibitory receptors, a characteristic indication of the development of T cell exhaustion. It remains possible that integrin αᵥβ₃ CAR T cells can have become exhausted at later timepoints. Therefore, CAR T cell exhaustion cannot be definitively ruled out as a mechanism leading to DIPG tumor recurrence in the present orthotopic xenograft model.

### Example 5

### Integrin αᵥβ₃ CAR T Cells Eradicate Established Glioblastoma Tumors in Mice

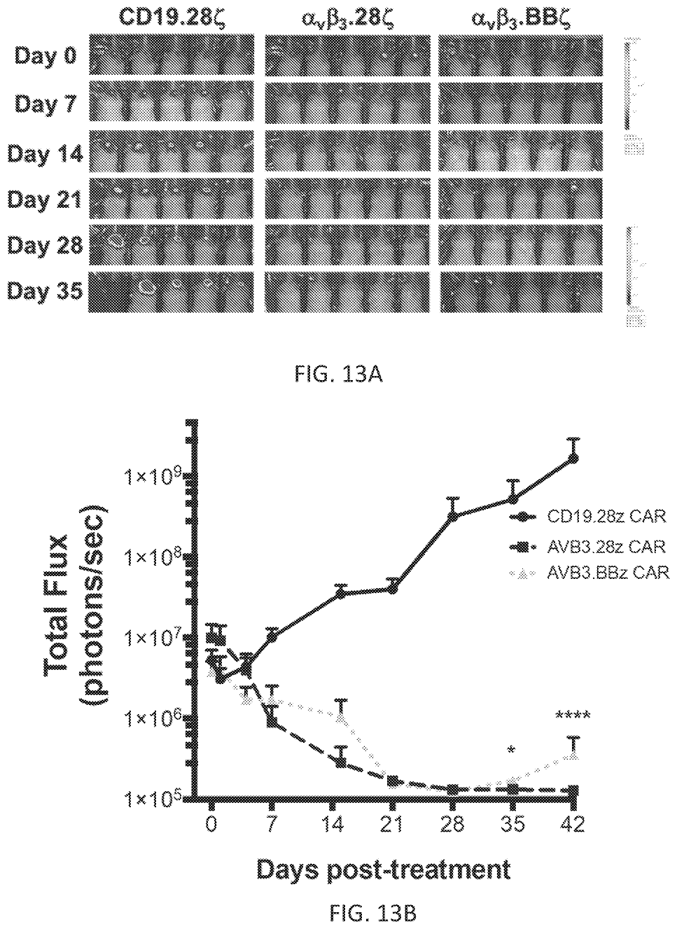

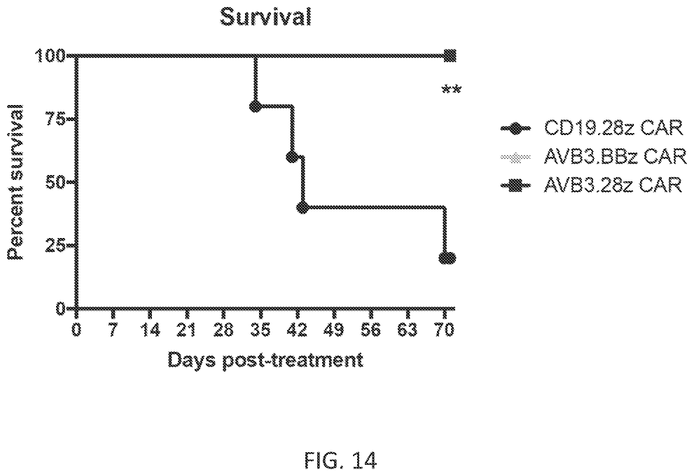

To test the in vivo anti-tumor activity of integrin αᵥβ₃ CAR T cells against GBM, U87 tumor cells were orthotopically implanted into the right forebrain of NSG mice via stereotactic injection. Tumors were allowed to grow for three weeks. GBM tumor-bearing mice were administered 2×10⁶ control CD19, αᵥβ₃.28ζ or αᵥβ₃.BBζ CAR T cells by IT injection. Reduction in tumor burden was evident by day 7 post-treatment, and progressively declined until it was completely eliminated by day 21. See FIGS. 13A and 13B. Meanwhile, GBM tumors in control CD19 CAR T cell-treated mice continued to grow aggressively during this time period. In contrast to treatment of DIPG tumors, IT treatment of GBM with αᵥβ₃.28ζ CAR T cells did not result in any deaths from treatment related toxicity. Without being bound to any one theory, this suggests that toxicity from CAR T cell treatment can manifest differently in the pons compared to other regions of the brain. Mice treated with integrin αᵥβ₃ CAR T cells were completely protected from lethality as 100% of mice survived long-term, whereas, 80% of control mice died by day 70 post-treatment. See FIG. 14. Of note, all integrin αᵥβ₃ CAR T cell treated mice remained tumor-free for the duration of the experiment. These data showed that integrin αᵥβ₃ CAR T cell treatment can be effective at completely eliminating GBM tumors in vivo without any observable evidence of toxicity, nor tumor recurrence.

### Example 6

### Systemic Administration of Integrin αᵥβ₃ CAR T Cells Leads to Complete Regression of Glioblastoma (GBM)

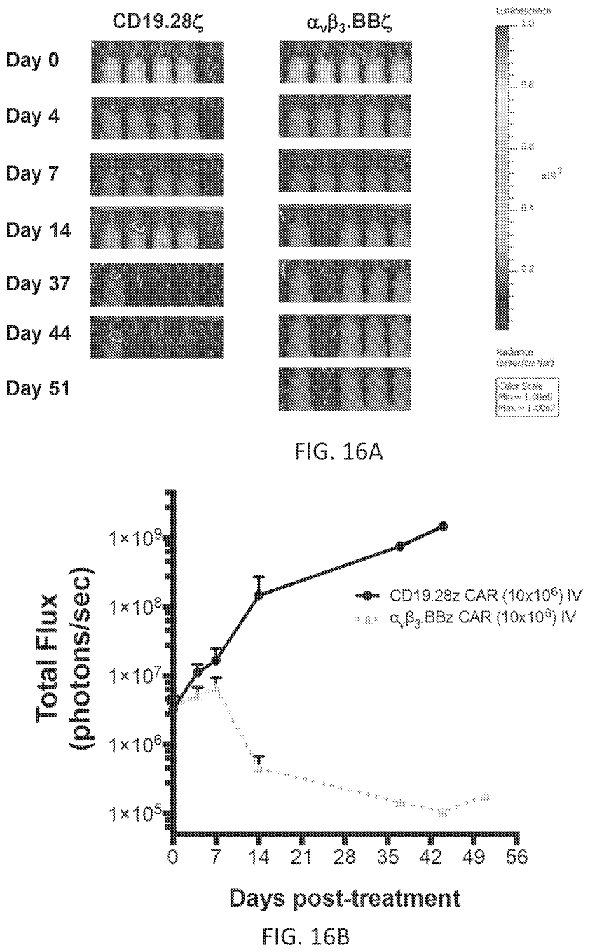

The results described in the examples above demonstrated effective anti-tumor activity of integrin αᵥβ₃ CAR T cells injected intratumorally. To determine if CAR T cells administered in a systemic manner via intravenous injection could migrate to orthotopic GBM tumors, mice were injected intravenously with 10×10⁶ CD19.28ζ or αᵥβ₃.BBζ CAR T cells. Tumor burden was monitored by bioluminescent imaging. See FIG. 16A. By day 14 post-treatment, complete tumor regression was evident in mice treated with αᵥβ₃.BBζ CAR T cells. See FIGS. 16A and 16B. These results demonstrated that integrin integrin αᵥβ₃ CAR T cells can be administered systemically to effectively treat GBM tumors.

### Example 7

### Integrin αᵥβ₃ CAR T Cells Develop into Long-Lived Memory Cells Following Successful GBM Treatment

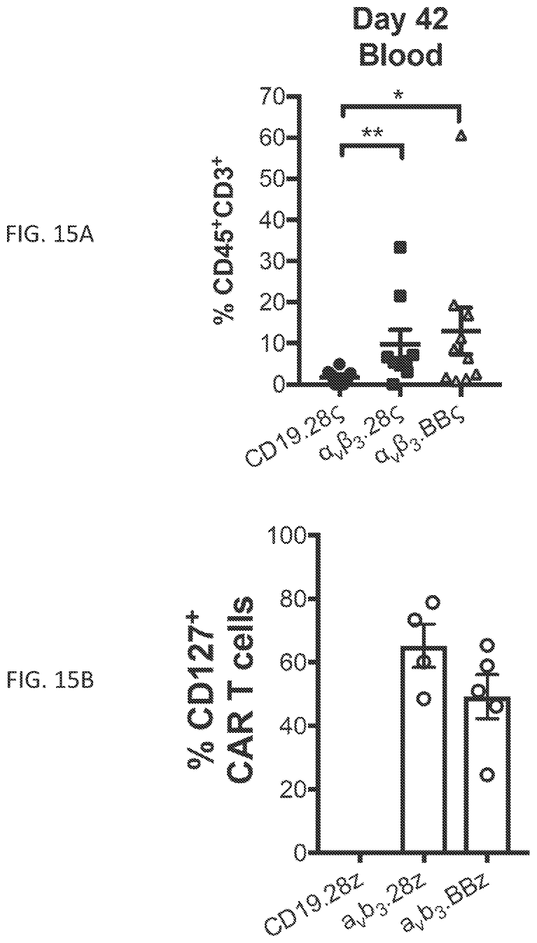

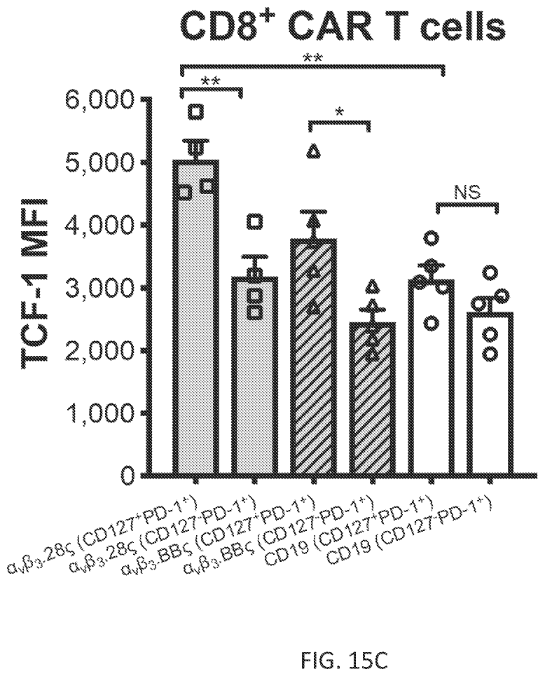

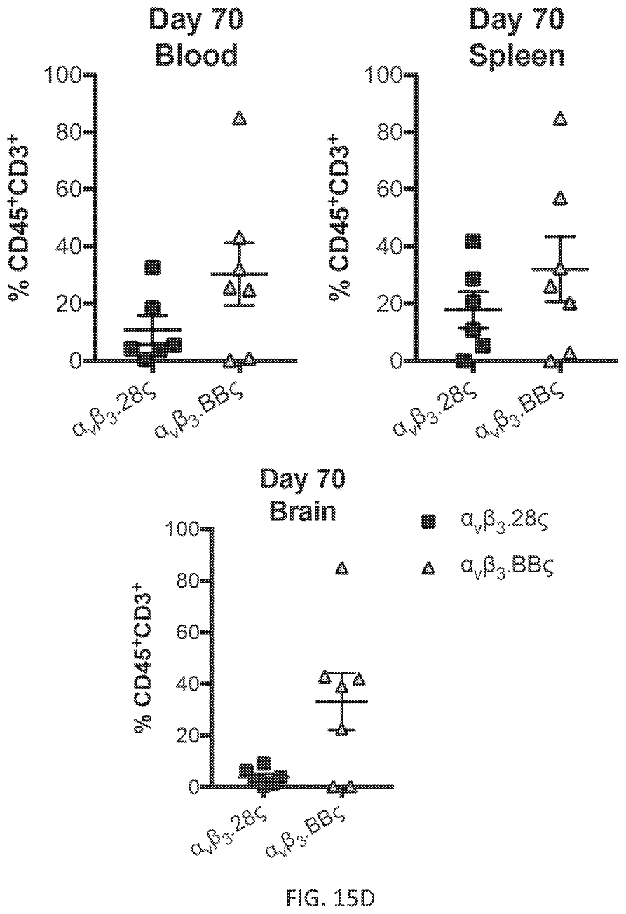

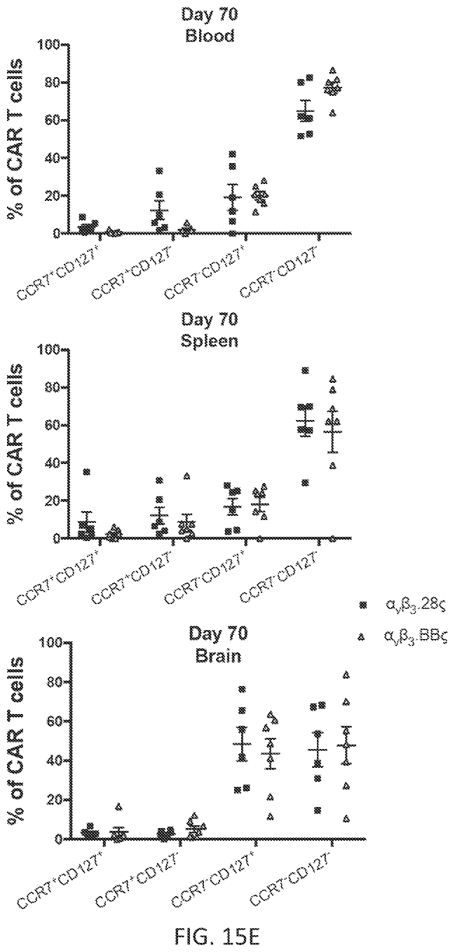

Studies were conducted to determine whether long-lived, memory CAR T cells develop following eradication of GBM tumors. Both αᵥβ₃.28ζ and αᵥβ₃.BBζ CAR T cells demonstrated exceptional persistence following tumor clearance as human T cells were easily detectable in blood at day 42 post-treatment. See FIG. 15A. A large proportion of αᵥβ₃.28ζ and αᵥβ₃.BBζ CAR T cells expressed the memory marker CD127 (see FIG. 15B), but were also PD-1⁺. Interestingly, mice treated with αᵥβ₃.28ζ CAR T cells had a significantly higher percentage of CD127⁺ cells compared to mice treated with αᵥβ₃.BBζ CARs. Some antigen-experienced T cells that exhibit properties of memory and PD-1 expression, yet retain the ability to self-renew in the absence of antigen, are responsive to checkpoint inhibition, and express the transcription factor TCF-1. See Kratchmarov (2018), Kallies et al. (2020), Zhang et al. (2020), and Wieland et al. (2017). TCF-1 regulates the stem-like functions of pre-exhausted T cells (PD-1^{Int}), and TCF-1⁺ tumor-infiltrating lymphocytes were shown to be elevated in melanoma patients who had responded to checkpoint receptor inhibition. See Siddiqui et al. (2019). Thus, a study was carried out to determine if integrin αᵥβ₃ CAR T cells expressed elevated levels of TCF-1 following successful treatment of GBM tumors. It was observed that the CD8⁺CD127⁺PD-1⁺ populations in αᵥβ₃.28ζ and αᵥβ₃.BBζ CAR-treated mice expressed significantly higher levels of TCF-1 relative to the more terminally differentiated CD127⁻PD-1⁺ populations. See FIG. 15C. As expected, TCF-1 expression was not increased in CD8⁺CD127⁺PD-1⁺ cells in mice treated with CD19 CAR T cells. CD4⁺CD127⁺PD-1⁺CAR T cells also exhibited elevated TCF-1 expression, but it was slightly lower than in CD8⁺ T cells. These data demonstrated that antigen-experienced integrin αᵥβ₃ CAR T cells capable of self-renewal can develop following eradication of GBM tumors.

Lastly, persistence of integrin αᵥβ₃ CAR T cells was evaluated 70 days following treatment of GBM tumors. It was observed that αᵥβ₃ CAR T cells were still present at day 70 post-treatment in blood, spleen, and brain. See FIG. 15D. Analysis of memory cell subsets by staining for cell-surface memory markers CD127 and CCR7 on CAR T cells was performed. The spectrum of memory T cell differentiation appeared similar in the blood, spleen, and brain. Although the highest proportion of CAR cells were the more differentiated CCR7⁻CD127⁻ subset, substantial frequencies of CCR7⁺CD127⁺, CCR7⁺CD127⁻, and CCR7⁻CD127⁺ subsets were detected in all tissues in which T cells were recovered. See FIG. 15E. The results of these experiments showed that integrin αᵥβ₃ CAR T cells develop memory and persist long-term in vivo.

### Example 8

### Normal Human Tissue Exhibits Minimal Integrin αᵥβ₃ Expression

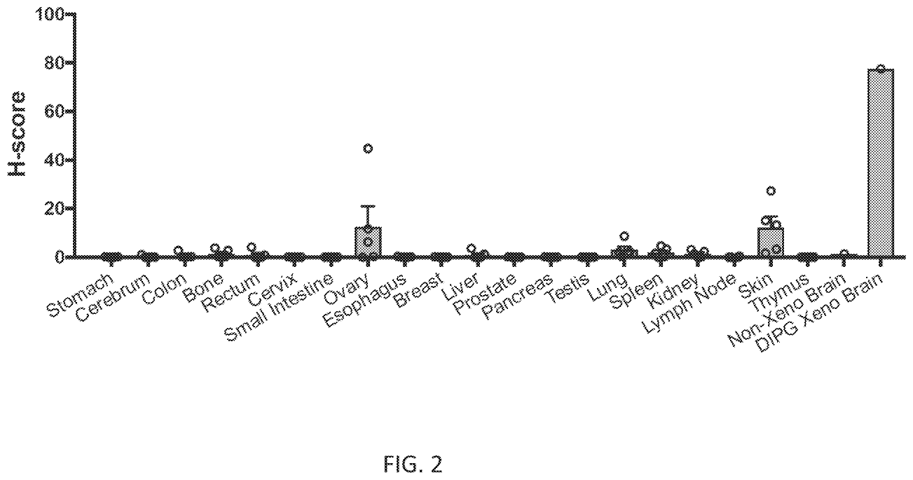

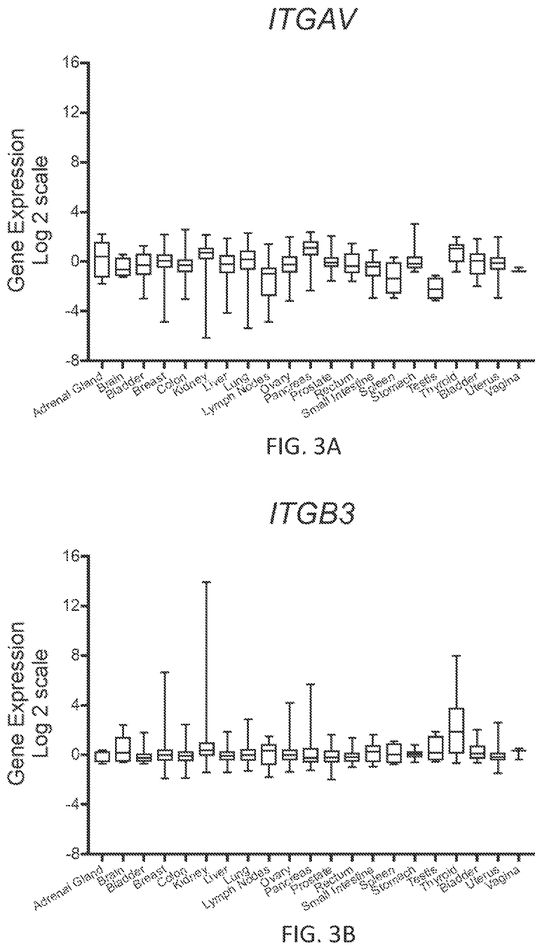

Given the high-level of CAR T cell activity in patients following antigen exposure, limiting “on-target, off-tumor” toxicities due to normal tissue expression of antigen is imperative for safe clinical usage. While integrin αᵥβ₃ expression has been shown to be primarily restricted to endothelial cells in newly forming blood vessels and absent in resting endothelial cells in normal organ systems, additional screening for its presence in normal tissues was performed. First, the expO Normal Tissue Expression array was accessed via the National Cancer Institute Oncogenomics Database in order to perform gene expression analysis of ITGAV and ITGB3. Neither ITGAV nor ITGB3 mRNA were highly expressed in the represented normal organ tissues. See FIGS. 3A and 3B. Secondly, the expression of integrin αᵥβ₃ was assessed by immunohistochemistry in a normal human TMA consisting of 20 organ types, each from five individual donors. Integrin αᵥβ₃ staining was absent in nearly all organ tissue cores stained or exhibited only minimal staining in others as determined by H-score. See FIG. 2. Of note, skin (3 of 5 donors) and ovary (2 of 4 donors), exhibited detectable but modest staining. These results provided further validation of the limited expression of integrin αᵥβ₃ in normal healthy tissues, supporting the targeting of this integrin by CAR T cells.

### Example 9

### Discussion of Example 1-8

CAR T cells targeting integrin αᵥβ₃ represents an attractive candidate for successful therapeutic treatment of malignant and fatal brain tumors. According to the presently disclosed subject matter, it was determined that integrin αᵥβ₃ expression on human DIPG and GBM tumor cells can be targeted by CAR T cells. More particularly, integrin αᵥβ₃ CAR T cells, containing either CD28 or 4-1BB co-stimulatory domains, mediate strong anti-tumor responses in the form of robust cytotoxicity and effector cytokine production against multiple patient-derived DIPG tumor lines and GBM cell lines. Importantly, integrin αᵥβ₃ CAR T cells were highly effective in vivo, mediating significant regression of orthotopic brain tumors and displaying long-lived persistence and memory development. Given the dire prognoses of high-grade gliomas like DIPG and GBM and the absence of effective therapies to treat them, integrin αᵥβ₃ CAR T cell treatment offer a promising avenue for these patients.

DIPG tumors are notoriously infiltrative. See Qin et al. (2017), Hashizume et al. (2012); and Kluiver et al (2020). In the orthotopic DIPG studies carried out according to the presently disclosed subject matter, a DIPG line that exhibits aggressive growth features (in vitro and in vivo observations) was utilized, which likely resulted in spreading to other areas of the brain, and almost certainly contributes to the challenge of achieving successful treatment. According to the present studies, tumors were allowed to grow in mice for three weeks prior to administering CAR T cell treatment. Without being bound to any one theory, during this time, invasion of other areas and niches of the brain by infiltrating DIPG cells likely occurred as has been demonstrated to happen in human disease and in DIPG mouse xenografts. See Qin et al. (2017) and Hashizume et al. (2012). Despite the aggressive nature of DIPG, tumor burden was significantly lowered, and progression-free survival and overall survival was extended through treatment with integrin αᵥβ₃ CAR T cells. Of note is the possible toxicity driven by integrin αᵥβ₃ CAR T cells that utilize CD28 co-stimulation in some mice with DIPG tumors. CAR T cell-induced toxicity was observed in a subset of mice with DIPG following treatment with GD2 CARs containing 4-1BB co-stimulation. See Mount et al. (2018). This study, along with the present results, suggests that the anatomical location of DIPG in the pons is a factor, given the critical functions performed in this area of the CNS. It is also interesting to speculate on the role of co-stimulatory domains (CD28 vs. 4-1BB) in CAR signaling and how these impact toxicities. In the case of orthotopic GBM tumors, complete clearance by integrin αᵥβ₃ CAR T cells was observed without any ensuing tumor recurrence. Additionally, no signs of CAR-related toxicity in GBM mice was observed following intra-tumoral delivery. These observations suggest potential differences in the development of treatment-related toxicity based on the anatomical location of gliomas.

An unresolved area of interest in the use of CAR T cells targeting integrin αᵥβ₃, is their potential capacity to recognize and respond to cells of the tumor vasculature expressing integrin αᵥβ₃. The anti-integrin αᵥβ₃ scFv in the presently disclosed CAR does not bind to murine integrin αᵥβ₃. Thus, determining the contribution of tumor-associated endothelial cells being target by CAR T cells is not possible based on the present studies in mice. In a study using echistatin as the CAR targeting domain against αᵥβ₃, destruction of tumor blood vessels in C57BL/6 mice implanted with B16 melanoma cells was observed, but not resting blood vessels within normal tissues (see Fu et al., 2013) and significantly stalled tumor growth.

In summary, the presently disclosed subject matter demonstrates that CAR T cells can be used to target integrin αᵥβ₃-expressing gliomas and that they exhibit strong anti-tumor responses in both in vitro cytotoxicity assays and in vivo using orthotopic xenograft models for human DIPG and GBM. The presently disclosed subject matter demonstrates the ability to targeting integrins, particularly integrin αᵥβ₃, for anti-tumor activity. The results demonstrate robust responses in tumor-bearing mice, coupled with the restricted nature of integrin αᵥβ₃ expression in normal tissues, suggesting both highly effective and safe use of integrin αᵥβ₃ CAR T cells for the treatment of DIPG and GBM in patients.

### Example 10

### Integrin αᵥβ₃ CAR T Cells Exhibit In Vitro and In Vivo Activity Against Non-CNS Tumors

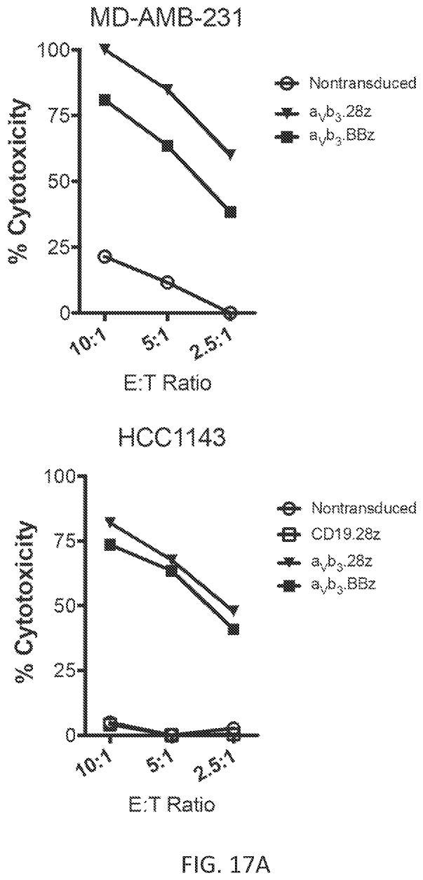

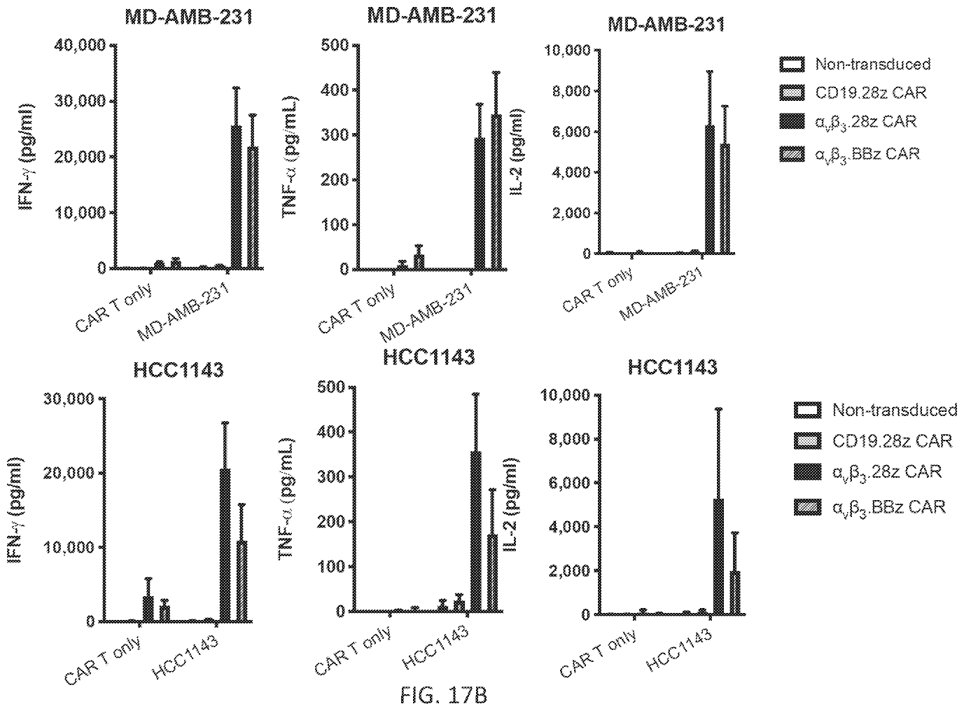

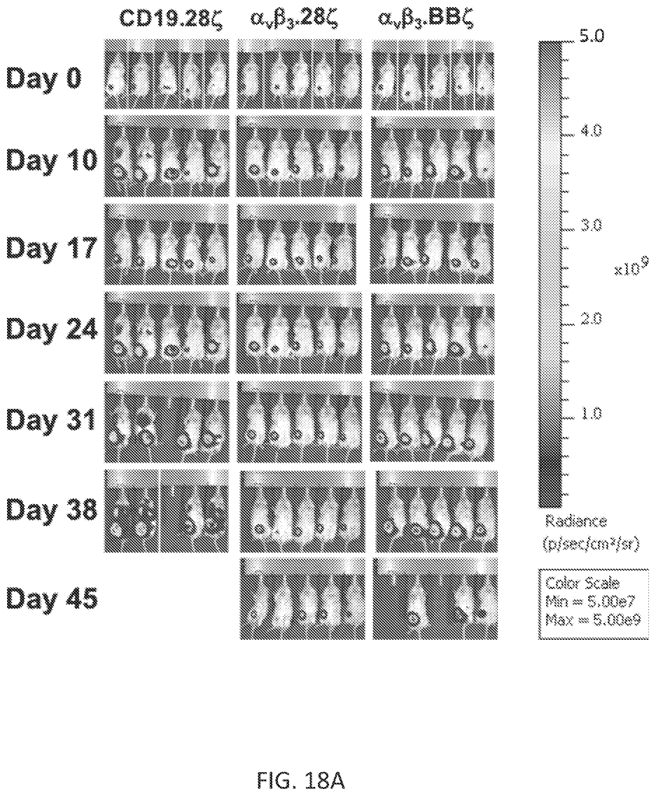

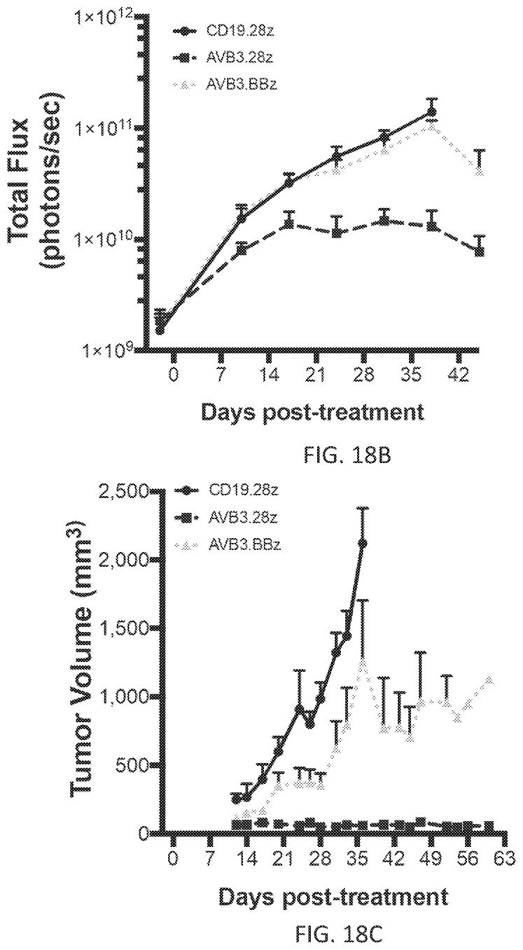

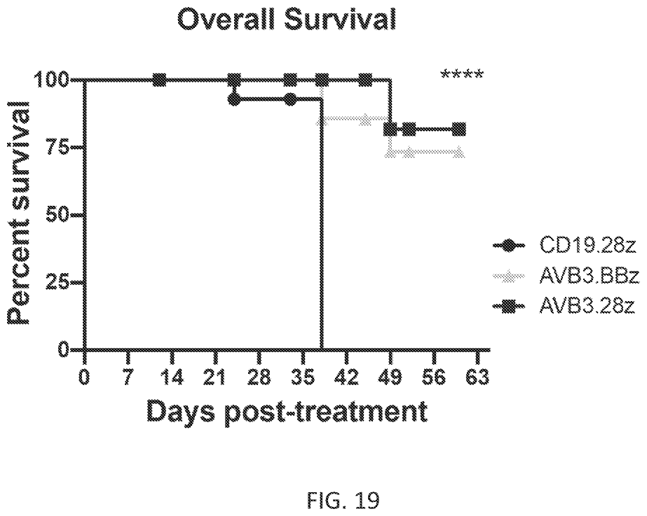

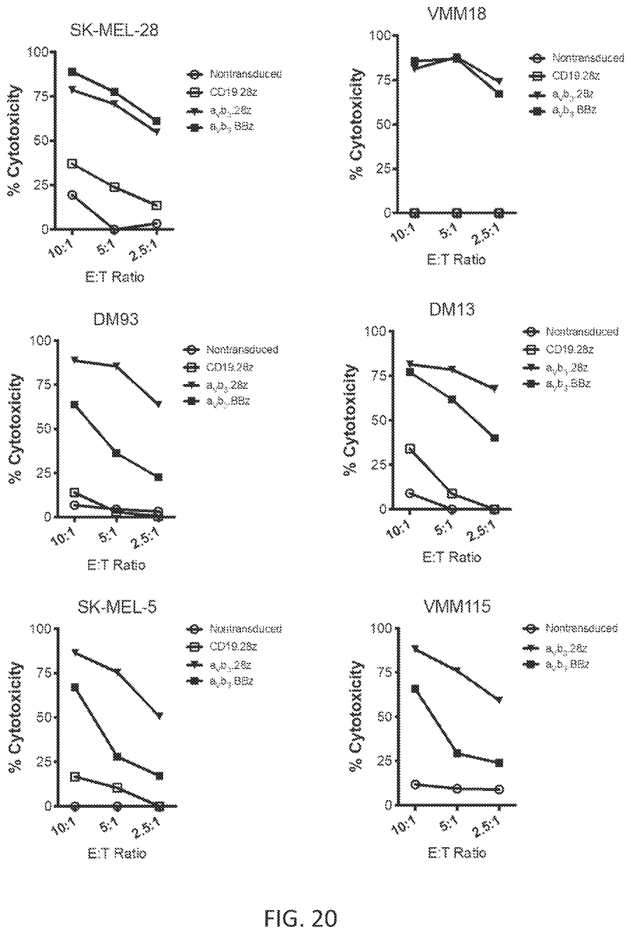

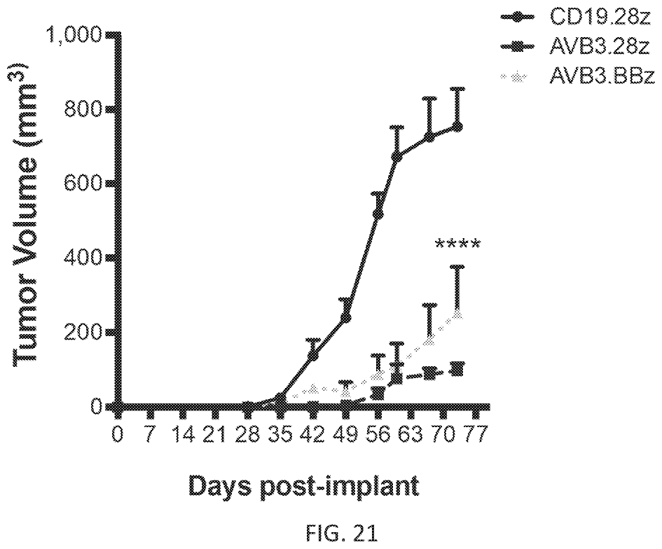

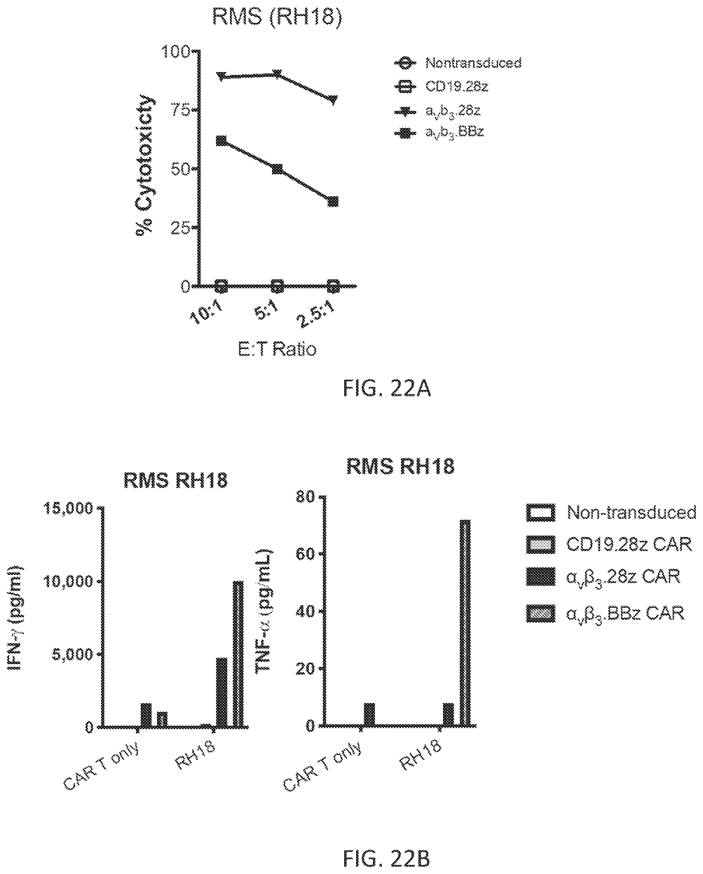

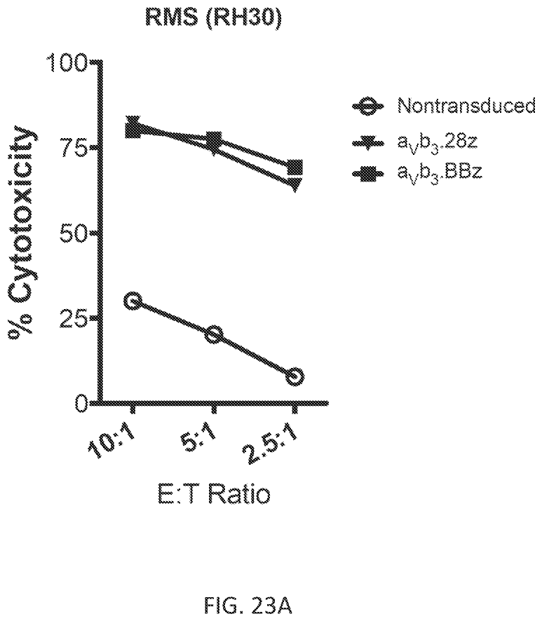

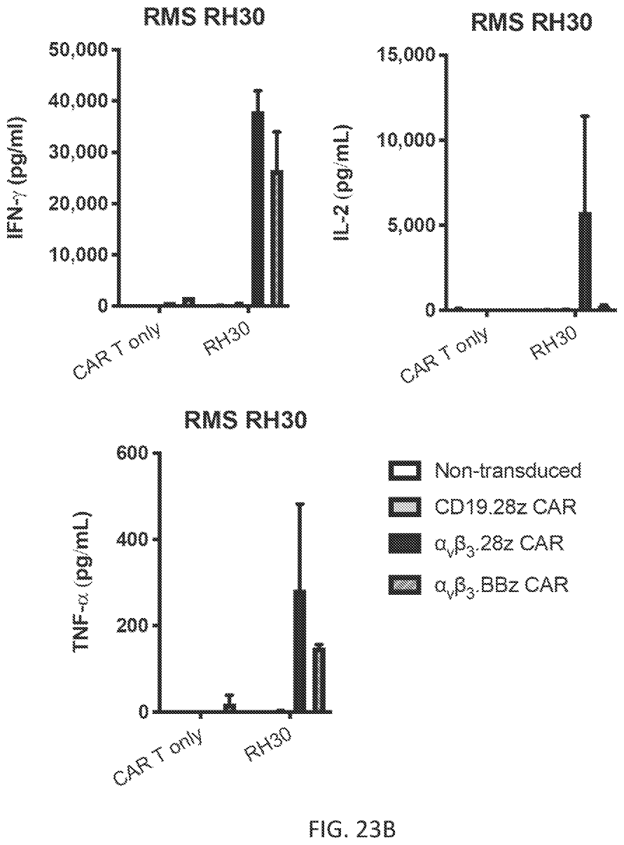

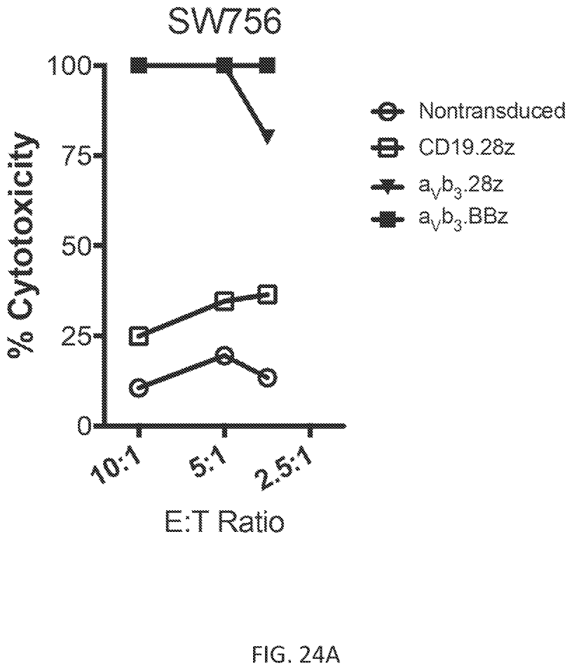

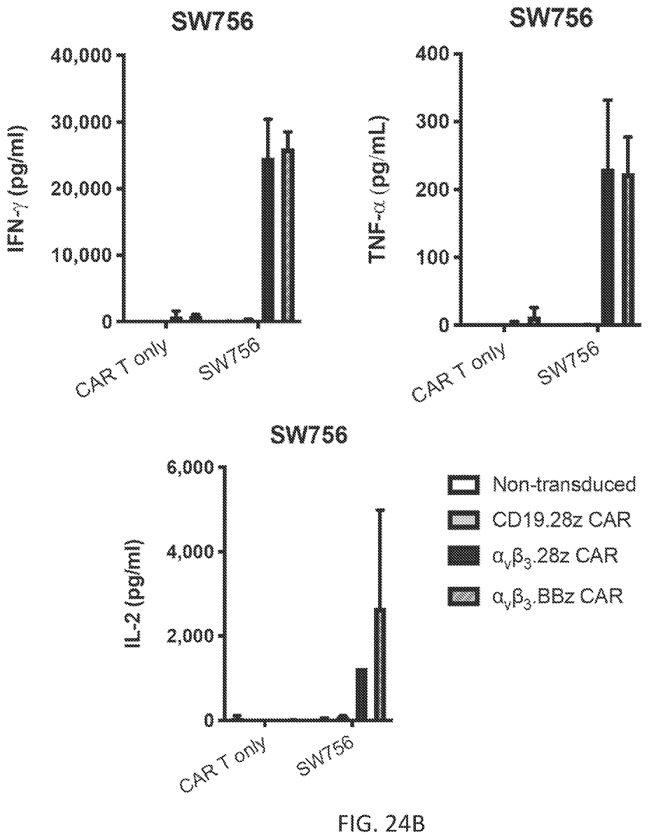

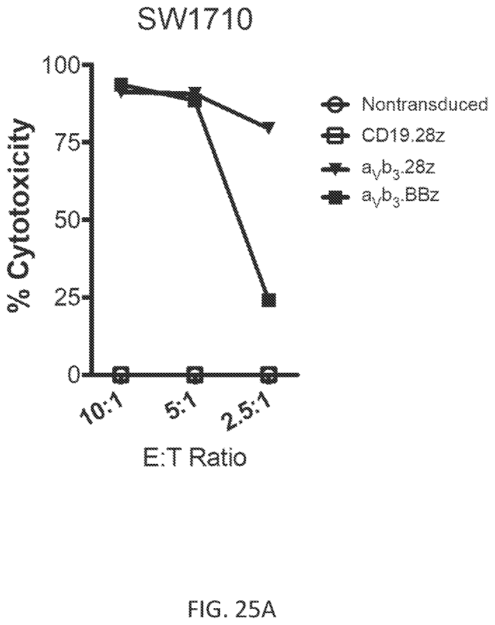

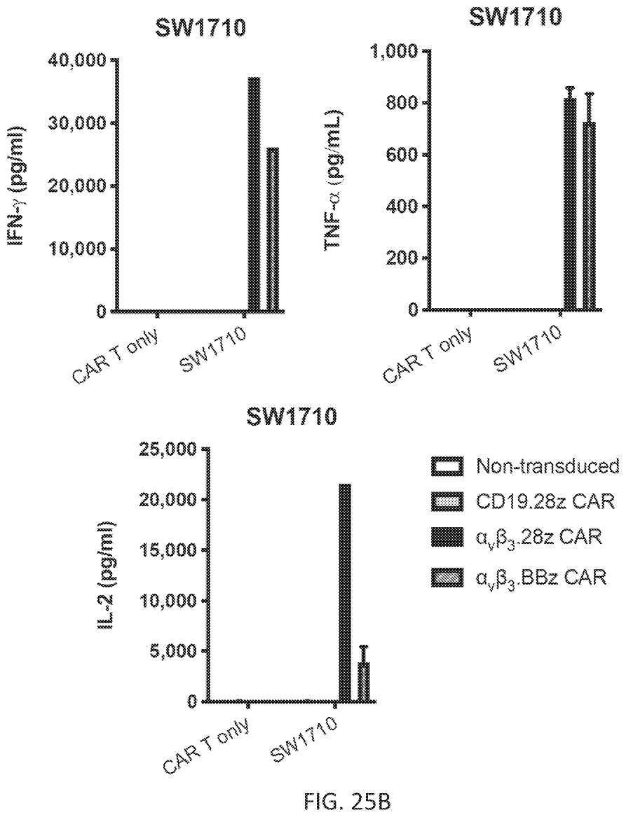

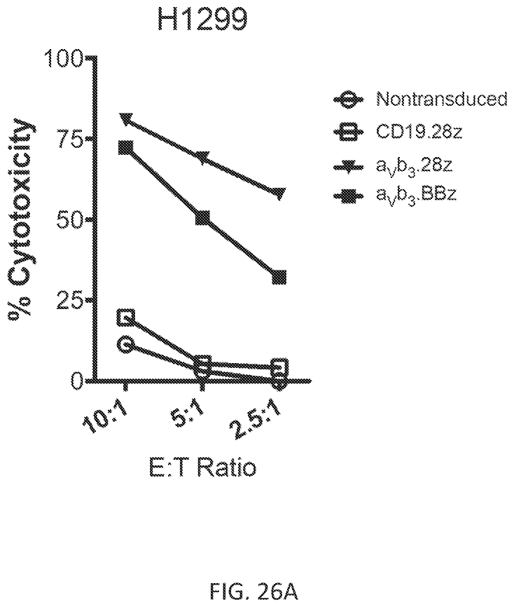

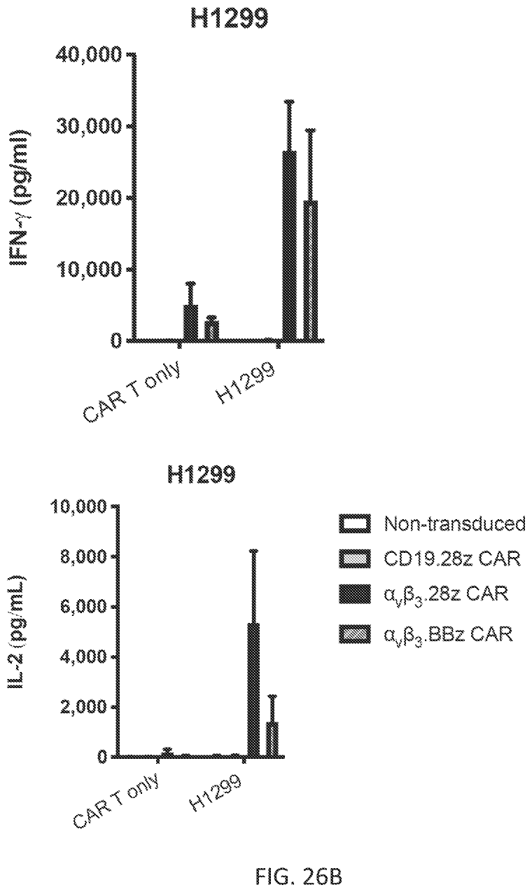

Potent in vitro anti-tumor activity for the αᵥβ₃.28z CAR T cells and αᵥβ₃.BBz CAR T cells was also observed against triple-negative breast cancer tumor cells, melanoma cells, rhabdomyosarcoma (RMS) tumor cells, cervical squamous cell carcinoma tumor cells, bladder tumor cells, and non-small cell lung (NSCL) cancer cells. See FIGS. 17A, 17B, 20, 22A, 22B, 23A, 23B, 24A, 24B, 25A, 25B, 26A, and 26B. For the triple-negative breast, RMS, melanoma, cervical squamous cell carcinoma, NSCL, and bladder tumor cells, in vitro anti-tumor cytotoxicity was evaluated by measuring electrical impedance of adherent tumor cells. In addition, in vivo anti-tumor activity of the αᵥβ₃.28z CAR T cells and αᵥβ₃.BBz CAR T cells was also observed against triple-negative breast cancer tumor cells and melanoma cells. See FIGS. 18A, 18B, 18C, 19, and 21.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) Basic local alignment search tool. J. Mol. Biol., 215:403-410.
Altschul et al. (1990b) Protein database searches for multiple alignments. Proc. Natl. Acad. Sci. USA, 87(14):5509-5513.
Altschul et al. (1997) Gapped BLAST and PSI-BLAST: a new generation of protein database search programs. Nucleic Acids Res., 25:3389-3402.
Bowie et al. (1990) Science, 247:1306-1310.
Bird et al. (1988) Single-chain antigen-binding proteins. Science, 242:423-426.
Bruggermann et al. (1993) Year in Immunol., 7:33.
Carter et al. (1992) Proc. Natl. Acad. Sci. USA, 89:4285.
Chothia et al. (1987) J. Mol. Biol., 196:901.
Chou & Fasman (1974) Biochemistry, 13:222-245.
Chou & Fasman (1978) Ann. Rev. Biochem., 47: 251-276.
Chou & Fasman (1979) Biophys. J., 26:367-384.
Cougot, et al. (2001) Trends in Biochem. Sci., 29:436-444.
Dayhoff et al. (in Atlas of Protein Sequence and Structure 1978, National Biomedical Research Foundation, Washington D.C., United States of America.
Devereux et al. (1984) A comprehensive set of sequence analysis programs for the VAX. Nucl. Acids Res., 12:387-395.
Duchosal et al. (1992) Nature, 355:258.
Elango, et al. (2005) Biochim. Biophys. Res. Commun., 330:958-966.
European Patent Nos. EP 239,400; EP 519,596; EP 592,106.
Fu et al. (2013) Genetically modified T cells targeting neovasculature efficiently destroy tumor blood vessels, shrink established solid-tumors and increase nanoparticle delivery. Int. J. Cancer, 133:2483-2492.
Ghosh et al. (1991) Glycobiology, 5: 505-10.
Griffith et al. (1993) EMBO J., 12:725-734.
Gross & Mienhofer (1981) The Peptides, Vol. 3, Academic Press, New York, N.Y., United States of America, pp. 3-88.
Harlow & Lane (1988) Antibodies, A Laboratory Manual, Cold Spring Harbor Laboratory Publications, Cold Spring Harbor, N.Y., United States of America.
Hashizume et al. (2012) Characterization of a diffuse intrinsic pontine glioma cell line: Implications for future investigations and treatment. J Neurooncol., 110(3):305-13.
Hoogenboom et al. (1991) J. Mol. Biol., 227:381.
Huston et al. (1988) Protein engineering of antibody binding sites: recovery of specific activity in an anti-digoxin single-chain Fv analogue produced in Escherichia coli. Proc. Natl. Acad. Sci. USA, 85:5879-5883.
Jakobovits et al. (1993) Proc. Natl. Acad. Sci. USA, 90:2551.
Jakobovits et al. (1993) Nature, 362:255-258.
Johnson, Kevin S, and Chiswell, David J. (1993) Current Opinion in Structural Biology, 3:564-571.
Johnson & Overington (1993) J. Mol. Biol., 233:716-738
Jones et al. (1986) Replacing the complementarity-determining regions in a human antibody with those from a mouse. Nature, 321:522-525.
Kallies et al. (2020) Precursor exhausted T cells: key to successful immunotherapy. Nat Rev Immunol., 20: 128-136.
Karlin & Altschul (1990) Methods for assessing the statistical significance of molecular sequence features by using general scoring schemes. Proc. Natl. Acad. Sci. USA, 87:2264-2268.
Karlin & Altschul (1993) Applications and statistics for multiple high-scoring segments in molecular sequences. Proc. Natl. Acad. Sci. USA, 90:5873-5877.
Kratchmarov et al. (2018) TCF1 expression marks self-renewing human CD8+ T cells. Blood Adv., 2(14):1685-90.
Kluiver et al. (2020) Invaders Exposed: Understanding and Targeting Tumor Cell Invasion in Diffuse Intrinsic Pontine Glioma. Front. Oncol., 10:1-13.
Kyte & Doolittle (1982) J. Mol. Biol., 157:105-132.
Lonberg and Huszar (1995) Int. Rev. Immunol., 13:65-93.
Marigil M et al. (2017) Development of a DIPG orthotopic model in mice using an implantable guide-screw system. PLoS One, 12(1):1-10.
Marks et al. (1991) J. Mol. Biol., 222:581-597.
McCafferty et al. (1990) Nature, 348:552-553.
Mount et al. (2018) Potent antitumor efficacy of anti-GD2 CAR T cells in H3-K27M+ diffuse midline gliomas. Nat. Med., 24:572-579.
Nacheva and Berzal-Herranz (2003) Eur. J. Biochem., 270:1485-65.
Nishikawa, et al. (2001) Hum Gene Ther., 12(8):861-70.
Padlan (1991) Molecular Immunology, 28(4/5):489-498.
Paul (1993) Fundamental Immunology, 3rd Ed., Raven Press, New York, N.Y., United States of America.
PCT International Patent Application Publication Nos. WO 1991/09967; WO 1991/10741; WO 1996/33735; WO 1996/34096; WO 1998/16654; WO 1998/24893; WO 1998/46645; WO 1998/50433; WO 2001/29058; WO 2001/96584; WO 2007/019107; WO 2007/030652; WO 2007/089798; WO 2008/060374.
Presta et al. (1993) Humanization of an antibody directed against IgE. J. Immunol., 1993 151:2623.
Qin et al. (2017) Neural Precursor-Derived Pleiotrophin Mediates Subventricular Zone Invasion by Glioma. Cell, 170(5):845-859.e19.
Riechmann et al. (1988) Reshaping human antibodies for therapy. Nature, 332:323-327.
Roder et al. (1986) Methods Enzymol., 121:140-167.
Roguska et al. (1994) Proc. Natl. Acad. Sci., USA, 91:969-973.
Rosenberg et al. (1988) New Eng. J. of Med., 319:1676.
Sambrook et al. (2001, Molecular Cloning: A Laboratory Manual, Cold Spring Harbor Laboratory, New York).
Schenborn and Mierendorf (1985) Nuc. Acids Res., 13:6223-36.
Siddiqui et al. (2019) Intratumoral Tcf1+PD-1+CD8+ T Cells with Stem-like Properties Promote Tumor Control in Response to Vaccination and Checkpoint Blockade Immunotherapy. Immunity, 50(1):195-211.e10.
Sims et al. (1993) A humanized CD18 antibody can block function without cell destruction. J. Immunol., 151:2296-2308.
Stepinski, et al. (2001) RNA, 7:1468-95.
Studnicka et al. (1994) Protein Engineering, 7(6):805-814.
Ui-Tei et al. (2000) FEBS Letters, 479: 79-82.
U.S. Patent Application Publication Nos. 2003/0017534; 2004/0101519; 2006/0034810; 2006/0121005; 2018/0298087, 2018/0312588, 2018/0346564, 2019/0151448.
U.S. Pat. Nos. 4,444,887; 4,554,101; 4,716,111; 4,816,567; 5,225,539; 5,229,275; 5,350,674; 5,399,346; 5,413,923; 5,482,856; 5,530,101; 5,545,806; 5,565,332; 5,567,610; 5,569,825; 5,573,905; 5,580,859; 5,585,089; 5,585,362; 5,589,466; 5,625,126; 5,633,425; 5,661,016; 5,814,318; 5,858,358; 5,883,223; 5,939,598 5,989,598; 6,326,193; 6,352,694; 6,479,284; 6,534,055; 6,677,436; 6,692,964; 6,797,514; 6,867,041; 6,887,466; 6,905,680; 6,905,681; 6,905,874; 7,060,808; 7,067,318; 7,144,575; 7,172,869; 7,175,8431 7,232,566; 7,906,625; 8,398,980; 8,436,150; 8,796,439; 10,253,111.
Vaughan et al. (1996) Nature Biotech., 14:309.
Venkatesh et al. (2017) Targeting neuronal activity-regulated neuroligin-3 dependency in high-grade glioma. Nature, 549(7673):533-7.
Wieland et al. (2017) TCF1+ hepatitis C virus-specific CD8+ T cells are maintained after cessation of chronic antigen stimulation. Nat. Commun., 8:1-13.
Winter & Milstein (1991) Man-made antibodies. Nature, 349:293-299.
Wu et al. (1999) J. Mol. Biol., 294:151.
Zhang et al. (2020) Cell Dysfunction and exhaustion in cancer. Front. Cell Dev., 8:17.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. An isolated nucleic acid encoding a chimeric antigen receptor (CAR), wherein the CAR comprises an integrin alphaᵥ beta₃ (αᵥβ₃) binding domain, a transmembrane domain, a cytoplasmic co-stimulatory (CS) domain, and an intracellular signaling domain, wherein the CAR is free of an extracellular immunoglobulin G4 (IgG4) hinge domain and/or wherein the integrin αᵥβ₃ binding domain is a single chain variable fragment (scFV) comprising a heavy chain variable region (V_{H}) encoded by SEQ ID NO: 3 or by a sequence at least 90% identical to SEQ ID NO: 3 and a light chain variable region (V_{L}) encoded by SEQ ID NO: 5 or by a sequence at least 90% identical to SEQ ID NO: 5.

2. The isolated nucleic acid of claim 1, wherein the CAR is free of an IgG4 hinge domain.

3. The isolated nucleic acid of claim 1 or claim 2, wherein the transmembrane domain comprises a cluster of differentiation 8 alpha (CD8α) transmembrane domain.

4. The isolated nucleic acid of any one of claims 1-3, wherein the transmembrane domain is a peptide encoded by SEQ ID NO: 7 or by a sequence at least 90% identical to SEQ ID NO: 7.

5. The isolated nucleic acid of any one of claims 1-4, wherein the integrin αᵥβ₃ binding domain comprises a scFV comprising a V_{H} encoded by SEQ ID NO: 3 and a V_{L} encoded by SEQ ID NO: 5.

6. The isolated nucleic acid of claim 5, wherein the integrin αᵥβ₃ binding domain comprises a linker peptide located between SEQ ID NO: 3 and SEQ ID NO: 5, optionally wherein said linker peptide is encoded by SEQ ID NO: 4 or by a sequence at least 90% identical to SEQ ID NO: 4.

7. The isolated nucleic acid of any one of claims 1-6, wherein the integrin αᵥβ₃ binding domain is attached to the transmembrane domain via a peptide encoded by SEQ ID NO: 6 or by a sequence at least 90% identical to SEQ ID NO: 6.

8. The isolated nucleic acid of any one of claims 1-7, wherein the cytoplasmic CS domain comprises one or both of a cluster of differentiation 28 (CD28) signaling domain and a tumor necrosis factor superfamily member 9 (4-1BB) signaling domain, optionally wherein the CD28 signaling domain is encoded by SEQ ID NO: 8 or a sequence at least 90% identical to SEQ ID NO: 8 and/or wherein the 4-1 BB signaling domain is encoded by SEQ ID NO: 9 or a sequence at least 90% identical to SEQ ID NO: 9.

9. The isolated nucleic acid of any one of claims 1-8, wherein the intracellular signaling domain is a cluster of differentiation 247 (CD3ζ) activation domain, optionally wherein the intracellular signaling domain is the peptide encoded by SEQ ID NO: 10 or a sequence at least 90% identical to SEQ ID NO: 10.

10. The isolated nucleic acid of any one of claims 1-9, further comprising a signal peptide, optionally wherein the signal peptide is a granulocyte-macrophage colony-stimulating factor (GM-CSF) signal peptide, further optionally wherein the signal peptide is human GM-CSF signal peptide encoded by SEQ ID NO: 2.

11. The isolated nucleic acid of any one of claims 1-9 comprising, in the following 5′ to 3′ order:
(i) SEQ ID NO: 2;
(ii) SEQ ID NO: 3;
(iii) SEQ ID NO: 4;
(iv) SEQ ID NO: 5;
(v) SEQ ID NO: 6;
(vi) SEQ ID NO: 7;
(vii) SEQ ID NO: 8 and/or SEQ ID NO: 9; and
(vii) SEQ ID NO: 10.

12. A nucleic acid construct comprising the isolated nucleic acid of any one of claims 1-11, operably linked to a promoter.

13. A vector comprising the isolated nucleic acid of any one of claims 1-11, optionally wherein the vector is a retroviral vector.

14. A chimeric antigen receptor (CAR), wherein the CAR comprises an integrin alpha betas (αᵥβ₃) binding domain, a transmembrane domain, at least one cytoplasmic co-stimulatory (CS) domain, and an intracellular signaling domain, wherein the CAR is free of an extracellular immunoglobulin G4 (IgG4) hinge domain and/or wherein the integrin αᵥβ₃ binding domain is a single chain variable fragment (scFV) comprising:
(i) a heavy chain variable region (V_{H}) comprising SEQ ID NO: 13 or an amino acid sequence at least 90% identical to SEQ ID NO: 13, and a light chain variable region (V_{L}) comprising SEQ ID NO: 15 or an amino acid sequence at least 90% identical to SEQ ID NO: 15; and/or
(ii) V_{H} comprising complementarity determining regions CDR-VH1, CDR-VH2, and CDR-VH3, wherein CDR-VH1, CDR-VH2, and CDR-VH3 comprise, consist essentially of, or consist of amino acids 31-35, 50-66, and 99-106 of SEQ ID NO: 13, respectively; and/or
(iii) V_{L} comprising complementarity determining regions CDR-VL1, CDR-VL2, and CDR-VL3, wherein CDR-VL1, CDR-VL2, and CDR-VL3 comprise, consist essentially of, or consist of amino acids 24-34, 50-56, and 88-97 of SEQ ID NO: 15, respectively.

15. The CAR of claim 14, wherein the CAR is free of an IgG4 hinge domain.

16. The CAR of claim 14 or claim 15, wherein the transmembrane domain comprises a cluster of differentiation 8 alpha (CD8α) transmembrane domain.

17. The CAR of any one of claims 14-16, wherein the transmembrane domain comprises SEQ ID NO: 7 or an amino acid sequence at least 90% identical to SEQ ID NO: 17.

18. The CAR of any one of claims 14-17, wherein the integrin αᵥβ₃ binding domain comprises a scFV comprising a V_{H} comprising SEQ ID NO: 13 and a V_{L} comprising SEQ ID NO: 15.

19. The CAR of claim 18, wherein the integrin αᵥβ₃ binding domain comprises a linker peptide located between SEQ ID NO: 13 and SEQ ID NO: 15, optionally wherein said linker peptide comprises or consists of SEQ ID NO: 14 or an amino acid sequence at least 90% identical to SEQ ID NO: 14.

20. The CAR of any one of claims 14-19, wherein the integrin αᵥβ₃ binding domain is attached to the transmembrane domain via a peptide comprising SEQ ID NO: 16 or by an amino acid sequence at least 90% identical to SEQ ID NO: 16.

21. The CAR of any one of claims 14-20, wherein the cytoplasmic CS domain comprises one or both of a cluster of differentiation 28 (CD28) signaling domain and a tumor necrosis factor superfamily member 9 (4-1BB) signaling domain, optionally wherein the CD28 signaling domain comprises SEQ ID NO: 18 or an amino acid sequence at least 90% identical to SEQ ID NO: 18 and/or wherein the 4-1 BB signaling domain comprises SEQ ID NO: 19 of an amino acid sequence at least 90% identical to SEQ ID NO: 19.

22. The CAR of any one of claims 14-21, wherein the intracellular signaling domain is a cluster of differentiation 247 (CD3ζ) activation domain, optionally wherein the intracellular signaling domain comprises SEQ ID NO: 20 or an amino acid sequence at least 90% identical to SEQ ID NO: 20.

23. The CAR of any one of claims 14-22, further comprising a signal peptide, optionally wherein the signal peptide is a granulocyte-macrophage colony-stimulating factor (GM-CSF) signal peptide, further optionally wherein the signal peptide comprises SEQ ID NO: 12.

24. The CAR of any one of claims 14-23 comprising, in the following N-terminus to C-terminus order:
(i) SEQ ID NO: 12;
(ii) SEQ ID NO: 13;
(iii) SEQ ID NO: 14;
(iv) SEQ ID NO: 15;
(v) SEQ ID NO: 16;
(vi) SEQ ID NO: 17;
(vii) SEQ ID NO: 18 and/or SEQ ID NO: 19; and
(vii) SEQ ID NO: 20.

25. An isolated polynucleotide encoding the CAR of any one of claims 14-24.

26. A genetically modified immune cell comprising the isolated nucleic acid of any one of claims 1-11 or 25 or a vector thereof.

27. The genetically modified immune cell of claim 26, expressing the CAR encoded by said isolated nucleic acid.

28. A genetically modified immune cell comprising the CAR of any one of claims 14-24.

29. A population of immune cells comprising a plurality of the genetically modified immune cells of any one of claims 26-28.

30. A pharmaceutical composition comprising the genetically modified immune cells according to any one of claims 26-28, and a pharmaceutically acceptable excipient.

31. A method of treating cancer in a subject in need thereof, comprising administering a therapeutically effective amount of the genetically modified immune cells according to any one of claims 26-28.

32. The method of claim 31, wherein the cancer is selected from a brain cancer, a breast cancer, a cervical cancer, a bladder cancer, a lung cancer, melanoma, neuroblastoma, and rhabdomyosarcoma (RMS), optionally wherein said lung cancer is a non-small cell lung cancer or wherein said breast cancer is a triple negative breast cancer.

33. The method of claim 32, wherein the cancer is a brain cancer.

34. The method of claim 33, wherein the brain cancer is a diffuse intrinsic pontine glioma (DIPG).

35. The method of claim 33, wherein the brain cancer is a glioblastoma (GBM).

36. The method of any one of claims 31-35, wherein the administering is performed intratumorally.

37. The method of any one of claims 31-35, wherein the administering is performed systemically.

38. A method of making an anti-integrin αvβ3 chimeric antigen receptor (CAR) immune cell, wherein the method comprises introducing into an immune cell a nucleic acid construct of claim 12 or a vector of claim 13.

39. The method of claim 38, wherein the nucleic acid construct or vector is introduced into the immune cell ex vivo.

40. The method of claim 39, wherein the vector is a virus vector, optionally wherein the virus vector is a retrovirus vector.

41. The method of any one of claims 38-40, wherein the immune cell is a T cell.

42. A method of treating cancer in a subject in need thereof, wherein the method comprises:
(a) obtaining a population of human immune cells;
(b) transducing at least a portion of the population of human immune cells with a vector of claim 13 to provide a population of transduced human immune cells; and
(c) administering the population of transduced human immune cells to the subject.

43. The method of claim 42, wherein the population of human immune cells is a population of human T cells obtained by:
(a1) obtaining cells from human blood serum, optionally wherein said cells comprise peripheral blood mononuclear cells (PBMCs); and
(a2) treating the cells from (a1) to isolate a population of cells enriched for central memory T cells; memory stem T cells, and naive T cells.

44. The method of claim 42 or claim 43, wherein the population of human immune cells are autologous to the subject.

45. The method of claim 42 or claim 43, wherein the population of human immune cells are allogenic to the subject.

46. The method of any one of claims 42-45, wherein the cancer is selected from a brain cancer, a breast cancer, a cervical cancer, a bladder cancer, a lung cancer, melanoma, neuroblastoma, and rhabdomyosarcoma (RMS), optionally wherein said lung cancer is a non-small cell lung cancer or wherein said breast cancer is a triple negative breast cancer.

47. The method of claim 46, wherein the cancer is a brain cancer.

48. The method of claim 47, wherein the brain cancer is a diffuse intrinsic pontine glioma (DIPG).

49. The method of claim 47, wherein the brain cancer is a glioblastoma (GBM).

50. The method of any one of claims 42-49, wherein the administering is performed intratumorally.

51. The method of any one of claims 42-49, wherein the administering is performed systemically.

52. A nucleic acid that encodes an antibody that binds to integrin alphaᵥ beta₃ (αᵥβ₃), wherein the nucleic acid comprises SEQ ID NO: 3 or a sequence at least 90% identical to SEQ ID NO: 3 and/or SEQ ID NO: 5 or a sequence at least 90% identical to SEQ ID NO: 5.

53. The nucleic acid of claim 52, wherein the antibody is a single-chain variable fragment (scFV).

54. The nucleic acid of claim 52 or claim 53, wherein the nucleic acid comprises the sequence R₁-R₂-R₃, wherein:
R₁ is SEQ ID NO: 3 or a sequence at least 90% identical thereto;
R₂ is SEQ ID NO: 4 or a sequence at least 90% identical thereto; and
R₃ is SEQ ID NO: 5 or a sequence at least 90% identical thereto.

55. An antibody that binds to integrin alphaᵥ beta₃ (αᵥβ₃), wherein the antibody comprises:
(i) a heavy chain variable region (V_{H}) comprising SEQ ID NO: 13 or a sequence at least 90% identical to SEQ ID NO: 13 and/or a light chain variable region (V_{L}) comprising SEQ ID NO: 15 or a sequence at least 90% identical to SEQ ID NO: 15; and/or
(ii) a V_{H} comprising complementarity determining regions CDR-VH1, CDR-VH2, and CDR-VH3, wherein CDR-VH1, CDR-VH2, and CDR-VH3 comprise, consist essentially of, or consist of amino acids 31-35, 50-66, and 99-106 of SEQ ID NO: 13, respectively; and/or
(iii) a V_{L} comprising complementarity determining regions CDR-VL1, CDR-VL2, and CDR-VL3, wherein CDR-VL1, CDR-VL2, and CDR-VL3 comprise, consist essentially of, or consist of amino acids 24-34, 50-56, and 88-97 of SEQ ID NO: 15, respectively.

56. The antibody of claim 55, wherein the antibody is a single-chain variable fragment (scFV).

57. The antibody of claim 55 or claim 56, wherein the antibody comprises a polypeptide sequence R₄-R₅-R₆, wherein:
R₄ is SEQ ID NO: 13 or a sequence at least 90% identical thereto;
R₅ is SEQ ID NO: 14 or a sequence at least 90% identical thereto; and
R₆ is SEQ ID NO: 15 or a sequence at least 90% identical thereto.


SEQUENCE LISTING (23 sequences)

SEQ ID NO: 1 - DNA, 15 bp, Artificial sequence
  synthetic sequence
tgaccacgtggcacc

SEQ ID NO: 2 - DNA, 66 bp, Homo sapiens
atgcttctcctggtgacaagccttctgctctgtgagttaccacacccagcattcctcctgatccca

SEQ ID NO: 3 - DNA, 351 bp, Artificial sequence
  Synthetic sequence
caggtgcagctggtggagtctgggggaggcgttgtgcagcctggaaggtccctgagactctcctgtgcagcctctggattcaccttcagtagctatgacatgtcttgggttcgccaggctccgggcaagggtctggagtgggtcgcaaaagttagtagtggtggtggtagcacctactatttagacactgtgcagggccgattcaccatctccagagacaatagtaagaacaccctatacctgcaaatgaactctctgagagccgaggacacagccgtgtattactgtgcaagacataactacggcagttttgcttactggggccaagggactacagtgactgtttctagt

SEQ ID NO: 4 - DNA, 60 bp, Artificial sequence
  synthetic sequence
ggaggcggcggaagcggaggcggcggaagcggaggcggcggaagcggaggcggcggaagc

SEQ ID NO: 5 - DNA, 321 bp, Artificial sequence
  synthetic sequence
gagattgtgctaactcagtctccagccaccctgtctctcagcccaggagaaagggcgactctttcctgccaggccagccaaagtattagcaaccacctacactggtatcaacaaaggcctggtcaagccccaaggcttctcatcaagtatcgttcccagtccatctctgggatccccgccaggttcagtggcagtggatcagggacagatttcaccctcactatctccagtctggagcctgaagattttgcagtctattactgtcaacagagtggcagctggcctcacacgttcggaggggggaccaaggtggaaattaag

SEQ ID NO: 6 - DNA, 15 bp, Artificial sequence
  synthetic sequence
gcatcaagcttgtcg

SEQ ID NO: 7 - DNA, 249 bp, Homo sapiens
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaac

SEQ ID NO: 8 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 9 - DNA, 141 bp, Homo sapiens
cgtttctctgttgttaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 10 - DNA, 339 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 11 - DNA, 15 bp, Artificial sequence
  synthetic sequence
tgacgttaacgcacc

SEQ ID NO: 12 - protein, 22 aa, Homo sapiens
MLLLVTSLLLCELPHPAFLLIP

SEQ ID NO: 13 - protein, 117 aa, Artificial sequence
  synthetic sequence
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHNYGSFAYWGQGTTVTVSS

SEQ ID NO: 14 - protein, 20 aa, Artificial sequence
  Synthetic sequence
GGGGSGGGGSGGGGSGGGGS

SEQ ID NO: 15 - protein, 107 aa, Artificial sequence
  synthetic sequence
EIVLTQSPATLSLSPGERATLSCQASQSISNHLHWYQQRPGQAPRLLIKYRSQSISGIPARFSGSGSGTDFTLTISSLEPEDFAVYYCQQSGSWPHTFGGGTKVEIK

SEQ ID NO: 16 - protein, 5 aa, Artificial sequence
  Synthetic sequence
ASSLS

SEQ ID NO: 17 - protein, 83 aa, Homo sapiens
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 18 - protein, 41 aa, Homo sapiens
RSKRSRLLHSDYMNMTPRRPGPTRKHYQPYAPPRDFAAYRS

SEQ ID NO: 19 - protein, 47 aa, Homo sapiens
RFSVVKRGRKKLLYIFKQPFMRPVQTTQEEDGCSCRFPEEEEGGCEL

SEQ ID NO: 20 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 5 aa, Artificial sequence
  Synthetic sequence
GGGGS

SEQ ID NO: 22 - protein, 107 aa, Mus musculus
ELVMTQTPATLSVTPGDSVSLSCRASQSISNHLHWYQQKSHESPRLLIKYASQSISGIPSRFSGSGSGTDFTLSINSVETEDFGMYFCQQSNSWPHTFGGGTKLEIK

SEQ ID NO: 23 - protein, 117 aa, Mus musculus
EVQLEESGGGLVKPGGSLKLSCAASGFAFSSYDMSWVRQIPEKRLEWVAKVSSGGGSTYYLDTVQGRFTISRDNAKNTLYLQMSSLNSEDTAMYYCARHNYGSFAYWGQGTLVTVSA